(19) United States
(12) Patent Application Publication
(10) Pub. No.: US 2015/0185184 A1
(43) Pub. Date: Jul. 2, 2015

(54) METHODS AND COMPOSITIONS FOR SEPARATING OR ENRICHING BLOOD CELLS

(71) Applicant: AVIVA BIOSCIENCES CORPORATION, San Diego, CA (US)

(72) Inventors: Antonio Guia, San Diego, CA (US); Douglas T. Yamanishi, San Diego, CA (US); Andrea Ghetti, San Diego, CA (US); Guoliang Tao, San Diego, CA (US); Huimin Tao, San Diego, CA (US); Ky Truong, San Diego, CA (US); Lei Wu, San Diego, CA (US); Xiaobo Wang, San Diego, CA (US)

(21) Appl. No.: 14/412,410

(22) PCT Filed: Jul. 5, 2013
(86) PCT No.: PCT/US2013/049476

§ 371 Date: Dec. 31, 2014

Related U.S. Application Data
(63) Continuation of application No. 13/844,085, filed on Mar. 15, 2013.
(60) Provisional application No. 61/668,990, filed on Jul. 6, 2012.

(51) Int. Cl. G01N 27/447; C23C 16/50; G01N 33/49; C23C 16/06; C23C 8/06; B05D 1/00; C23C 14/34; C23C 16/08

(52) U.S. Cl. CPC G01N 27/44791; C23C 14/3414; C23C 16/50; C23C 16/08; C23C 16/06; C23C 8/06; B05D 1/62; G01N 33/491

(57) ABSTRACT

The present invention provides a filtration chamber comprising a microfabricated filter enclosed in a housing, wherein the surface of said filter and/or the inner surface of said housing are modified by vapor deposition, sublimation, vapor-phase surface reaction, or particle sputtering to produce a uniform coating; and a method for separating cells of a fluid sample, comprising: a) dispensing a fluid sample into the filtration chamber disclosed herein; and b) providing fluid flow of the fluid sample through the filtration chamber, wherein components of the fluid sample flow through or are retained by the filter based on the size, shape, or deformability of the components.

### CROSS REFERENCE TO RELATED APPLICATIONS

This application claims benefit of priority to U.S. patent application Ser. No. 13/844,085 entitled “Methods and Compositions for Separating or Enriching Cells,” filed Mar. 15, 2013, now pending, which claims benefit of priority to U.S. Patent Application Ser. No. 61/668,990 entitled, “Methods and Compositions for Separating or Enriching Cells,” filed on Jul. 6, 2012. This application is related to U.S. patent application Ser. No. 11/777,962 entitled “Methods and Compositions for Detecting Rare Cells from a Biological Sample,” filed Jul. 13, 2007, now abandoned, U.S. patent application Ser. No. 60/831,156 entitled, “Methods and Compositions for Detecting Rare Cells from a Biological Sample,” filed Jul. 14, 2006, U.S. patent application Ser. No. 11/497,919 entitled “Methods and Compositions for Separating Rare Cells from Fluid Samples,” filed Aug. 2, 2006, now pending, U.S. patent application Ser. No. 60/704,601 entitled, “Improved Methods and Compositions for Separating Rare Cells From Fluid Samples,” filed Aug. 2, 2005, U.S. patent application Ser. No. 11/264,413 entitled “Methods and Compositions for Detecting Non-Hematopoietic Cells from a Blood Sample,” filed Oct. 31, 2005, now allowed, U.S. Provisional Patent application No. 60/704,601, filed Aug. 2, 2005, entitled “Improved Methods and Compositions for Separating Rare Cells from Fluid Samples,” U.S. patent application Ser. No. PCT/US2004/030359, entitled “Methods, compositions, and automated systems for separating rare cells from fluid samples,” filed Sep. 15, 2004, U.S. patent application Ser. No. 10/701,684, entitled “Methods, compositions, and automated systems for separating rare cells from fluid samples,” filed Nov. 4, 2003, now U.S. Pat. No. 7,166,443 B2, U.S. patent application Ser. No. 10/268,312, entitled “Methods, Compositions, and Automated Systems for Separating Rare Cells from Fluid Samples” filed Oct. 10, 2002, now U.S. Pat. No. 6,949,355 B2, U.S. Provisional Patent application No. 60/348,228, filed on Oct. 29, 2001, entitled “Methods and automated systems for separating rare cells from fluid samples”, U.S. Provisional Patent application No. 60/328,724, filed Oct. 11, 2001, entitled “Methods and automated systems for separating rare cells from fluid samples”, and U.S. Provisional Patent application No. 60/394,517, filed on Jul. 9, 2002, entitled “Methods and automated systems for separating rare cells from fluid samples.” The contents of the above referenced patent, applications and provisional applications are herein incorporated by reference in their entireties.

### TECHNICAL FIELD

The present invention relates generally to the field of bioseparation, and in particular to the field of biological sample processing.

### BACKGROUND OF THE INVENTION

Sample preparation is a necessary step for many genetic, biochemical, and biological analyses of biological and environmental samples. Sample preparation frequently requires the separation of sample components of interest from the remaining components of the sample. Such separations are often labor intensive and difficult to automate.

In many cases it is necessary to analyze relatively rare components of a sample. In this case, it may be necessary both to increase the concentration of the rare components to be analyzed, and to remove undesirable components of the sample that can interfere with the analysis of the components of interest. Thus, a sample must be “debulked” to reduce its volume, and in addition subjected to separation techniques that can enrich the components of interest. This is particularly true of biological samples, such as ascites fluid, lymph fluid, or blood, that can be harvested in large amounts, but that can contain minute percentages of target cells (such as virus-infected cells, anti-tumor T-cells, inflammatory cells, cancer cells, or fetal cells) whose separation is of critical importance for understanding the basis of disease states as well as for diagnosis and development of therapies.

Filtration has been used as a method of reducing the volume of samples and separating sample components based on their ability to flow through or be retained by the filter. Typically membrane filters are used in such applications in which the membrane filters have interconnected, fiber-like, structure distribution and the pores in the membrane are not discretely isolated; instead the pores are of irregular shapes and are connected to each other within the membrane. The so-called “pore” size really depends on the random tortuosity of the fluid-flow spaces (e.g., pores) in the membrane. While the membrane filters can be used for a number of separation applications, the variation in the pore size and the irregular shapes of the pores prevent them being used for precise filtration based on particle size and other properties.

Microfabricated filters have been made for certain cellular or molecular separation applications. These microfabricated structures do not have pores, but rather include channels that are microetched into one or more chips, by using “bricks” (see, for example, U.S. Pat. No. 5,837,115 issued Nov. 17, 1998 to Austin et al., incorporated by reference) or dams see, for example, U.S. Pat. No. 5,726,026 issued Mar. 10, 1998 to Wilding et al., incorporated by reference) that are built onto the surface of a chip. While these microfabricated filters have precise geometries, a limitation is that the filtration area of the filter is small, limited by the geometries of these filters, so that these filters can process only small volumes of the fluid sample.

Blood samples provide special challenges for sample preparation and analysis. Blood samples are easily obtained from subjects, and can provide a wealth of metabolic, diagnostic, prognostic, and genetic information. However, the great abundance of non-nucleated red blood cells, and their major component hemoglobin, can be an impediment to genetic, metabolic, and diagnostic tests. The debulking of red blood cells from peripheral blood has been accomplished using different layers of dense solutions (for example, see U.S. Pat. No. 5,437,987 issued Aug. 1, 1995 to Teng, Nelson N. H. et al). Long chain polymers such as dextran have been used to induce the aggregation of red blood cells resulting in the formation of long red blood cell chains (Sewchand L S, Canham P B. (1979) ‘Modes of Rouleaux formation of human red blood cells in polyvinylpyrrolidone and dextran solutions’ Can. J. Physiol. Pharmacol. 57(11):1213-22). However, the efficiency of these methods in removing red blood cells is less than optimal, especially where the separation or enrichment of rare cells, such as, for example, fetal cells from maternal blood or cancer cells from a patient, is desirable. Cell lysis techniques have also been used to remove red blood cells. However, the drawbacks of cell lysis techniques include nonspecific nucleated cell lysis, red blood cell debris as a result from cell lysis, and potential cell volume alteration (Resnitzky P, Reichman N. (1978) ‘Osmotic fragility of peripheral blood lymphocytes in chronic lymphatic leukemia and malignant lymphoma’ Blood 51(4):645-651).

Exfoliated cells in body fluids (e.g. sputum, urine, or even ascetic fluid or other effusions) present a significant opportunity for detection of precancerous lesions and for eradication of cancer at early stages of neoplastic development. For example, urine cytology is universally accepted as the noninvasive test for the diagnosis and surveillance of transitional cell carcinoma (Larsson et al (2001) Molecular Diagnosis 6: 181-188). However, in many cases, the cytologic identification of abnormal exfoliated cells has been limited by the number of abnormal cells isolated. For routine urine cytology (Ahrendt et al. (1999) J. Natl. Cancer Inst. 91: 299-301), the overall sensitivity is less than 50%, which varies with tumor grade, tumor stage, and urine collection and processing methods used. Molecular analysis (e.g. using in situ hybridization, PCR, microarrays, etc.) of abnormal exfoliated cells in body fluids based on molecular and genetic biomarkers can significantly improve the cytology sensitivity. Both biomarker studies and use of biomarkers for clinical practice would require a relatively pure exfoliated cell population enriched from body fluids comprising not only exfoliated cells but also normal cells, bacteria, body fluids, body proteins and other cell debris. Thus, there is an immediate need for developing an effective enrichment method for enriching and isolating exfoliated abnormal cells from body fluids.

Meye et al., Int. J. Oncol., 21(3):521-30 (2002) describes isolation and enrichment of urologic tumor cells in blood samples by a semi-automated CD45 depletion autoMACS protocol. Iinuma et al., Int. J. Cancer, 89(4):337-44 (2000) describes detection of tumor cells in blood using CD45 magnetic cell separation followed by nested mutant allele-specific amplification of p53 and K-ras genes in patients with colorectal cancer. In both studies, tumor cells were mixed with mononuclear cells (MNCs) isolated by Ficoll gradient centrifugation from a blood sample. Tumor cells were then enriched from MNCs by negative depletion using an anti-CD45 antibody.

Current approaches for enriching and preparing exfoliated cells from body fluids, e.g., blood samples, use media-based separation, antibody capture, centrifugation and membrane filtration. While these techniques are simple and straightforward, they suffer from a number of limitations, including: inadequate efficiency for rare cell enrichment; low sensitivity of rare cell detection; difficulty in handling large volume samples; inconsistency of the enrichment performance; and labor-intensiveness of separation procedure.

There is a need to provide methods and devices of sample preparation that are efficient and/or automatable that can process relatively large sample volumes, such as large volumes of biological fluid samples, and separate target cells. The present invention provides these and other benefits.

### BRIEF SUMMARY OF THE INVENTION

In some aspects, the present invention recognizes that diagnosis, prognosis, and treatment of many conditions can depend on the enrichment of target cells and/or cellular organelles from a complex fluid sample. Often, enrichment can be accomplished by one or more separation steps using a filtration device with slots that filter the cells according to the size, shape, deformability, binding affinity and/or binding specificity of the cells. For example, nucleated cells may be separated from non-nucleated red blood cells in peripheral blood samples using the filtration device. In comparison to removal of red blood cells based on cell lysis techniques, the filtration device disclosed in the present application may deplete red blood cells based on their size, shape, deformability, binding affinity and/or binding specificity, and minimize loss of nucleated cells due to nonspecific lysis. Further, it may achieve minimal alteration to nucleated cell volume and make a centrifugation step unnecessary.

In particular, the separation of fetal cells from maternal blood samples can greatly aid in the detection of fetal abnormalities or a variety of genetic conditions. In some aspects, the present invention recognizes that the enrichment or separation of rare malignant cells from patient samples, such as the isolation of cancerous cells from patient body fluid samples, can aid in the detection and typing of such malignant cells and therefore aid in diagnosis and prognosis, as well as in the development of therapeutic modalities for patients.

In a first aspect, the present invention provides a filtration chamber comprising a microfabricated filter enclosed in a housing, wherein the filtration chamber comprises an antechamber and a post-filtration subchamber, and the fluid flow path in the antechamber is substantially opposite to the fluid flow path in the post-filtration subchamber. In some embodiments, each of the antechamber and the post-filtration subchamber has an inflow port and/or an outflow port. In some embodiments, the antechamber comprises at least two inflow ports. In some embodiments, the antechamber comprises a suprafilter thereby creating a suprachamber, wherein the suprafilter may be placed on the side opposing the microfabricated filter. In some embodiments, the suprafilter, between the antechamber and the suprachamber, is sufficiently rigid to maintain its flatness under slow flow conditions. In some embodiments, the suprafilter comprises holes or slots with openings smaller than about 5 microns. In some embodiments, the inflow port and outflow port may be used interchangeably. In some embodiments, the microfabricated filter comprises one or more tapered slots. In some embodiments, the microfabricated filter comprises from about 100 to 5,000,000 tapered slots. In some embodiments, the thickness of the microfabricated filter is from about 20 to about 200 microns. In some embodiments, the thickness of the microfabricated filter is from about 40 to about 70 microns. In some embodiments, the tapered slots are from approximately 20 microns to 200 microns in length and from about 2 microns to about 16 microns in width, and the tapering of said slots is from about 0 degree to about 10 degrees, and the variation in slot size of said tapered slot is less than about 20%. In some embodiments, the size of the tapered slots varies by more than 20%. In some embodiments, the size of the tapered slots varies by more than 50%. In some embodiments, the size of the tapered slots varies by more than 100%. In some embodiments, the size of the tapered slots varies along the fluid flow path in the antechamber. In some embodiment, the post-filtration subchamber comprises at least two outflow ports. In some embodiments, the at least two outflow ports are arranged along the fluid flow path in the antechamber. In some embodiments, the filtration chamber comprises two or more electrodes. In some embodiments, the electrodes are placed on opposite sides of the microfabricated filter. In some embodiments, the electrodes are placed on the housing of the filtration chamber. In some embodiments, the electrodes are placed in the antechamber and/or the post-filtration subchamber. In some embodiments, the electrodes are incorporated or placed into one or more of the ports or connections that interact with the antechamber and/or the post-filtration subchamber. In some embodiments, the filtration chamber comprises at least one acoustic element. In some embodiments, the outflow port of the antechamber is connected to a collection chamber or collection well. In some embodiments, the housing comprises a top part and a bottom part, and the top part and the bottom part engage and may optionally bond together to form the filtration chamber. In some embodiments, the filtration chamber has a length of about 1 mm to about 10 cm, a width of about 1 mm to about 3 cm, and a depth of about 0.02 mm to about 20 mm. In some embodiments, the filtration chamber has a length of about 10 mm to about 50 mm, a width of about 5 mm to about 20 mm, and a depth of about 0.05 mm to about 2.5 mm. In some embodiments, the filtration chamber has a length of about 30 mm, a width of about 6 mm, and a depth of about 1 mm. In some embodiments, the housing containing it has a length of about 38 mm, a width of about 12 mm, and a depth of about 20 mm as outer dimensions. In some embodiments, the antechamber has a length of about 1 mm to about 10 cm, a width of about 1 mm to about 3 cm, and a depth of about 0.01 mm to about 10 mm. In some embodiments, the antechamber has a length of about 10 mm to about 50 mm, a width of about 5 mm to about 20 mm, and a depth of about 0.01 mm to about 1 mm. In some embodiments, the antechamber has a length of about 30 mm, a width of about 6 mm, and a depth of about 0.1-0.4 mm. In some embodiments, the volume of the antechamber is about 0.01 μL to about 5 mL. In some embodiments, the volume of the antechamber is about 1 μL to about 100 μL. In some embodiments, the volume of the antechamber is about 40-80 μL. In some embodiments, the post-filtration subchamber has a length of about 1 mm to about 10 cm, a width of about 1 mm to about 3 cm, and a depth of about 0.01 mm to about 1 cm. In some embodiments, the post-filtration subchamber has a length of about 10 mm to about 50 mm, a width of about 5 mm to about 20 mm, and a depth of about 0.2 mm to about 1.5 mm. In some embodiments, the post-filtration subchamber has a length of about 30 mm, a width of about 6.4 mm, and a depth of about 0.6-1 mm.

In a second aspect, the present invention provides a filtration chamber comprising a microfabricated filter enclosed in a housing, wherein the surface of said filter and/or the inner surface of said housing are modified by vapor deposition, sublimation, vapor-phase surface reaction, or particle sputtering to produce a uniform coating. In some embodiments, the filtration chamber comprises an antechamber and a post-filtration subchamber. In some embodiments, the antechamber comprises a suprafilter thereby creating a suprachamber. In some embodiments, the surface of the suprafilter is modified by vapor deposition, sublimation, vapor-phase surface reaction, or particle sputtering to produce a uniform coating. In some embodiments, the modification is by physical vapor deposition. In some embodiments, the modification is by plasma-enhanced chemical vapor deposition. In some embodiments, the vapor deposition is of a metal nitride or a metal halide. In some embodiments, the metal nitride is titanium nitride, silicon nitride, zinc nitride, indium nitride, and/or boron nitride. In some embodiments, the modification is by chemical vapor deposition. In some embodiments, the chemical vapor deposition is by Parylene or a derivative thereof. In some embodiments, the Parylene or derivative thereof is selected from the group consisting of Parylene, Parylene-N, Parylene-D, Parylene AF-4, Parylene SF, and Parylene HT. In some embodiments, the modification is by polytetrafluoroethylene (PTFE). In some embodiments, the modification is by amorphous Teflon or Teflon-AF. In some embodiments, the modification is by a perfluorocarbon. In some embodiments, the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl) silane and is in liquid form. In some embodiments, the filter and/or housing comprises silicon, silicon dioxide, glass, metal, carbon, ceramics, plastic, or a polymer. In some embodiments, the filter and/or housing comprises silicon nitride or boron nitride.

In a third aspect, the present invention provides a filtration chamber comprising a microfabricated filter enclosed in a housing, wherein the surface of said filter and/or the inner surface of said housing are modified by a metal nitride, a metal halide, a Parylene or derivative thereof, a polytetrafluoroethylene (PTFE), a Teflon-AF or a perfluorocarbon. In some embodiments, the filtration chamber comprises an antechamber and a post-filtration subchamber. In some embodiments, the antechamber comprises a suprafilter thereby creating a suprachamber. In some embodiments, the surface of the suprafilter is modified by a metal nitride, a metal halide, a Parylene or derivative thereof, a polytetrafluoroethylene (PTFE), a Teflon-AF or a perfluorocarbon. In some embodiments, the metal nitride is titanium nitride, silicon nitride, zinc nitride, indium nitride, and/or boron nitride. In some embodiments, the Parylene is selected from the group consisting of Parylene, Parylene-N, Parylene-D, Parylene AF-4, Parylene SF, and Parylene HT. In some embodiments, the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane, and the perfluorocarbon covalently binds the surface. In some embodiments, the filter and/or housing comprises silicon, silicon dioxide, glass, metal, carbon, ceramics, plastic, or a polymer. In some embodiments, the filter and/or housing comprises silicon nitride or boron nitride.

Also provided by the present invention is a filtration chamber comprising a microfabricated filter enclosed in a housing, wherein the filtration chamber comprises an antechamber and a post-filtration subchamber, and the fluid flow path in the antechamber is substantially opposite to the fluid flow path in the post-filtration subchamber, wherein the surface of the filter and/or the inner surface of said housing are modified by vapor deposition, sublimation, vapor-phase surface reaction, or particle sputtering to produce a uniform coating. Further provided in the present invention is a filtration chamber comprising a microfabricated filter enclosed in a housing, wherein the filtration chamber comprises an antechamber and a post-filtration subchamber, and the fluid flow path in the antechamber is substantially opposite to the fluid flow path in the post-filtration subchamber, wherein the surface of the filter and/or the inner surface of said housing are modified by a metal nitride, a metal halide, a Parylene, a polytetrafluoroethylene (PTFE), a Teflon-AF or a perfluorocarbon. In some embodiments, the filtration chamber provided herein may comprise at least two microfabricated filters. In some embodiments, the at least two microfabricated filters are arranged in tandem. Still further provided in the present invention is a filtration chamber comprising at least two filtration chambers disclosed herein arranged in tandem. In some embodiments, the antechambers of the at least two filtration chambers are in fluid connection. In some embodiments, the at least two filtration chambers share one microfabricated filter and/or suprafilter. In some embodiments, the slots of the filters within each filtration chamber are of different widths, and the filtration chambers are arranged in order of increasing slot widths.

In a fourth aspect, the present invention provides a cartridge comprising a filtration chamber disclosed herein. In some embodiments, the cartridge comprises at least two filtration chambers. In some embodiments, the cartridge comprises eight filtration chambers.

In a fifth aspect, the present invention provides an automated filtration unit for separating a target component in a fluid sample, comprising a filtration chamber disclosed herein. In some embodiments, the automated filtration unit comprises a control algorithm for controlling the fluid flow in the filtration chamber. In some embodiments, the automated filtration unit comprises at least two filtration chambers. In some embodiments, the at least two filtration chambers are arranged in tandem, and the filtration chambers comprise filters of increasing slot width. In some embodiments, the filters contain slot widths of increasing size along the fluidic path. In some embodiments, the filtration chamber comprises a suprachamber. In some embodiments, the post-filtration subchamber comprises multiple partitions each comprising an outflow port. In some embodiments, the outflow port from each partition of the post-filtration chamber is aligned with individual wells of a multi-well plate. In some embodiments, the wells are spaced about every 1-100 mm, e.g., about every 2.25 mm; about every 4.5 mm, or about every 9 or 18 mm. In some embodiments, the automated filtration unit comprises eight filtration chambers. In some embodiments, the automated filtration unit comprises a means for effecting fluid flow in the filtration chamber. In some embodiments, the means for effecting fluid flow is a fluidic pump. In some embodiments, the automated filtration unit comprises a means for collecting the separated target component.

In a sixth aspect, the present invention provides an automated system for separating and analyzing a target component in a fluid sample, comprising an automated filtration unit disclosed herein and an analysis apparatus connected to the filtration unit. In some embodiments, the analysis apparatus is a cell sorting device. In some embodiments, the analysis apparatus is a flow cytometer.

In a seventh aspect, the present invention provides a method for separating a target component in a fluid sample, comprising: a) dispensing a fluid sample into the filtration chamber disclosed herein; and b) providing a fluid flow of the fluid sample through the filtration chamber, wherein the target component of the fluid sample is retained by or passes through the filter. In some embodiments, the method comprises providing a fluid flow of the fluid sample through the antechamber of the filtration chamber and a fluid flow of a solution through the post-filtration subchamber of the filtration chamber, and optionally a fluid flow of a solution through the suprachamber of the filtration chamber. In some embodiments, the fluid sample is separated based on the size, shape, deformability, binding affinity and/or binding specificity of the components. In some embodiments, the fluid sample is dispensed through the inflow port of the antechamber. In some embodiments, the solution is introduced to the inflow port of the post-filtration subchamber. In some embodiments, the solution is introduced to the inflow port of the supra-filtration chamber. In some embodiments, the fluid sample is manipulated by a physical force effected via a structure that is external to the filter and/or a structure that is built-in on the filter. In some embodiments, the physical force is selected from the group consisting of a dielectrophoretic force, a traveling-wave dielectrophoretic force, a magnetic force, an acoustic force, an electrostatic force, a mechanical force, an optical radiation force and a thermal convection force. In some embodiments, the dielectrophoretic force or the traveling-wave dielectrophoretic force is effected via an electrical field produced by an electrode. In some embodiments, the acoustic force is effected via a standing-wave acoustic field or a traveling-wave acoustic field. In some embodiments, the acoustic force is effected via an acoustic field produced by piezoelectric material. In some embodiments, the acoustic force is effected via a voice coil or audio speaker. In some embodiments, the electrostatic force is effected via a direct current (DC) electric field. In some embodiments, the optical radiation force is effected via laser tweezers. In some embodiments, the sample is blood, an effusion, urine, a bone marrow sample, ascitic fluid, pelvic wash fluid, pleural fluid, spinal fluid, lymph, serum, mucus, sputum, saliva, semen, ocular fluid, extract of nasal, throat or genital swab, cell suspension from digested tissue, extract of fecal material, cultured cells of either mixed types and/or mixed sizes, or cells that contain contaminants or unbound reactants that need to be removed. In some embodiments, the fluid sample is a blood sample and the cells being separated are platelets and/or red blood cells (RBCs). In some embodiments, the fluid sample are cells that contain contaminants or unbound reactants that need to be removed, and the reactant is a labeling reagent for the cells. In some embodiments, the fluid sample is a blood sample and the cells being separated are non-hematopoietic cells, subpopulations of blood cells, fetal red blood cells, stem cells, or cancerous cells. In some embodiments, the fluid sample is an effusion or a urine sample and the cells being separated are cancerous cells or non-hematopoietic cells.

In an eighth aspect, the present invention provides a method of separating a target component in a fluid sample using the automated filtration unit disclosed herein, comprising: a) dispensing the fluid sample into the filtration chamber; and b) providing a fluid flow of the fluid sample through the filtration chamber, wherein the target component of the fluid sample is retained by or flows through the filter. In some embodiments, the fluid sample is separated based on the size, shape, deformability, binding affinity and/or binding specificity of the components. In some embodiments, the fluid sample in the antechamber flows substantially anti-parallel to the solution in the post-filtration subchamber. In some embodiments, the filter rate is about 0-1 mL/min. In some embodiments, the filter rate is about 10-500 μL/min. In some embodiments, the filter rate is about 80-140 μL/min. In some embodiments, the feed rate is about 1-10 times the filter rate.

In some embodiments, the method further comprises rinsing the retained components of the fluid sample with an additional sample-free rinsing reagent. In some embodiments, during the rinsing step the feed rate is less than or equal to the filter rate. In some embodiments, a rinsing reagent is introduced to the post-filtration subchamber. In some embodiments, the rinsing reagent is introduced to the antechamber and/or the suprachamber. In some embodiments, the method further comprises providing a labeling reagent to bind to the target component. In some embodiments, the labeling reagent is an antibody. In some embodiments, the labeling reagent is added to the collection chamber. In some embodiments, the labeling reagent is added to the antechamber and/or the suprachamber. In some embodiments, during the labeling step the fluid flow in the post-filtration subchamber is stopped. In some embodiments, the method further comprises removing the unbound labeling reagent. In some embodiments, the method further comprises recovering the target component in the collection chamber. In some embodiments, during recovering the feed rate is about 5-20 mL/min. In some embodiments, during the recovering step the outflow rate equals the inflow rate in the post-filtration subchamber. In some embodiments, during the recovering step the outflow is paused for about 50 ms. In some embodiments, the fluid sample is a blood sample, which comprises removing at least one type of undesirable component using a specific binding member. In some embodiments, the at least one undesirable component are white blood cells (WBCs). In some embodiments, the specific binding member selectively binds to WBCs and is coupled to a solid support. In some embodiments, the specific binding member is an antibody or an antibody fragment that selectively binds to WBCs. In some embodiments, the specific binding member is an antibody that selectively binds to CD3, CD11b, CD14, CD17, CD31, CD45, CD50, CD53, CD63, CD69, CD81, CD84, CD102 or CD166. In some embodiments, the specific binding member is an antibody that selectively binds to CD35 and/or CD50. In some embodiments, the method further comprises contacting the blood sample with a secondary specific binding member. In some embodiments, the secondary specific binding member is an antibody that selectively binds to CD31, CD36, CD41, CD42 (a, b or c), CD51, or CD51/61.

In a ninth aspect, the present invention provides a method of enriching and analyzing a component in a fluid sample using the automated system disclosed herein, comprising: a) dispensing the fluid sample into the filtration chamber; b) providing a fluid flow of the fluid sample through the antechamber of the filtration chamber and a fluid flow of a solution through the post-filtration subchamber of the filtration chamber, wherein the target component of the fluid sample is retained in the antechamber and non-target components flow through the filter into the post-filtration subchamber; c) labeling the target component; and d) analyzing the labeled target component using the analysis apparatus. In some embodiments, the method comprises providing fluid flow into the suprachamber. In some embodiments, the target component is a cell or cellular organelle. In some embodiments, the cell is a nucleated cell. In some embodiments, the cell is a rare cell.

### BRIEF DESCRIPTION OF THE FIGURES

FIG. 1 is the top view of a region of a microfabricated chip of an exemplary embodiment of the present invention. The dark areas are the precision manufactured slots in the filter that has a filtration area of 1 cm².

FIG. 2 is a schematic representation of a microfabricated filter of an exemplary embodiment of the present invention. A) the top view, showing an 18×18 mm² microfabricated filter having a filtration area (1) of 10×10 mm². B) an enlargement of a section of the top view, showing the slots (2) having dimensions of 4 microns×50 microns, with the center to center distance between slots of 12 microns, and their parallel alignment. C) a cross-sectional view of the microfabricated filter, with the slots extending through the filter substrate.

FIG. 3 depicts filters of an exemplary embodiment of the present invention having electrodes incorporated into their surfaces. A) a 20-fold magnification of a portion of a microfabricated filter having 2 micron slot widths. B) a 20-fold magnification of a portion of a microfabricated filter having 3 micron slot widths.

FIG. 4 depicts a cross section of a pore in a microfabricated filter of an exemplary embodiment of the present invention. The pore depth corresponds to the filter thickness. Y represents the right angle between the surface of the filter and the side of a pore cut perpendicularly through the filter, while X is the tapering angle by which a tapered pore differs in its direction or orientation through the filter from a non-tapered pore.

FIG. 5 depicts a filtration unit of an exemplary embodiment of the present invention having a microfabricated filter (3) separating the filtration chamber into an upper antechamber (4) and a post-filtration subchamber (5). The unit has valves to control fluid flow into and out of the unit: valve A (6) controls the flow of sample from the loading reservoir (10) into the filtration unit, valve B (7) controls fluid flow through the chamber by connection to a syringe pump, and valve C (8) is used for the introduction of wash solution into the chamber.

FIG. 6 is a diagram of an automated system of an exemplary embodiment of the present invention that comprises an inlet for the addition of a blood sample (11); a filtration chamber (12) that comprises acoustic mixing chips (13) and microfabricated filters (103); a magnetic capture column (14) having adjacent magnets (15); a mixing/filtration chamber (112); a magnetic separation chamber (16) comprising an electromagnetic chip (17), and a vessel for rare cell collection (18).

FIG. 7 depicts a three-dimensional perspective view of a filtration chamber of an exemplary embodiment of the present invention that has two filters (203) that comprise slots (202) and a chip having acoustic elements (200) (the acoustic elements may not be visible on the chip surface, but are shown here for illustrative purposes). In this simplified depiction, the width of the slots is not shown.

FIG. 8 depicts a cross-sectional view of a filtration chamber of an exemplary embodiment of the present invention having two filters (303) after filtering has been completed, and after the addition of magnetic beads (19) to a sample comprising target cells (20). The acoustic elements are turned on during a mixing operation.

FIG. 9 depicts a cross-sectional view of a feature of an automated system of an exemplary embodiment of the present invention: a magnetic capture column (114). Magnets (115) are positioned adjacent to the separation column.

FIG. 10 depicts a three-dimensional perspective view of a chamber (416) of an automated system of an exemplary embodiment of the present invention that comprises a multiple force chip that can separate rare cells from a fluid sample. The chamber has an inlet (429) and an outlet (430) for fluid flow through the chamber. A cut-away view shows the chip has an electrode layer (427) that comprises an electrode array for dielectrophoretic separation and an electromagnetic layer (417) that comprises electromagnetic units (421) an electrode array on another layer. Target cells (420) are bound to magnetic beads (419) for electromagnetic capture.

FIG. 11 shows a graph illustrating the theoretical comparison between the DEP spectra for an nRBC (Xs) and a RBC (circles) when the cells are suspended in a medium of electrical conductivity of 0.2 S/m.

FIG. 12 shows FISH analysis of nucleated fetal cells isolated using the methods of an exemplary embodiment of the present invention using a Y chromosome marker that has detected a male fetal cell in a maternal blood sample.

FIG. 13 shows a process flow chart for enriching fetal nucleated RBCs from maternal blood.

FIG. 14 is a schematic depiction of a filtration unit of an exemplary embodiment of the present invention.

FIG. 15 shows a model of an automated system of an exemplary embodiment of the present invention.

FIG. 16 depicts the filtration process of an automated system of an exemplary embodiment of the present invention. A) shows the filtration unit having a loading reservoir (510) connected through a valve (506) to a filtration chamber that comprises an antechamber (504) separated from a post-filtration subchamber (505) by a microfabricated filter (503). A wash pump (526) is connected to the lower chamber through a valve (508) for pumping wash buffer (524) through the lower subchamber. Another valve (507) leads to another negative pressure pump used to promote fluid flow through the filtration chamber and out through an exit conduit (530). A collection vessel (518) can reversibly engage the upper chamber (504). B) shows a blood sample (525) loaded into the loading reservoir (510). In C) the valve (507) that leads to a negative pressure pump used to promote fluid flow through the filtration chamber is open, and D) and E) show the blood sample being filtered through the chamber. In F) wash buffer introduced through the loading reservoir is filtered through the chamber. In G), valve (508) is open, while the loading reservoir valve (506) is closed, and wash buffer is pumped from the wash pump (526) into the lower chamber. In H) the filtration valve (507) and wash pump valve (508) are closed and in I) and J) the chamber is rotated 90 degrees. K) shows the collection vessel (518) engaging the antechamber (504) so that fluid flow generated by the wash pump (526) causes rare target cells (520) retained in the antechamber to flow into the collection tube.

FIG. 17 depicts a fluorescently labeled breast cancer cell in a background of unlabeled blood cells after enrichment by microfiltration. A) phase contrast microscopy of filtered blood sample. B) fluorescence microscopy of the same field shown in A.

FIG. 18 depicts two configurations of dielectrophoresis chips of an exemplary embodiment of the present invention. A) chip with interdigitated electrode geometry; B) chip with castellated electrode geometry.

FIG. 19 depicts a separation chamber of an exemplary embodiment of the present invention comprising a dielectrophoresis chip. A) Cross-sectional view of the chamber, B) top view showing the chip.

FIG. 20 is a graph illustrating the theoretical comparison between the DEP spectra for MDA231 cancer cells (solid line) T-lymphocytes (dashed line) and erythrocytes (small dashes) when the cells are suspended in a medium of electrical conductivity of 10 mS/m.

FIGS. 21 A and B depict breast cancer cells from a spiked blood sample retained on electrodes of an exemplary dielectrophoresis chip.

FIG. 22 depicts white blood cells of a blood sample retained on electrodes of an exemplary dielectrophoresis chip.

FIG. 23 is a schematic representation of a filtration unit of an automated system of an exemplary embodiment of the present invention. The filtration unit has a loading reservoir (610) connected through valve A (606) to a filtration chamber that comprises an antechamber (604) separated from a post-filtration subchamber (605) by a microfabricated filter (603). A suction-type pump can be attached through tubing that connects to the waste port (634), where filtered sample exits the chamber. A side port (632) can be used for attaching a syringe pump for pumping wash buffer through the lower subchamber (605). After the filtration process, the filtration chamber (including the antechamber (604), post-filtration subchamber (605), filter (603), and side port (632), all depicted within the circle in the figure) can rotate within the frame (636) of the filtration unit, so that enriched cells of the antechamber can be collected via the collection port (635).

FIG. 24 is a diagram showing the overall process of fetal cell enrichment from a blood sample, and the presence of enriched fetal cells in the supernatant of a second wash of the blood sample (box labeled Supernatant (W2)) and in the retained cells after the filtration step (box labeled Enriched cells). The diagram shows, from upper left to lower right, blood cell processing steps” two washes (W1 and W2), Selective sedimentation of red blood cells and removal of white blood cells with a combined reagent (AVIPrep+AVIBeads+Antibodies), Filtration of the supernatant of the sedimentation, and collection of enriched fetal cells. The diagram shows the level of enrichment of nucleated cells of various sample fractions during the procedure, and the sample fractions that were analyzed using FISH.

FIG. 25 shows a picture of the filter cartridge evaluated (right) and comparison to a regular disc syringe filter (left) with inserted top view image of the microfabricated silicon filter chip where the dark slots are the filter “pores” (a), described in U.S. Pat. No. 6,949,355; and a sketch of the filter cartridge structure (b).

FIG. 26 shows dot plots of the leucocytes isolated from whole blood with Lyse No Wash, Lyse Wash and filtration procedures (from top row to bottom row). P1 is the Trucount™ counting beads population and P2 is the leucocytes population gated on CD45+ cells.

FIG. 27 shows dot plots of blood stained with Multitest™ reagent processed by Lyse No Wash (LNW), Lyse Wash (LW), and filtration procedures (a); comparison of cell recovery of total leukocytes, major leukocyte populations, and major subpopulations of lymphocytes with LNW, LW, and filtration process (b). Recovery of CD45+ cells, lymphocyte, granulocyte, and monocyte was referenced to cell count obtained from ABX hematology analyzer (n=30) and recovery of T, NK, and B cells was compared to results from LNW sample (n=15).

FIG. 28 shows dot plots of whole blood stained with reagents in Viability kit, left panel is the result of whole blood lysed with ammonium chloride and right panel is the result of cells recovered from filtration (a); and dot plots of cells recovered from filtration stained with reagent in FITC Annexin V Apoptosis Detection Kit, left panel is the result of blood filtered within an hour after drawn and right panel is the result of blood filtered 8 h later after drawn (b).

FIG. 29 shows an exemplary embodiment of a cartridge.

FIG. 30 a-d show cell viability after ammonium chloride lysing.

FIG. 31 shows cell viability after filtration.

FIG. 32 illustrates an exemplary filter work process. In the exemplary embodiment of the process, there are two syringe pumps, one on the right, and the other on the bottom. Suction on the bottom one is simultaneous as output on the right one, but faster so that blood is drawn through the filter in the differential. Once filtering is done, the suction on the bottom one is turned off, and the nucleated cells are pushed back from the filter, which has been flipped upside down at this time to dispense the cells directly into a cytometry tube (as in step 6 but with the syringe replaced with a receiving cytometry tube).

FIG. 33 shows an exemplary embodiment of a filtration chamber wherein the antechamber and the post-filtration subchamber both have an inlet and an outlet that allow fluid to flow trough. In the exemplary embodiment depicted, the fluid in the antechamber flows antiparallel to the fluid in the post-filtration subchamber.

FIG. 34 shows an exemplary embodiment of a multiplex configuration of eight filtration chambers that each contains an independent filtration chamber with fluidic paths similar to that illustrated in FIG. 33.

FIG. 35 shows an exemplary embodiment of an automated system for separating and analyzing a target component of a fluid sample, wherein the sample may be collected by a syphon that is placed into the sample, and the sample may pass continuously through the antechamber and then be fed directly into an analytical instrument, which in this schematic is shown as the flow-cell of a flow cytometer.

FIG. 36 shows a schematic representation of an exemplary embodiment of a high-rinse capacity filtration chamber, wherein the same fluidic path present in FIG. 33 now has a rinsing reagent (buffer or buffer plus biomarker, or any suitable substance) introduced from above and passed through both filters to maximize the interaction between the sample and the bottom microfabricated filter.

FIG. 37 shows an exemplary embodiment of two filtration chambers in tandem, wherein the sample may be cleared of debris and small components in the first filtration chamber, then the second filtration chamber separates larger cells from smaller cells among those remaining. For example, leukocytes may be preferentially directed to the Recovery 1 port, and the larger tumor cells may continue to the Recovery 2 port.

FIG. 38 shows an exemplary embodiment of a filtration chamber with multiple recovery ports, wherein the microfabricated filter contains an array of slots with increasing width such that each port will output cells of progressively larger size and the ports may be spaced as to deliver their output directly into a multi-well screening plate.

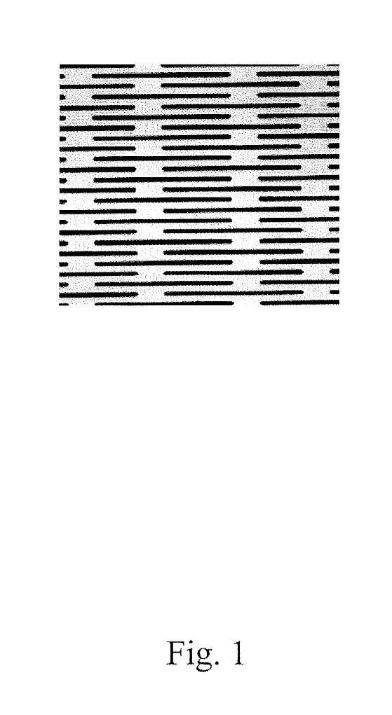

### DETAILED DESCRIPTION OF THE INVENTION

### Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a”, “an”, and “the” include plural references unless indicated otherwise. For example, “a” dimer includes one or more dimers.

A “component” of a sample or “sample component” is any constituent of a sample, and can be an ion, molecule, compound, molecular complex, organelle, virus, cell, aggregate, or particle of any type, including colloids, aggregates, particulates, crystals, minerals, etc. A component of a sample can be soluble or insoluble in the sample media or a provided sample buffer or sample solution. A component of a sample can be in gaseous, liquid, or solid form. A component of a sample may be a moiety or may not be a moiety.

A “moiety” or “moiety of interest” is any entity whose isolation, purification and/or manipulation is desirable. A moiety can be a solid, including a suspended solid, or can be in soluble form. A moiety can be a molecule. Molecules that can be manipulated include, but are not limited to, inorganic molecules, including ions and inorganic compounds, or can be organic molecules, including amino acids, peptides, proteins, glycoproteins, lipoproteins, glycolipoproteins, lipids, fats, sterols, sugars, carbohydrates, nucleic acid molecules, small organic molecules, or complex organic molecules. A moiety can also be a molecular complex, can be an organelle, can be one or more cells, including prokaryotic and eukaryotic cells, or can be one or more etiological agents, including viruses, parasites, or prions, or portions thereof. A moiety can also be a crystal, mineral, colloid, fragment, micelle, droplet, bubble, or the like, and can comprise one or more inorganic materials such as polymeric materials, metals, minerals, glass, ceramics, and the like. Moieties can also be aggregates of molecules, complexes, cells, organelles, viruses, etiological agents, crystals, colloids, or fragments. Cells can be any cells, including prokaryotic and eukaryotic cells. Eukaryotic cells can be of any type. Of particular interest are cells such as, but not limited to, white blood cells, malignant cells, stem cells, progenitor cells, fetal cells, and cells infected with an etiological agent, and bacterial cells. Moieties can also be artificial particles such polystyrene microbeads, microbeads of other polymer compositions, magnetic microbeads, and carbon microbeads.

As used herein, “manipulation” refers to moving or processing of the moieties, which results in one-, two- or three-dimensional movement of the moiety, whether within a single chamber or on a single chip, or between or among multiple chips and/or chambers. Moieties that are manipulated by the methods of the present invention can optionally be coupled to binding partners, such as microparticles. Non-limiting examples of the manipulations include transportation, capture, focusing, enrichment, concentration, aggregation, trapping, repulsion, levitation, separation, isolation or linear or other directed motion of the moieties. For effective manipulation of moieties coupled to binding partners, the binding partner and the physical force used in the method must be compatible. For example, binding partners with magnetic properties must be used with magnetic force. Similarly, binding partners with certain dielectric properties (e.g., plastic particles, polystyrene microbeads) must be used with dielectrophoretic force.

“Binding partner” refers to any substances that both bind to the moieties with desired affinity or specificity and are manipulatable with the desired physical force(s). Non-limiting examples of the binding partners include cells, cellular organelles, viruses, microparticles or an aggregate or complex thereof, or an aggregate or complex of molecules.

“Coupled” means bound. For example, a moiety can be coupled to a microparticle by specific or nonspecific binding. As disclosed herein, the binding can be covalent or noncovalent, reversible or irreversible.

As used herein, “the moiety to be manipulated is substantially coupled onto surface of the binding partner” means that a percentage of the moiety to be manipulated is coupled onto surface of the binding partner and can be manipulated by a suitable physical force via manipulation of the binding partner. Ordinarily, at least 0.1% of the moiety to be manipulated is coupled onto surface of the binding partner. Preferably, at least 1%, 5%, 10%, 20%, 30%, 40%, 50%, 60%, 70%, 80% or 90% of the moiety to be manipulated is coupled onto surface of the binding partner.

As used herein, “the moiety to be manipulated is completely coupled onto surface of the binding partner” means that at least 90% of the moiety to be manipulated is coupled onto surface of the binding partner. Preferably, at least 91%, 92%, 93%, 94%, 95%, 96%, 97%, 98%, 99% or 100% of the moiety to be manipulated is coupled onto surface of the binding partner.

A “specific binding member” is one of two different molecules having an area on the surface or in a cavity that specifically binds to and is thereby defined as complementary with a particular spatial and chemical organization of the other molecule. A specific binding member can be a member of an immunological pair such as antigen-antibody or antibody-antibody, can be biotin-avidin, biotin-streptavidin, or biotin-neutravidin, ligand-receptor, nucleic acid duplexes, IgG-protein A, DNA-DNA, DNA-RNA, RNA-RNA, and the like.

An “antibody” is an immunoglobulin molecule, and can be, as a non-limiting example, an IgG, an IgM, or other type of immunoglobulin molecule. As used herein, “antibody” also refers to a portion of an antibody molecule that retains the binding specificity of the antibody from which it is derived (for example, single chain antibodies or Fab fragments).

A “nucleic acid molecule” is a polynucleotide. A nucleic acid molecule can be DNA, RNA, or a combination of both. A nucleic acid molecule can also include sugars other than ribose and deoxyribose incorporated into the backbone, and thus can be other than DNA or RNA. A nucleic acid can comprise nucleobases that are naturally occurring or that do not occur in nature, such as xanthine, derivatives of nucleobases, such as 2-aminoadenine, and the like. A nucleic acid molecule of the present invention can have linkages other than phosphodiester linkages. A nucleic acid molecule of the present invention can be a peptide nucleic acid molecule, in which nucleobases are linked to a peptide backbone. A nucleic acid molecule can be of any length, and can be single-stranded, double-stranded, or triple-stranded, or any combination thereof.

“Homogeneous manipulation” refers to the manipulation of particles in a mixture using physical forces, wherein all particles of the mixture have the same response to the applied force.

“Selective manipulation” refers to the manipulation of particles using physical forces, in which different particles in a mixture have different responses to the applied force.

A “fluid sample” is any fluid from which components are to be separated or analyzed. A sample can be from any source, such as an organism, group of organisms from the same or different species, from the environment, such as from a body of water or from the soil, or from a food source or an industrial source. A sample can be an unprocessed or a processed sample. A sample can be a gas, a liquid, or a semi-solid, and can be a solution or a suspension. A sample can be an extract, for example a liquid extract of a soil or food sample, an extract of a throat or genital swab, or an extract of a fecal sample, or a wash of an internal area of the body.

A “blood sample” as used herein can refer to a processed or unprocessed blood sample, i.e., it can be a centrifuged, filtered, extracted, or otherwise treated blood sample, including a blood sample to which one or more reagents such as, but not limited to, anticoagulants or stabilizers have been added. An example of blood sample is a buffy coat that is obtained by processing human blood for enriching white blood cells. Another example of a blood sample is a blood sample that has been “washed” to remove serum components by centrifuging the sample to pellet cells, removing the serum supernatant, and resuspending the cells in a solution or buffer. Other blood samples include cord blood samples, bone marrow aspirates, internal blood or peripheral blood. A blood sample can be of any volume, and can be from any subject such as an animal or human. A preferred subject is a human.

A “rare cell” is a cell that is either 1) of a cell type that is less than 1% of the total nucleated cell population in a fluid sample, or 2) of a cell type that is present at less than one million cells per milliliter of fluid sample. A “rare cell of interest” is a cell whose enrichment is desirable.

A “white blood cell” or “WBC” is a leukocyte, or a cell of the hematopoietic lineage that is not a reticulocyte or platelet and that can be found in the blood of an animal or human. Leukocytes can include nature killer cells (“NK cells”) and lymphocytes, such as B lymphocytes (“B cells”) or T lymphocytes (“T cells”). Leukocytes can also include phagocytic cells, such as monocytes, macrophages, and granulocytes, including basophils, eosinophils and neutrophils. Leukocytes can also comprise mast cells.

A “red blood cell” or “RBC” is an erythrocyte. Unless designated a “nucleated red blood cell” (“nRBC”) or “fetal nucleated red blood cell” or nucleated fetal red blood cell, as used herein, “red blood cell” is used to mean a non-nucleated red blood cell.

“Neoplastic cells” or “tumor cells” refers to abnormal cells that have uncontrolled cellular proliferation and can continue to grow after the stimuli that induced the new growth has been withdrawn. Neoplastic cells tend to show partial or complete lack of structural organization and functional coordination with the normal tissue, and may be benign or malignant.

A “malignant cell” is a cell having the property of locally invasive and destructive growth and metastasis. Examples of “malignant cells” include, but are not limited to, leukemia cells, lymphoma cells, cancer cells of solid tumors, metastatic solid tumor cells (e.g., breast cancer cells, prostate cancer cells, lung cancer cells, colon cancer cells) in various body fluids including blood, bone marrow, ascitic fluids, stool, urine, bronchial washes etc.

A “cancerous cell” is a cell that exhibits deregulated growth and, in most cases, has lost at least one of its differentiated properties, such as, but not limited to, characteristic morphology, non-migratory behavior, cell-cell interaction and cell-signaling behavior, protein expression and secretion pattern, etc.

“Cancer” refers to a neoplastic disease that the natural course of which is fatal. Cancer cells, unlike benign tumor cells, exhibit the properties of invasion and metastasis and are highly anaplastic. Cancer cells include the two broad categories of carcinoma and sarcoma.

A “stem cell” is an undifferentiated cell that can give rise, through one or more cell division cycles, to at least one differentiated cell type.

A “progenitor cell” is a committed but undifferentiated cell that can give rise, through one or more cell division cycles, to at least one differentiated cell type. Typically, a stem cell gives rise to a progenitor cell through one or more cell divisions in response to a particular stimulus or set of stimuli, and a progenitor gives rise to one or more differentiated cell types in response to a particular stimulus or set of stimuli.

An “etiological agent” refers to any causal factor, such as bacteria, fungus, protozoan, virus, parasite or prion, that can infect a subject. An etiological agent can cause symptoms or a disease state in the subject it infects. A human etiological agent is an etiological agent that can infect a human subject. Such human etiological agents may be specific for humans, such as a specific human etiological agent, or may infect a variety of species, such as a promiscuous human etiological agent.

“Subject” refers to any organism, such as an animal or a human. An animal can include any animal, such as a feral animal, a companion animal such as a dog or cat, an agricultural animal such as a pig or a cow, or a pleasure animal such as a horse.

A “chamber” is a structure that is capable of containing a fluid sample, in which at least one processing step can be performed. In some embodiments, a chamber may have various dimensions and its volume may vary between 0.01 microliters and 0.5 liter.

A “filtration chamber” is a chamber through which or in which a fluid sample can be filtered.

A “filter” is a structure that comprises one or more pores or slots of particular dimensions (that can be within a particular range), that allow the passage of some sample components but not others from one side of the filter to the other, based on the size, shape, deformability, binding affinity and/or binding specificity of the components. A filter can be made of any suitable material that prevents passage of insoluble components, such as metal, ceramics, glass, silicon, plastics, polymers, fibers (such as paper or fabric), etc.

A “filtration unit” is a filtration chamber and the associated inlets, valves, and conduits that allow sample and solutions to be introduced into the filtration chamber and sample components to be removed from the filtration chamber. A filtration unit optionally also comprises a loading reservoir.

A “cartridge” is a structure that comprises at least one chamber that is part of a manual or automated system and one or more conduits for the transport of fluid into or out of at least one chamber. A cartridge may or may not comprise one or more chips.

An “automated system for separating a target component from a fluid sample” or an “automated system” is a device that comprises at least one filtration chamber, automated means for directing fluid flow through the filtration chamber, and at least one power source for providing fluid flow and, optionally, providing a signal source for the generation of forces on active chips. An automated system of the present invention can also optionally include one or more active chips, separation chambers, separation columns, or permanent magnets.

A “port” is an opening in the housing of a chamber through which a fluid sample can enter or exit the chamber. A port can be of any dimensions, but preferably is of a shape and size that allows a sample to be dispensed into a chamber by pumping a fluid through a conduit, or by means of a pipette, syringe, or other means of dispensing or transporting a sample.

An “inlet” is a point of entrance for sample, solutions, buffers, or reagents into a fluidic chamber. An inlet can be a port of a chamber, or can be an opening in a conduit that leads, directly or indirectly, to a chamber of an automated system.

An “outlet” is the opening at which sample, sample components, or reagents exit a fluidic chamber. The sample components and reagents that leave a chamber can be waste, i.e., sample components that are not to be used further, or can be sample components or reagents to be recovered, such as, for example, reusable reagents or target cells to be further analyzed or manipulated. An outlet can be a port of a chamber, but preferably is an opening in a conduit that, directly or indirectly, leads from a chamber of an automated system.

A “conduit” is a means for fluid to be transported from a container to a chamber of the present invention. Preferably a conduit directly or indirectly engages a port in the housing of a chamber. A conduit can comprise any material that permits the passage of a fluid through it. Conduits can comprise tubing, such as, for example, rubber, Teflon, or tygon tubing. Conduits can also be molded out of a polymer or plastic, or drilled, etched, or machined into a metal, glass or ceramic substrate. Conduits can thus be integral to structures such as, for example, a cartridge of the present invention. A conduit can be of any dimensions, but preferably ranges from 10 microns to 5 millimeters in internal diameter. A conduit is preferably enclosed (other than fluid entry and exit points), or can be open at its upper surface, as a canal-type conduit.

A “chip” is a solid substrate on which one or more processes such as physical, chemical, biochemical, biological or biophysical processes can be carried out, or a solid substrate that comprises or supports one or more applied force-generating elements for carrying out one or more physical, chemical, biochemical, biological, or biophysical processes. Such processes can be assays, including biochemical, cellular, and chemical assays; separations, including separations mediated by electrical, magnetic, physical, and chemical (including biochemical) forces or interactions; chemical reactions, enzymatic reactions, and binding interactions, including captures. The micro structures or micro-scale structures such as, channels and wells, bricks, dams, filters, electrode elements, electromagnetic elements, or acoustic elements, may be incorporated into or fabricated on the substrate for facilitating physical, biophysical, biological, biochemical, chemical reactions or processes on the chip. The chip may be thin in one dimension and may have various shapes in other dimensions, for example, a rectangle, a circle, an ellipse, or other irregular shapes. The size of the major surface of chips of the present invention can vary considerably, e.g., from about 1 mm² to about 0.25 m². Preferably, the size of the chips is from about 4 mm² to about 25 cm² with a characteristic dimension from about 1 mm to about 5 cm. The chip surfaces may be flat, or not flat. The chips with non-flat surfaces may include channels or wells fabricated on the surfaces. A chip can have one or more openings, such as pores or slots.

An “active chip” is a chip that comprises micro-scale structures that are built into or onto a chip that when energized by an external power source can generate at least one physical force that can perform a processing step or task or an analysis step or task, such as, but not limited to, mixing, translocation, focusing, separation, concentration, capture, isolation, or enrichment. An active chip uses applied physical forces to promote, enhance, or facilitate desired biochemical reactions or processing steps or tasks or analysis steps or tasks. On an active chip, “applied physical forces” are physical forces that, when energy is provided by a power source that is external to an active chip, are generated by micro-scale structures built into or onto a chip.

“Micro-scale structures” are structures integral to or attached on a chip, wafer, or chamber that have characteristic dimensions of scale for use in microfluidic applications ranging from about 0.1 micron to about 20 mm. Example of micro-scale structures that can be on chips of the present invention are wells, channels, dams, bricks, filters, scaffolds, electrodes, electromagnetic units, acoustic elements, or microfabricated pumps or valves. A variety of micro-scale structures are disclosed in U.S. patent application Ser. No. 09/679,024, having attorney docket number 471842000400, entitled “Apparatuses Containing Multiple Active Force Generating Elements and Uses Thereof” filed Oct. 4, 2000, herein incorporated by reference in its entirety. Micro-scale structures that can, when energy, such as an electrical signal, is applied, generate physical forces useful in the present invention, can be referred to as “physical force-generating elements” “physical force elements”, “active force elements”, or “active elements”.

A variety of micro-scale structures are disclosed in U.S. patent application Ser. No. 09/679,024, having attorney docket number 471842000400, entitled “Apparatuses Containing Multiple Active Force Generating Elements and Uses Thereof” filed Oct. 4, 2000, herein incorporated by reference in its entirety. Micro-scale structures that can, when energy, such as an electrical signal, is applied, generate physical forces useful in the present invention, can be referred to as “physical force-generating elements”, “physical force elements”, “active force elements”, or “active elements”.

A “multiple force chip” or “multiforce chip” is a chip that generates physical force fields and that has at least two different types of built-in structures each of which is, in combination with an external power source, capable of generating one type of physical field. A full description of the multiple force chip is provided in U.S. application Ser. No. 09/679,024 having attorney docket number 471842000400, entitled “Apparatuses Containing Multiple Active Force Generating Elements and Uses Thereof” filed Oct. 4, 2000, herein incorporated by reference in its entirety.

“Acoustic forces” are the forces exerted, directly or indirectly on moieties (e.g., particles and/or molecules) by an acoustic wave field. Acoustic forces can be used for manipulating (e.g., trapping, moving, directing, handling) particles in fluid. Acoustic waves, both standing acoustic wave and traveling acoustic wave, can exert forces directly on moieties and such forces are called “acoustic radiation forces”. Acoustic wave may also exert forces on the fluid medium in which the moieties are placed, or suspended, or dissolved and result in so-called acoustic streaming. The acoustic streaming, in turn, will exert forces on the moieties placed, suspended or dissolved in such a fluid medium. In this case, the acoustic wave fields can exert forces on moieties in directly.

“Acoustic elements” are structures that can generate an acoustic wave field in response to a power signal. Preferred acoustic elements are piezoelectric transducers that can generate vibrational (mechanical) energy in response to applied AC voltages. The vibrational energy can be transferred to a fluid that is in proximity to the transducers, causing an acoustic force to be exerted on particles (such as, for example, cells) in the fluid. A description of acoustic forces and acoustic elements can be found in U.S. patent application Ser. No. 09/636,104, filed Aug. 10, 2000, incorporated by reference in its entirety.

“Piezoelectic transducers” are structures capable of generating an acoustic field in response to an electrical signal. Non-limiting examples of the piezoelectric transducers are ceramic disks (e.g. PZT, Lead Zirconium Titinate) covered on both surfaces with metal film electrodes, piezoelectric thin films (e.g. zinc-oxide).

“Mixing”, as used herein, means the use of physical forces to cause movement in a sample, solution, or mixture, such that components of the sample, solution, or mixture become interspersed. Preferred methods of mixing for use in the present invention include use of acoustic forces.

“Processing” refers to the preparation of a sample for analysis, and can comprise one or multiple steps or tasks. Generally a processing task serves to separate components of a sample, concentrate components of a sample, at least partially purify components of a sample, or structurally alter components of a sample (for example, by lysis or denaturation).

As used herein, “isolating” means separating a desirable sample component from other non-desirable components of a sample, such that preferably, at least 15%, more preferably at least 30%, even more preferably at least 50%, and further preferably, at least 80% of the desirable sample components present in the original sample are retained, and preferably at least 50%, more preferably at least 80%, even more preferably, at least 95%, and yet more preferably, at least 99%, of at least one nondesirable component of the original component is removed, from the final preparation.

“Enrich” means increase the concentration of a sample component of a sample relative to other sample components (which can be the result of reducing the concentration of other sample components), or increase the concentration of a sample component. For example, as used herein, “enriching” nucleated fetal cells from a blood sample means increasing the proportion of nucleated fetal cells to all cells in the blood sample, enriching cancer cells of a blood sample can mean increasing the concentration of cancer cells in the sample (for example, by reducing the sample volume) or reducing the concentration of other cellular components of the blood sample, and “enriching” cancer cells in a urine sample can mean increasing their concentration in the sample.

“Separation” is a process in which one or more components of a sample are spatially separated from one or more other components of a sample. A separation can be performed such that one or more sample components of interest is translocated to or retained in one or more areas of a separation apparatus and at least some of the remaining components are translocated away from the area or areas where the one or more sample components of interest are translocated to and/or retained in, or in which one or more sample components is retained in one or more areas and at least some or the remaining components are removed from the area or areas. Alternatively, one or more components of a sample can be translocated to and/or retained in one or more areas and one or more sample components can be removed from the area or areas. It is also possible to cause one or more sample components to be translocated to one or more areas and one or more sample components of interest or one or more components of a sample to be translocated to one or more other areas. Separations can be achieved through, for example, filtration, or the use of physical, chemical, electrical, or magnetic forces. Nonlimiting examples of forces that can be used in separations are gravity, mass flow, dielectrophoretic forces, traveling-wave dielectrophoretic forces, and electromagnetic forces.

“Separating a sample component from a (fluid) sample” means separating a sample component from other components of the original sample, or from components of the sample that are remaining after one or more processing steps. “Removing a sample component from a (fluid) sample” means removing a sample component from other components of the original sample, or from components of the sample that are remaining after one or more processing steps.

“Capture” is a type of separation in which one or more moieties or sample components is retained in or on one or more areas of a surface, chamber, chip, tube, or any vessel that contains a sample, where the remainder of the sample can be removed from that area.

An “assay” is a test performed on a sample or a component of a sample. An assay can test for the presence of a component, the amount or concentration of a component, the composition of a component, the activity of a component, etc. Assays that can be performed in conjunction with the compositions and methods of the present invention include, but are not limited to, immunocytochemical assays, interphase FISH (fluorescence in situ hybridization), karyotyping, immunological assays, biochemical assays, binding assays, cellular assays, genetic assays, gene expression assays and protein expression assays.

A “binding assay” is an assay that tests for the presence or concentration of an entity by detecting binding of the entity to a specific binding member, or that tests the ability of an entity to bind another entity, or tests the binding affinity of one entity for another entity. An entity can be an organic or inorganic molecule, a molecular complex that comprises, organic, inorganic, or a combination of organic and inorganic compounds, an organelle, a virus, or a cell. Binding assays can use detectable labels or signal generating systems that give rise to detectable signals in the presence of the bound entity. Standard binding assays include those that rely on nucleic acid hybridization to detect specific nucleic acid sequences, those that rely on antibody binding to entities, and those that rely on ligands binding to receptors.

A “biochemical assay” is an assay that tests for the presence, concentration, or activity of one or more components of a sample.

A “cellular assay” is an assay that tests for a cellular process, such as, but not limited to, a metabolic activity, a catabolic activity, an ion channel activity, an intracellular signaling activity, a receptor-linked signaling activity, a transcriptional activity, a translational activity, or a secretory activity.

A “genetic assay” is an assay that tests for the presence or sequence of a genetic element, where a genetic element can be any segment of a DNA or RNA molecule, including, but not limited to, a gene, a repetitive element, a transposable element, a regulatory element, a telomere, a centromere, or DNA or RNA of unknown function. As nonlimiting examples, genetic assays can be gene expression assays, PCR assays, karyotyping, or FISH. Genetic assays can use nucleic acid hybridization techniques, can comprise nucleic acid sequencing reactions, or can use one or more enzymes such as polymerases, as, for example a genetic assay based on PCR. A genetic assay can use one or more detectable labels, such as, but not limited to, fluorochromes, radioisotopes, or signal generating systems.

“Immunostaining” refers to staining of a specific antigen or structure by any method in which the stain (or stain-generating system) is complexed with a specific antibody.

“Polymerase chain reaction” or “PCR” refers to method for amplifying specific sequences of nucleotides (amplicon). PCR depends on the ability of a nucleic acid polymerase, preferably a thermostable one, to extend a primer on a template containing the amplicon. RT-PCR is a PCR based on a template (cDNA) generated from reverse transcription from mRNA prepared from a sample. Quantitative Reverse Transcription PCR (qRT-PCR) or the Real-Time RT-PCR is a RT-PCR in which the RT-PCR products for each sample in every cycle are quantified.

“FISH” or “fluorescence in situ hybridization” is an assay wherein a genetic marker can be localized to a chromosome by hybridization. Typically, to perform FISH, a nucleic acid probe that is fluorescently labeled is hybridized to interphase chromosomes that are prepared on a slide. The presence and location of a hybridizing probe can be visualized by fluorescence microscopy. The probe can also include an enzyme and be used in conjunction with a fluorescent enzyme substrate.

“Karyotyping” refers to the analysis of chromosomes that includes the presence and number of chromosomes of each type (for example, each of the 24 chromosomes of the human haplotype (chromosomes 1-22, X, and Y)), and the presence of morphological abnormalities in the chromosomes, such as, for example, translocations or deletions. Karyotyping typically involves performing a chromosome spread of a cell in metaphase. The chromosomes can then be visualized using, for example, but not limited to, stains or genetic probes to distinguish the specific chromosomes.

A “gene expression assay” (or “gene expression profiling assay”) is an assay that tests for the presence or quantity of one or more gene expression products, i.e. messenger RNAs. The one or more types of mRNAs can be assayed simultaneously on cells of the interest from a sample. For different applications, the number and/or the types of mRNA molecules to be assayed in the gene expression assays may be different.

A “protein expression assay” (or “protein expression profiling assay”) is an assay that tests for the presence or quantity of one or more proteins. One or more types of protein can be assayed simultaneously on the cells of the interest from a sample. For different applications, the number and/or the types of protein molecules to be assayed in the protein expression assays may be different.

“Histological examination” refers to the examination of cells using histochemical or stains or specific binding members (generally coupled to detectable labels) that can determine the type of cell, the expression of particular markers by the cell, or can reveal structural features of the cell (such as the nucleus, cytoskeleton, etc.) or the state or function of a cell. In general, cells can be prepared on slides and “stained” using dyes or specific binding members directly or indirectly bound to detectable labels, for histological examination. Examples of dyes that can be used in histological examination are nuclear stains, such as Hoechst stains, or cell viability stains, such as Trypan blue, or cellular structure stains such as Wright or Giemsa, enzyme activity benzidine for HRP to form visible precipitate. Examples of specific binding members that can be used in histological examination of fetal red blood cells are antibodies that specifically recognize fetal or embryonic hemoglobin.

An “electrode” is a structure of highly electrically conductive material. A highly conductive material is a material with a conductivity greater than that of surrounding structures or materials. Suitable highly electrically conductive materials include metals, such as gold, chromium, platinum, aluminum, and the like, and can also include nonmetals, such as carbon and conductive polymers. An electrode can be any shape, such as rectangular, circular, castellated, etc. Electrodes can also comprise doped semi-conductors, where a semi-conducting material is mixed with small amounts of other “impurity” materials. For example, phosphorous-doped silicon may be used as conductive materials for forming electrodes.

A “well” is a structure in a chip, with a lower surface surrounded on at least two sides by one or more walls that extend from the lower surface of the well or channel. The walls can extend upward from the lower surface of a well or channel at any angle or in any way. The walls can be of an irregular conformation, that is, they may extend upward in a sigmoidal or otherwise curved or multi-angled fashion. The lower surface of the well or channel can be at the same level as the upper surface of a chip or higher than the upper surface of a chip, or lower than the upper surface of a chip, such that the well is a depression in the surface of a chip. The sides or walls of a well or channel can comprise materials other than those that make up the lower surface of a chip.

A “channel” is a structure in a chip with a lower surface and at least two walls that extend upward from the lower surface of the channel, and in which the length of two opposite walls is greater than the distance between the two opposite walls. A channel therefore allows for flow of a fluid along its internal length. A channel can be covered (a “tunnel”) or open.

A “pore” is an opening in a surface, such as a filter of the present invention, that provides fluid communication between one side of the surface and the other. A pore can be of any size and of any shape, but preferably a pore is of a size and shape that restricts passage of at least one insoluble sample component from one side of a filter to the other side of a filter based on the size, shape, deformability, binding affinity and/or binding specificity (or lack thereof), of the sample component.

A “slot” is an opening in a surface, such as a filter of the present invention. The slot length is longer than its width (slot length and slot width refer to the slots dimensions in the plane or the surface of the filter into which the sample components will go through, and slot depth refers to the thickness of the filter). The term “slot” therefore describes the shape of a pore, which will in some cases be approximately rectangular, ellipsoid, or that of a quadrilateral or parallelogram.

“Bricks” are structures that can be built into or onto a surface that can restrict the passage of sample components between bricks. The design and use of one type of bricks (called “obstacles”) on a chip is described in U.S. Pat. No. 5,837,115 issued Nov. 17, 1998 to Austin et al., herein incorporated by reference in its entirety.

A “dam” is a structure that can be built onto the lower surface of a chamber that extends upward toward the upper surface of a chamber leaving a space of defined width between the top of the dam and the top of the chamber. Preferably, the width of the space between the top of the dam and the upper wall of the chamber is such that fluid sample can pass through the space, but at least one sample component is unable to pass through the space based on its size, shape, or deformability (or lack thereof). The design and use of one type of dam structure on a chip is described in U.S. Pat. No. 5,928,880 issued Jul. 27, 1999 to Wilding et al., herein incorporated by reference in its entirety.

“Continuous flow” means that fluid is pumped or injected into a chamber of the present invention continuously during the separation process. This allows for components of a sample that are not selectively retained in a chamber to be flushed out of the chamber during the separation process.

“Binding partner” refers to any substances that both bind to the moieties with desired affinity or specificity and are manipulatable with the desired physical force(s). Non-limiting examples of the binding partners include microparticles.

A “microparticle” is a structure of any shape and of any composition that is manipulatable by desired physical force(s). The microparticles used in the methods could have a dimension from about 0.01 micron to about ten centimeters. Preferably, the microparticles used in the methods have a dimension from about 0.1 micron to about several hundred microns. Such particles or microparticles can be comprised of any suitable material, such as glass or ceramics, and/or one or more polymers, such as, for example, nylon, polytetrafluoroethylene (TEFLON™), polystyrene, polyacrylamide, sepaharose, agarose, cellulose, cellulose derivatives, or dextran, and/or can comprise metals. Examples of microparticles include, but are not limited to, magnetic beads, magnetic particles, plastic particles, ceramic particles, carbon particles, polystyrene microbeads, glass beads, hollow glass spheres, metal particles, particles of complex compositions, microfabricated free-standing microstructures, etc. The examples of microfabricated free-standing microstructures may include those described in “Design of asynchronous dielectric micromotors” by Hagedorn et al., in Journal of Electrostatics, Volume: 33, Pages 159-185 (1994). Particles of complex compositions refer to the particles that comprise or consists of multiple compositional elements, for example, a metallic sphere covered with a thin layer of non-conducting polymer film.

“A preparation of microparticles” is a composition that comprises microparticles of one or more types and can optionally include at least one other compound, molecule, structure, solution, reagent, particle, or chemical entity. For example, a preparation of microparticles can be a suspension of microparticles in a buffer, and can optionally include specific binding members, enzymes, inert particles, surfactants, ligands, detergents, etc.

As used herein, the term “substantially anti-parallel” and “substantially opposite” are understood to mean “approximately anti-parallel” and “approximately opposite”, respectively, such as within about 30°, preferably within about 20°, more preferably within about 10°, and most preferably within about 5° or less of being perfectly anti-parallel or opposite.

As used herein, the term “engaged” refers to any mode of mechanical or physical attachment, interlocking, mating, binding, or coupling, such that members that are said to be “engaged” do not come apart or detach from one another without some positive effort, application of energy, or the like.

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other technical terms used herein have their ordinary meaning in the art that they are used, as exemplified by a variety of technical dictionaries.

Introduction

The present invention recognizes that analysis of complex fluids, such as biological fluid samples, can be confounded by many sample components that can interfere with the analysis. Sample analysis can be even more problematic when the target of the analysis is a rare cell type: for example, when the target cells are fetal cells present in maternal blood or malignant cells present in the blood or urine of a patient. In processing such samples, it is often necessary to both “debulk” the sample, by reducing the volume to a manageable level, and to enrich the population of rare cells that are the target of analysis (see, e.g., U.S. Pat. Nos. 6,949,355 and 7,166,443; U.S. Patent Publication Nos. 2006/0252054, 2007/0202536, 2008/0057505 and 2008/0206757). Procedures for the processing of fluid samples are often time consuming and inefficient. In some aspects, the present invention provides efficient methods and automated systems for the separation of a target component from fluid samples.

As a non-limiting introduction to the breadth of the present invention, the present invention includes several general and useful aspects, including:

1) a filtration chamber comprising a microfabricated filter enclosed in a housing, wherein the filtration chamber comprises an antechamber and a post-filtration subchamber, and the fluid flow path in the antechamber is substantially opposite to the fluid flow path in the post-filtration subchamber;

2) a filtration chamber comprising a microfabricated filter enclosed in a housing, wherein the surface of said filter and/or the inner surface of said housing are modified by vapor deposition, sublimation, vapor-phase surface reaction, or particle sputtering to produce a uniform coating;

3) a filtration chamber comprising a microfabricated filter enclosed in a housing, wherein the surface of said filter and/or the inner surface of said housing are modified by a metal nitride, a metal halide, a Parylene or derivative thereof, a polytetrafluoroethylene (PTFE), a Teflon-AF or a perfluorocarbon;

4) a cartridge comprising a filtration chamber disclosed herein;

5) an automated filtration unit for separating a target component in a fluid sample, comprising a filtration chamber disclosed herein;

6) an automated system for separating and analyzing a target component in a fluid sample, comprising an automated filtration unit disclosed herein and an analysis apparatus connected to the filtration unit;

7) a method for separating a target component in a fluid sample, comprising: a) dispensing a fluid sample into the filtration chamber disclosed herein; and b) providing a fluid flow of the fluid sample through the filtration chamber, wherein the target component of the fluid sample is retained by or passes through the filter;

8) a method of separating a target component in a fluid sample using the automated filtration unit disclosed herein, comprising: a) dispensing the fluid sample into the filtration chamber; and b) providing a fluid flow of the fluid sample through the filtration chamber, wherein the target component of the fluid sample is retained by or flows through the filter; and

9) a method of enriching and analyzing a component in a fluid sample using the automated system disclosed herein, comprising: a) dispensing the fluid sample into the filtration chamber; b) providing a fluid flow of the fluid sample through the antechamber of the filtration chamber and a fluid flow of a solution through the post-filtration subchamber of the filtration chamber, wherein the target component of the fluid sample is retained in the antechamber and non-target components flow through the filter into the post-filtration subchamber; c) labeling the target component; and d) analyzing the labeled target component using the analysis apparatus.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

### I Filtration Chamber

In one aspect, the present invention provides a filtration chamber comprising a microfabricated filter enclosed in a housing. In embodiments in which a filtration chamber of the present invention comprises one or more microfabricated filters that are internal to the chamber, the filter or filters can divide the chamber into subchambers. Where a filtration chamber comprises a single internal microfabricated filter, for example, the filtration chamber can comprise a prefiltration “antechamber”, or where appropriate, “upper subchamber” and a “post-filtration subchamber”, or, where appropriate, “lower subchamber”. In other cases, a microfabricated filter can form a wall of a filtration chamber, and during filtration, filterable sample components exit the chamber via the filter.

In some embodiments of the present invention, a filtration chamber of the present invention has at least one port that allows for the introduction of a sample into the chamber, and conduits can transport sample to and from a filtration chamber of the present invention. When fluid flow commences, sample components that flow through one or more filters can flow into one or more areas of the chamber and then out of the chamber through conduits, and, preferably but optionally, from the conduits into a vessel, such as a waste vessel. The filtration chamber can also optionally have one or more additional ports for the additions of one or more reagents, solutions, or buffers. Throughout this description, it is understood that the inflow ports or outflow ports may be used with flow in the direction opposite to their named function.

In some embodiments, the filtration chamber may comprise an additional filter, or where appropriate, a “suprafilter”. In some embodiments, the suprafilter, between the antechamber and the suprachamber, may be any filter sufficiently rigid to maintain its flatness in slow flow conditions and be produced by any method that results in holes or slots with openings smaller than 5 microns. The suprafilter may further divide the antechamber or post-filtration subchamber. In some embodiments, the filtration antechamber may comprise an inflow port, an outflow port, and an additional inflow port, further where the additional inflow port is separated from the antechamber by another microfabricated filter thereby creating a suprachamber.

A filtration chamber of the present invention can comprise one or more fluid-impermeable materials, such as but not limited to, metals, polymers, plastics, ceramics, glass, silicon, or silicon dioxide. Preferably, a filtration chamber of the present invention has a volumetric capacity of from about 0.01 milliliters to about ten liters, more preferably from about 0.2 milliliters to about two liters. In some preferred embodiments of the present invention, a filtration chamber can have a volume of from about 1 milliliter to about 80 milliliters.

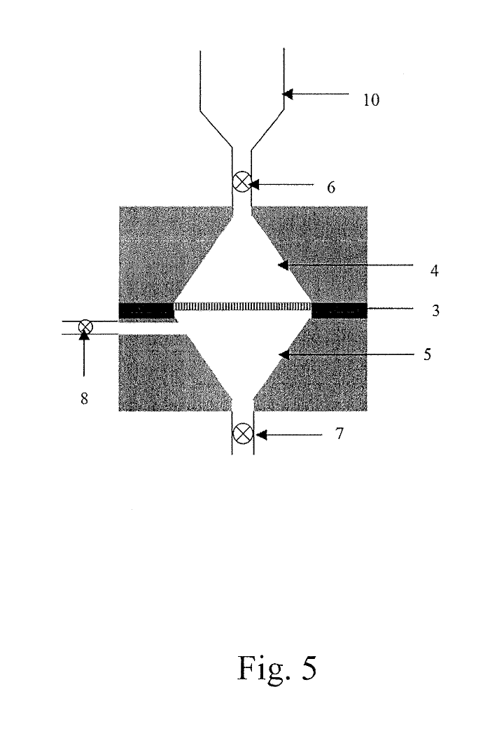

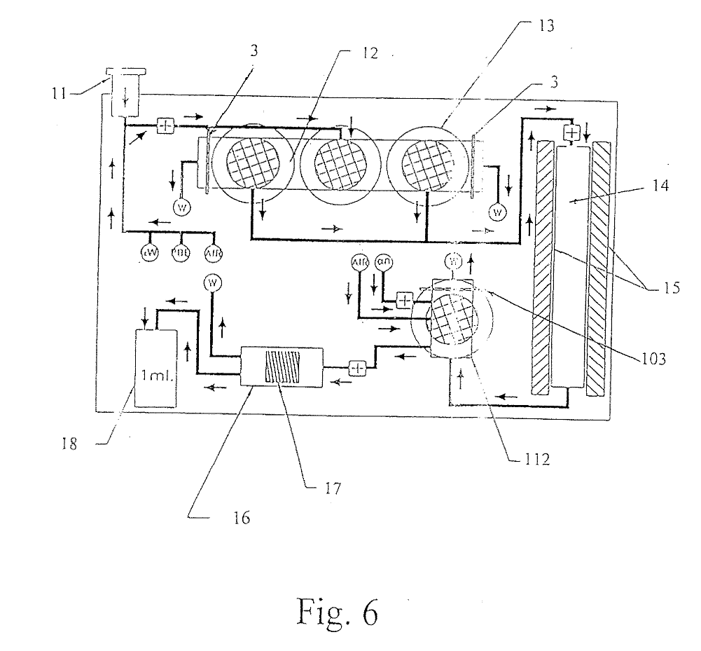

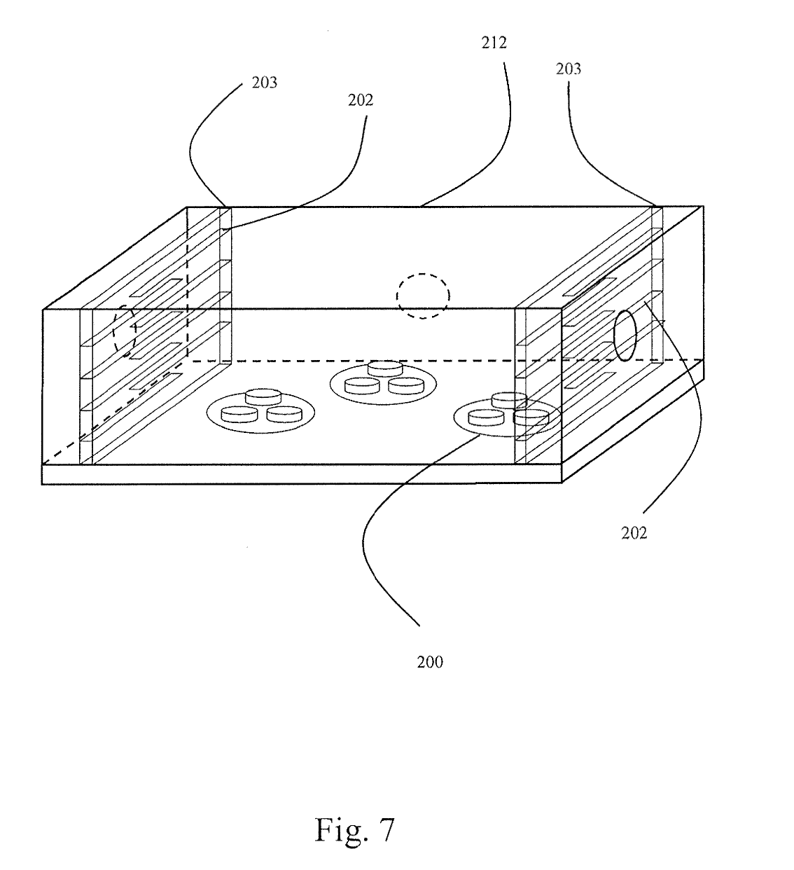

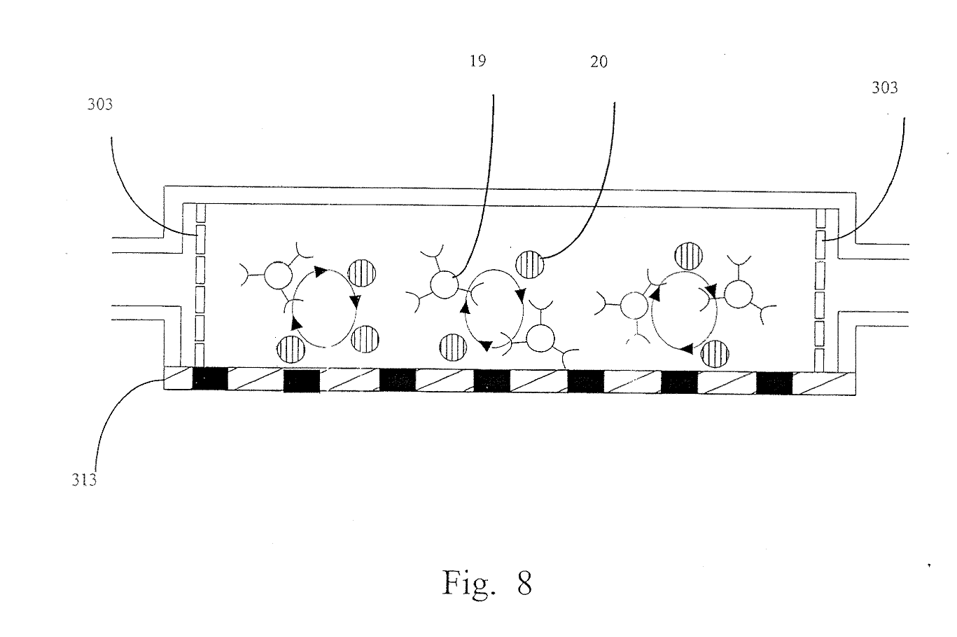

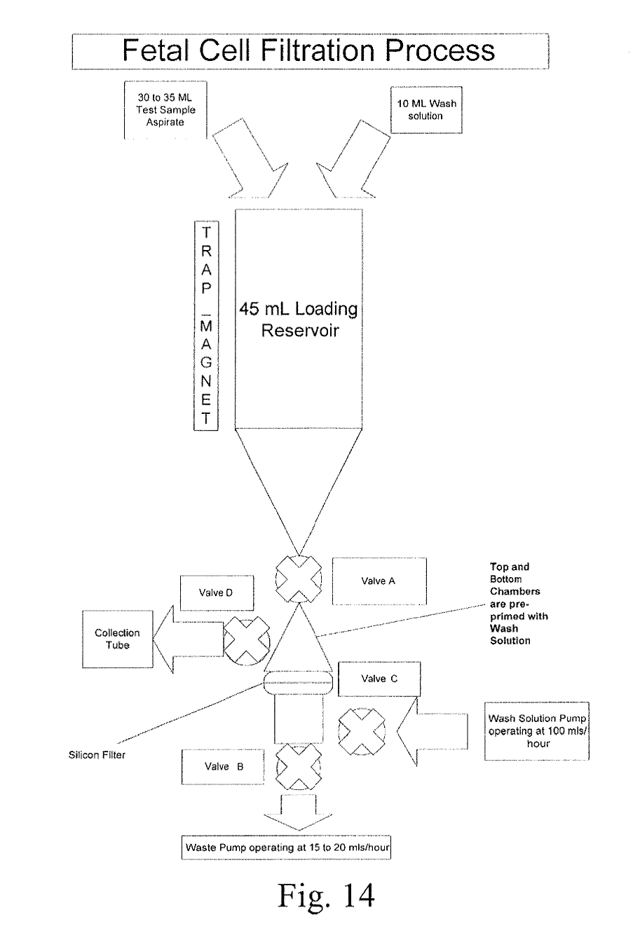

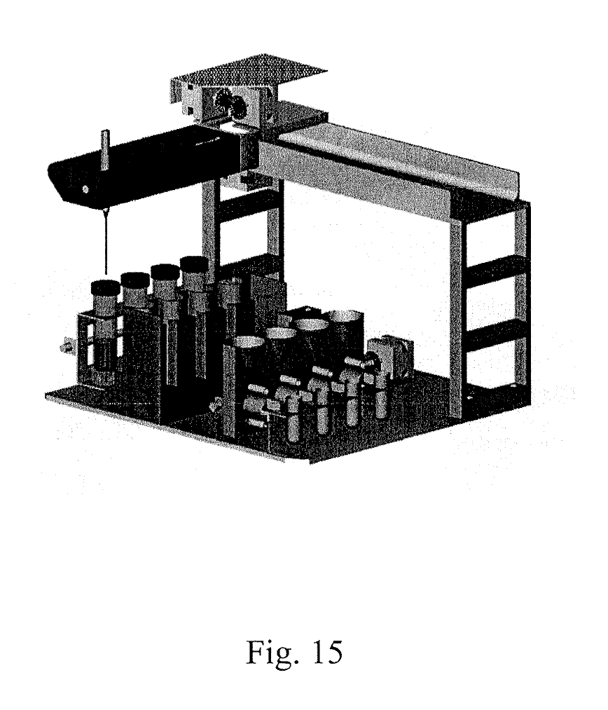

A filtration chamber of the present invention can comprise or engage any number of filters. In one preferred embodiment of the present invention, a filtration chamber comprises one filter (see, for example FIG. 5 and FIG. 14). In another preferred embodiment of the present invention, a filtration chamber comprises more than one filter, such as the chamber exemplified in FIG. 6 and FIG. 7. Various filter chamber configurations are possible. For example, it is within the scope of the present invention to have a filtration chamber in which one or more walls of the filter chamber comprises a microfabricated filter. It is also within the scope of the present invention to have a filtration chamber in which a filter chamber engages one or more filters. In this case, the filters can be permanently engaged with the chamber, or can be removable (for example, they can be inserted into slots or tracks provided on the chamber). A filter can be provided as a wall of a chamber, or internal to a chamber, and filters can optionally be provided in tandem for sequential filtering. Where filters are inserted into a chamber, they are inserted to form a tight seal with the walls of a chamber, such that during the filtration operation, fluid flow through the chamber (from one side of a filter to the other) must be through the pores of the filter.

In a preferred embodiment of the present invention, a filtration chamber of, for example, approximately one centimeter by one centimeter by 0.2 to ten centimeters in dimensions can have one or more filters comprising from four to 1,000,000 slots, preferably from 100 to 250,000 slots. In this preferred embodiment, the slots are preferably of rectangular shape, with a slot length of from about 0.1 to about 1,000 microns, and slot width is preferably from about 0.1 to about 100 microns, depending on the application.

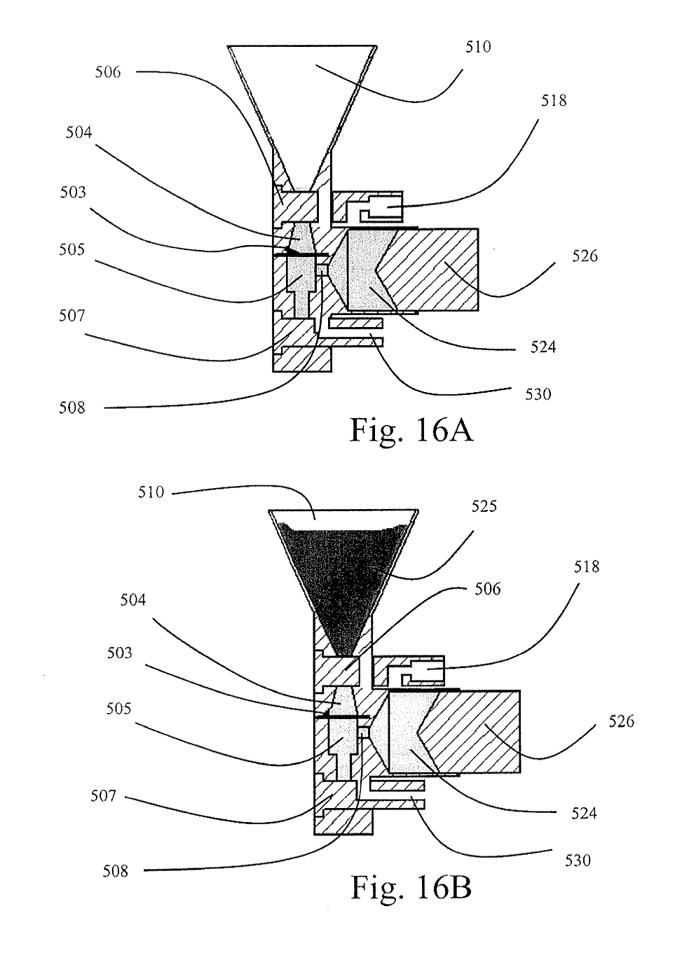

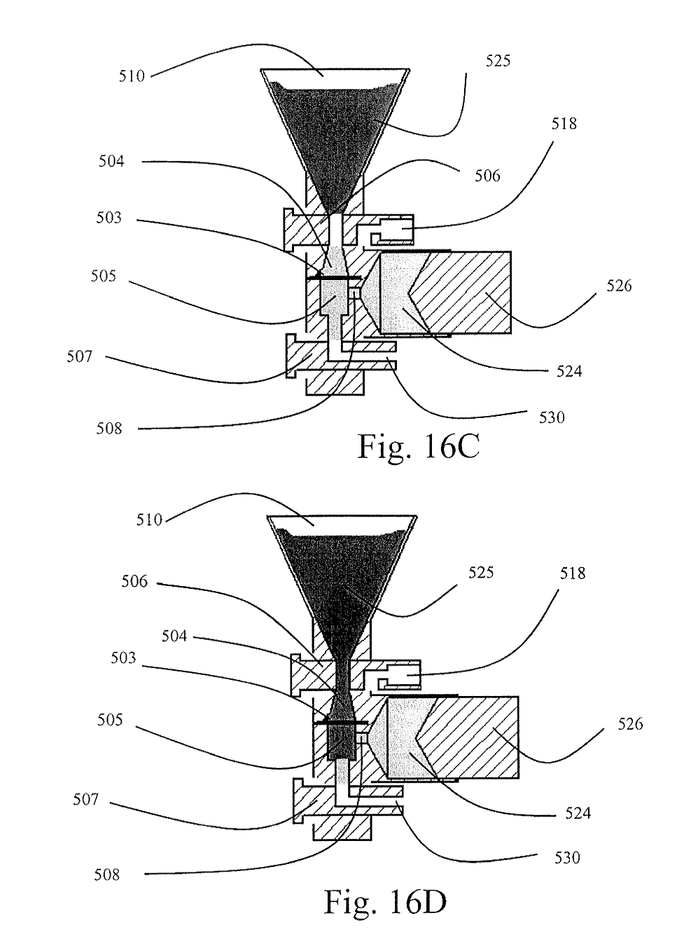

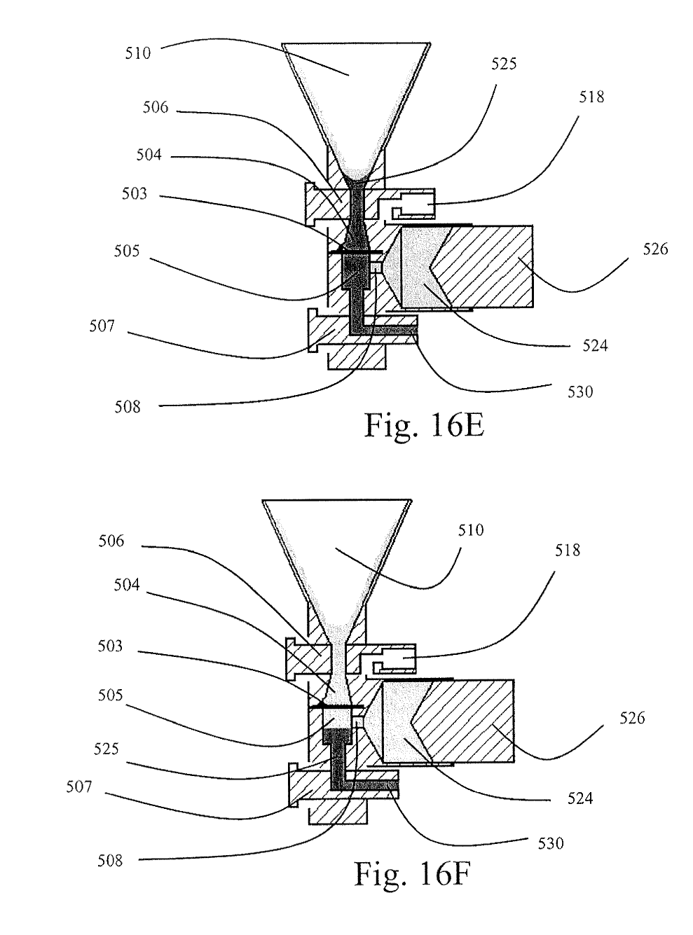

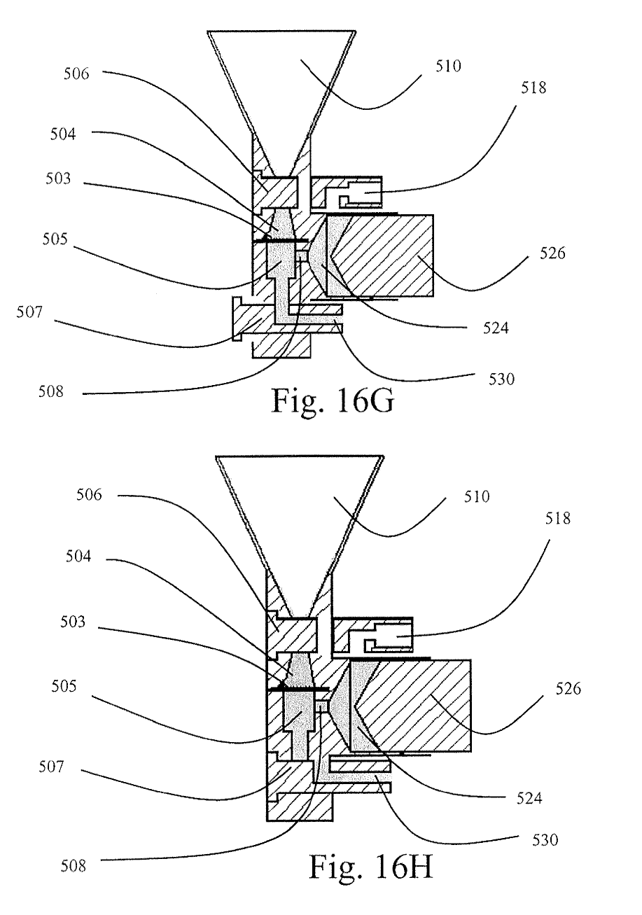

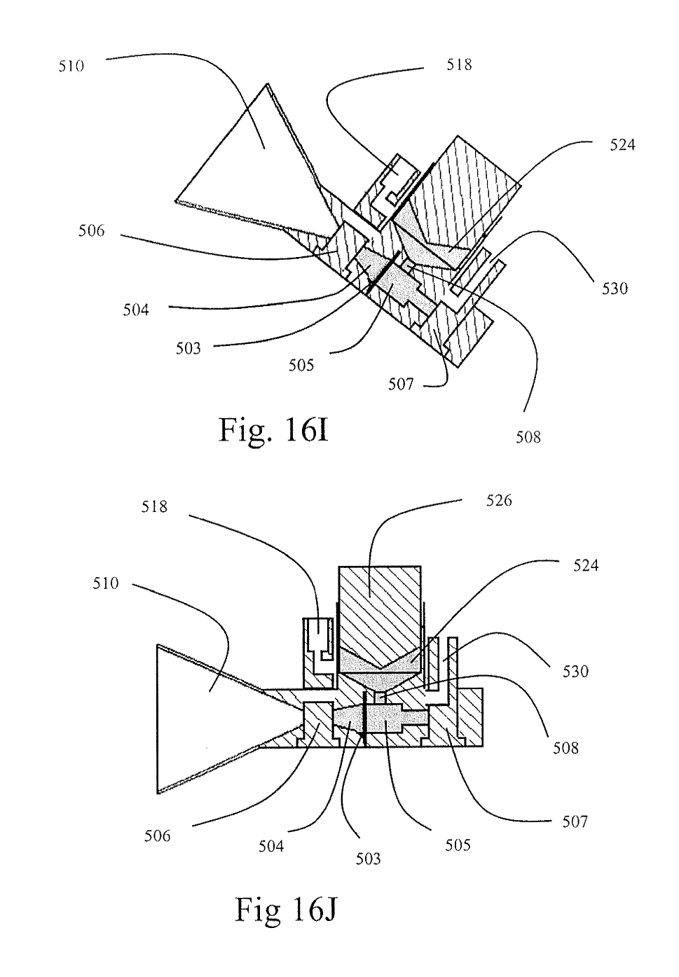

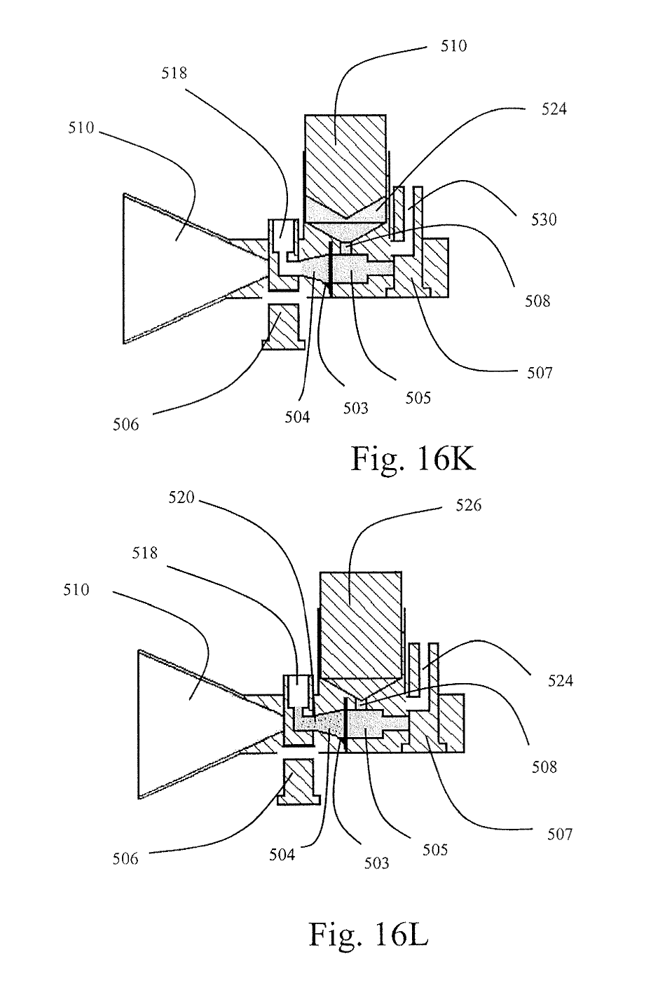

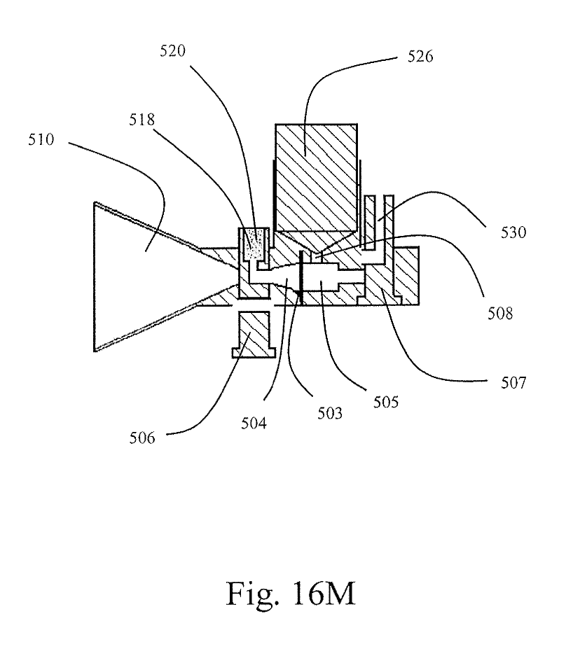

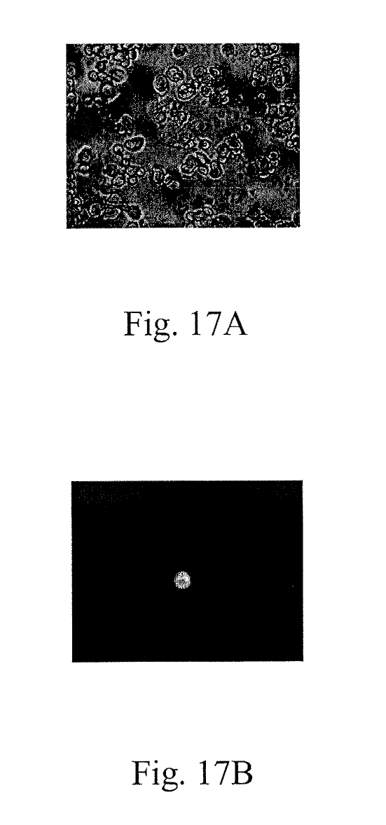

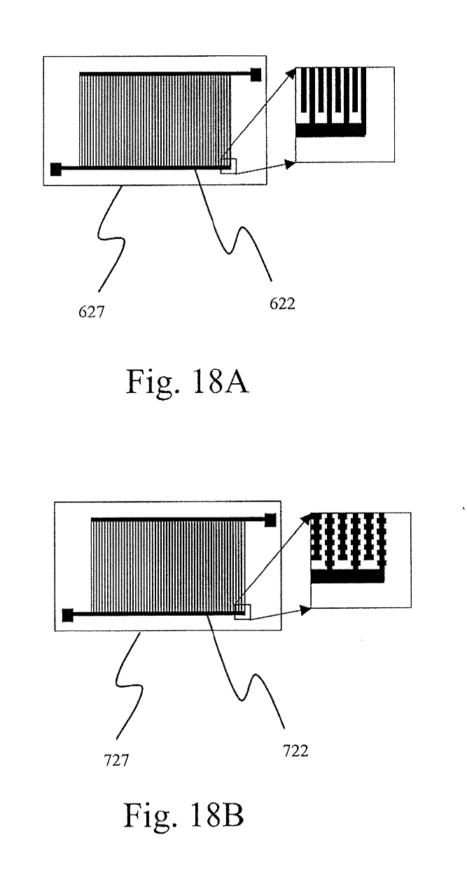

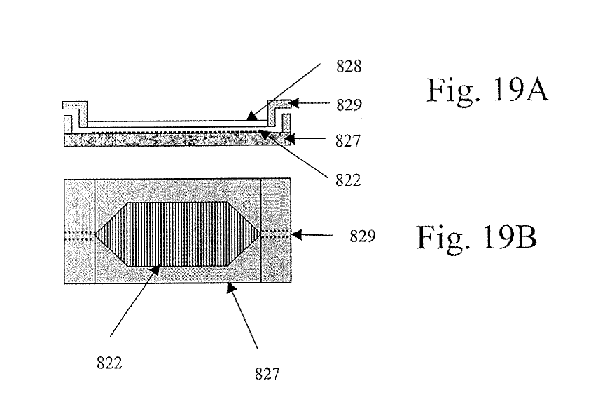

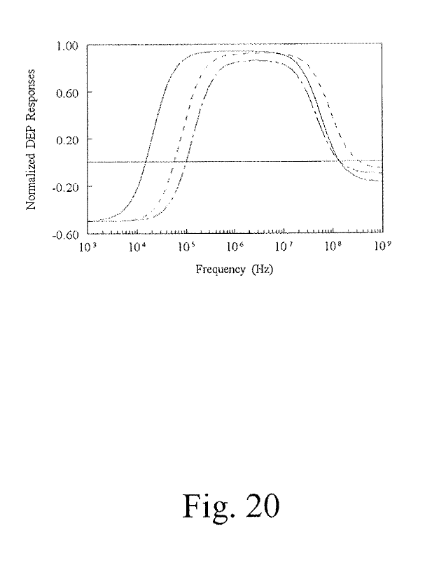

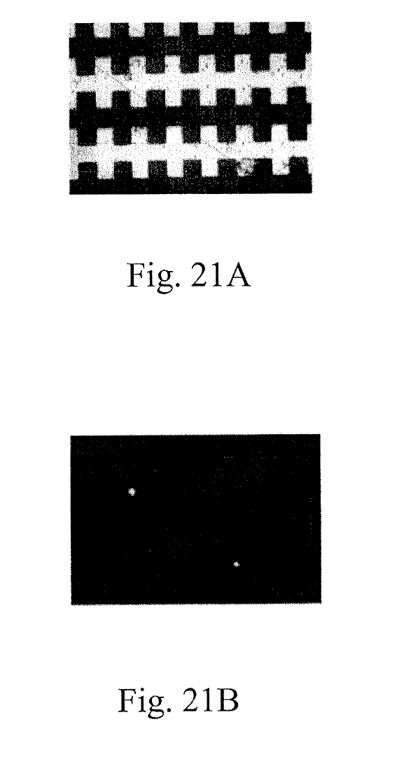

Preferably, slots can allow for the passage of mature red blood cells (lacking nuclei) through the channels and thus out of the chamber, while not or minimally allowing cells having a greater diameter or shape (for example but not limited to, nucleated cells such as white blood cells and nucleated red blood cells) to exit the chamber. A filtration chamber that can allow the removal of red blood cells by fluid flow through the chamber, while retaining other cells of a blood sample, is illustrated in FIG. 7, FIG. 14, and FIG. 16. For example, for removing matured red blood cells from nucleated RBCs and white blood cells, slot widths between 2.5 and 6.0 microns, more preferably between 2.2 and 4.0 microns, could be used. Slot length could vary between, for example, 20 and 200 microns. Slot depth (i.e., filter membrane thickness) can vary between 40 and 100 microns. The slot width between 2.0 and 4.0 microns would allow the double-discoid-shaped RBCs to go through the slots while primarily retaining the nucleated RBCs and WBCs with diameters or shapes larger than 7 micron.

Anti-Parallel Flow

In some embodiments, a filtration chamber of the present invention may be configured to allow parallel or anti-parallel fluid flow in the antechamber and the post-filtration subchamber. The antechamber may have two ports, an inflow port and an out flow port. The post-filtration subchamber may have two ports, an inflow port and an outflow port. The ports may be arranged in such a way that fluid flows in the antechamber and in the post-filtration subchamber are substantially opposite, or anti-parallel, of each other. The inflow port of the antechamber may be used to dispense a fluid sample, such as a blood sample, a cell suspension, or the like, into the filtration chamber.

In some embodiments, the device has a single antechamber with two ports for inflow and outflow, one on either side of the one or more filters, such that blood samples can flow through the antechamber. For example, blood samples can be pumped through the antechamber to fill the chamber. In preferred embodiments in which one opening comprises a reservoir at its end, particles such as cells and compounds can optionally be added via the reservoir. In the alternative, either particles, compounds, or both can be added to the antechamber at an opening that is not connected to a reservoir. In some embodiments, the antechamber may comprise more than one inflow and/or outflow ports. For example, an additional inflow port may be used for provide inflow of a solution for rinsing, or provide a fluidic force to push components of a fluid sample across the filter. In embodiments wherein a suprafilter is included to divide the antechamber into a suprachamber and an antechamber, the additional inflow port may provide fluid flow to the suprafilter.

In some preferred embodiments, the post-filtration subchamber is also a single flow-through channel, with an opening at one end for the introduction of solutions, and an opening at the other end for outflow of solutions. In some embodiments, the post-filtration subchamber may comprise more than one inflow and/or outflow ports. For example, multiple outflow ports in the post-filtration subchamber may be used to collect different filtration components based on the size, shape, deformability, binding affinity and/or binding specificity of the components.

In some embodiments, the fluid flow in the antechamber and the post-filtration subchamber may be such that a negative pressure may be created to draw components or cells through the filter. In some embodiments, the outflow from the bottom chamber is greater than the inflow into the bottom chamber such that a portion of the fluid sample traversing the antechamber may be drawn into the post-filtration subchamber such that the red blood cells and platelets will be separated from the white blood cells and other nucleated cells that will be retained in the antechamber by the filter. In some embodiments, the outflow fluid may contain fewer cells than the inflow fluid.

In some embodiments, the fluid flow of the antechamber and post-filtration subchamber may be configured so that they have different flow rates. It is contemplated that the difference in the fluid flow in the antechamber and post-filtration subchamber may create a fluid force across the filter between the antechamber and post-filtration subchamber. The flow rate of the fluid in the antechamber and post-filtration subchamber may be controlled by a pressure control unit, such as a pump, at the inflow and/or outflow ports. In some embodiments, the pressure control unit may be adjusted by an automatic control system, such as a computer running an algorithm.

The filtration chamber may include one or more surface contours to affect the flow of a sample, a solution such as wash or elution solution or both. For example contours may deflect, disperse or direct a sample to assist in the spreading of the sample along the filter. Alternatively, contours may deflect, disperse or direct a wash solution such that the wash solution washes the chamber or filter with greater efficiency. Such surface contours may be in any appropriate configuration. The contours may include surfaces that project generally toward the chip or may project generally away from the chip. They may generally encircle the filter. Contours may include but are not limited to projections, recessed portions, slots, deflection structures such as ball-like portions, bubbles (formed from e.g. air, detergent, or polymers), and the like. Contours such as two or more slots may be configured generally parallel to one another yet generally angled when viewing the chamber upright to direct flow in a generally spiraled path.

In some embodiments, the outflow port of the antechamber may be connected to a collection chamber, wherein the target components of the fluid sample, such as nucleated cells from a blood sample, or cancerous cells from a cell suspension, may be collected after unwanted components have been separated by filtration.

In some embodiments, the filtration chamber of the present invention may be formed by two housing parts, for example, a top housing part and a bottom housing part, which may reversibly engage to form the filtration chamber that encloses the filter. The housing parts may be bound together using any suitable methods, such as but not limited to, laser bonding, adhesive material, or the like. The bottom housing part can be in the form of a tray or tank, and preferably has at least one inlet and at least one outlet for allowing buffer to flow through the chamber.

Surface Treatment or Modification

In some embodiments, the present invention provides treatment or modifications to the surface of a microfabricated filter and/or the inner surface of a housing that encloses the microfabricated filter to improve its filtering efficiency. In some embodiments, the surface treatment produces a uniform coating of the filter and the housing. In some embodiments, one or both surfaces of the filter is treated or coated or modified to increase its filtering efficiency. The surface modifications may facilitate the filtration of components of the fluid sample across the filter, or reduce blocking of the slots on the filter by components of the fluid sample, such as cells, cell debris, protein aggregates, lipids, or the like. In some embodiments, one or both surfaces of the filter is treated or modified to reduce the possibility of sample components (such as but not limited to cells) interacting with or adhering to the filter.

The surface of the filter and/or the inner surface of the housing may be modified by a metal nitride, a metal halide, a Parylene, a polytetrafluoroethylene (PTFE), a Teflon-AF or a perfluorocarbon. In some embodiments, the perfluorocarbon may be in liquid form. In some embodiments, the perfluorocarbon may be 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane, which may be in liquid form. In some embodiments, the perfluorocarbon may be covalently bound to the surface. The surface modification of the filter and/or inner surface of the housing may be via vapor deposition, sublimation, vapor-phase surface reaction, or particle sputtering to produce a uniform coating.

A filter and/or housing can be physically or chemically treated, for example, to alter its surface properties (e.g., hydrophobic, hydrophilic). For example, vapor deposition, sublimation, vapor-phase surface reaction, or particle sputtering are some of the methods that can be used to treat or modify the surface of a filter and/or housing. Any suitable vapor deposition methods can be used, e.g., physical vapor deposition, plasma-enhanced chemical vapor deposition, chemical vapor deposition, etc. Suitable materials for physical vapor deposition, chemical vapor deposition, plasma-enhanced chemical vapor deposition or particle sputtering may include, but are not limited to, a metal nitride or a metal halide, such as titanium nitride, silicon nitride, zinc nitride, indium nitride, boron nitride, Parylene or a derivative thereof, such as Parylene, Parylene-N, Parylene-D, Parylene AF-4, Parylene SF, and Parylene HT. Polytetrafluoroethylene (PTFE) or Teflon-AF can also be used for chemical vapor deposition.

For example, a filter and/or housing can be heated or treated with plasma in chamber with a low nitrogen or ammonia or nitrous gas or other gases or any combination or sequence of these, modified to silicon nitride or can be treated with at least one acid or at least one base, to apply the desired surface charge and species. For example, a glass or silica filter and/or housing can be heated in a nitrogen or argon environment to remove oxide from the surface of the filter and/or housing. Heating times and temperatures can vary depending on the filter and/or housing material and the degree of reaction desired. In one example, a glass filter and/or housing can be heated to a temperature of from about 200 to 1200 degrees Celsius for from about thirty minutes to twenty-four hours.

In another example, a filter and/or housing can be treated with one or more acids or one or more bases to increase the electropositivity of the filter surface. In preferred embodiments, a filter and/or housing that comprises glass or silica is treated with at least one acid.

An acid used in treating a filter and/or housing of the present invention can be any acid. As nonlimiting examples, the acid can be formic acid, oxalic acid, ascorbic acid. The acid can be of a concentration about 0.1 N or greater, and preferably is about 0.5 N or higher in concentration, and more preferably is greater than about 1 N in concentration. For example, the concentration of acid preferably is from about 1 N to about 10 N. The incubation time can be from one minute to days, but preferably is from about 5 minutes to about 2 hours.

Optimal concentrations and incubation times for treating a microfabricated filter and/or housing to increase its hydrophilicity can be determined empirically. The microfabricated filter and/or housing can be placed in a solution of acid for any length of time, preferably for more than one minute, and more preferably for more than about five minutes. Acid treatment can be done under any non-freezing and non-boiling temperature, preferably at a temperature greater than or equal to room temperature.

Alternatively a reducing agent may be used in place of an acid or in addition to an acid or in any sequence with an acid, such as, but not limited to, hydrazine, lithium aluminum hydride, borohydrides, sulfites, phosphites, dithiothreitol, iron-containing compounds such as iron(II) sulfate. The reducing solution can be of a concentration of about 0.01 M or greater, and preferably is greater than about 0.05 M, and more preferably greater than about 0.1 M in concentration. The microfabricated filter and/or housing can be placed in a reducing solution for any length of time, preferably for more than one minute, and more preferably for more than about five minutes. Treatment can be done under any non-frozen and non-boiling temperature, preferably at a temperature greater than or equal to room temperature.

The effectiveness of a physical or chemical treatment in increasing the hydrophilicity of a filter and/or housing surface can be tested by measuring the spread of a drop of water placed on the surface of a treated and non-treated filter and/or housing, where increased spreading of a drop of uniform volume indicates increased hydrophilicity of a surface (FIG. 5). The effectiveness of a filter and/or housing treatment can also be tested by incubating a treated filter and/or housing with cells or biological samples to determine the degree of sample component adhesion to the treated filter and/or housing.

In another embodiment, the surface of a filter and/or housing, such as but not limited to a polymeric filter and/or housing, can chemically treated to alter the surface properties of the filter and/or housing. For example, the surface of a glass, silica, or polymeric filter and/or housing can be derivatized by any of various chemical treatments to add chemical groups that can decrease the interaction of sample components with the filter and/or housing surface.

One or more compounds can also be adsorbed onto or conjugated to the surface of a microfabricated filter and/or housing made of any suitable material, such as, for example, one or more metals, one or more ceramics, one or more polymers, glass, silica, silicon nitride, or combinations thereof. In preferred embodiments of the present invention, the surface or surfaces of a microfabricated filter and/or housing of the present invention is coated with a compound to increase the efficiency of filtration by reducing the interaction of sample components with the filter and/or housing surface.

For example, the surface of a filter and/or housing can be coated with a molecule, such as, but not limited to, a protein, peptide, or polymer, including naturally occurring or synthetic polymers. The material used to coat the filter and/or housing is preferably biocompatible, meaning it does not have deleterious effects on cells or other components of biological samples, such as proteins, nucleic acids, etc. Albumin proteins, such as bovine serum albumin (BSA) are examples of proteins that can be used to coat a microfabricated filter and/or housing of the present invention. Polymers used to coat a filter and/or housing can be any polymer that does not promote cell sticking to the filter and/or housing, for example, non-hydrophobic polymers such as, but not limited to, polyethylene glycol (PEG), polyvinylacetate (PVA), and polyvinylpyrrolidone (PVP), and a cellulose or cellulose-like derivative.

A filter and/or housing made of, for example, metal, ceramics, a polymer, glass, or silica can be coated with a compound by any feasible means, such as, for example, adsorption or chemical conjugation.

In many cases, it can be advantageous to surface-treat the filter and/or housing prior to coating with a compound or polymer. Surface treatment can increase the stability and uniformity of the coating. For example, a filter and/or housing can be treated with at least one acid or at least one base, or with at least one acid and at least one base, prior to coating the filter and/or housing with a compound or polymer. In preferred aspects of the present invention, a filter and/or housing made of a polymer, glass, or silica is treated with at least one acid and then incubated in a solution of the coating compound for a period of time ranging from minutes to days. For example, a glass filter and/or housing can be incubated in acid, rinsed with water, and then incubated in a solution of BSA, PEG, or PVP.

In some aspects of the present invention, it can be preferred to rinse the filter and/or housing, such as in water (for example, deionized water) or a buffered solution before acid or base treatment or treatment with an oxidizing agent, and, preferably again before coating the filter and/or housing with a compound or polymer. Where more than one type of treatment is performed on a microfabricated filter and/or housing, rinses can also be performed between treatments, for example, between treatment with an oxidizing agent and an acid, or between treatment with an acid and a base. A filter and/or housing can be rinsed in water or an aqueous solution that has a pH of between about 3.5 and about 10.5, and more preferably between about 5 and about 9. Non-limiting examples of suitable aqueous solutions for rinsing microfabricated filter and/or housing can include salt solutions (where salt solutions can range in concentration from the micromolar range to 5M or more), biological buffer solutions, cell media, or dilutions or combinations thereof. Rinsing can be performed for any length of time, for example from minutes to hours.

The concentration of a compound or polymer solution used to coat a filter and/or housing can vary from about 0.02% to 20% or more, and will depend in part on the compound used. The incubation in coating solution can be from minutes to days, and preferably is from about 10 minutes to two hours.

After coating, the filter and/or housing can be rinsed in water or a buffer.

The treatment methods of the present invention can also be applied to chips other than those that comprise pores for filtration. For example, chips that comprise metals, ceramics, one or more polymers, silicon, silicon dioxide, or glass can be physically or chemically treated using the methods of the present invention. Such chips can be used, for example, in separation, analysis, and detection devices in which biological species such as cells, organelles, complexes, or biomolecules (for example, nucleic acids, proteins, small molecules) are separated, detected, or analyzed. The treatment of the chip can enhance or reduce the interaction of the biological species with the chip surface, depending of the treatment used, the properties of the biological species being manipulated, and the nature of the manipulation. For example, a chip can be coated with a hydrophilic or hydrophobic polymer, depending on the biological species being manipulated and the nature of the manipulation. As a further example, coating the surface of the chip with a hydrophilic polymer (for example but not limited to coating the chip with PVP or PVA) may reduce or minimize the interaction between the surface of the chip and the cells.

Multiplexing

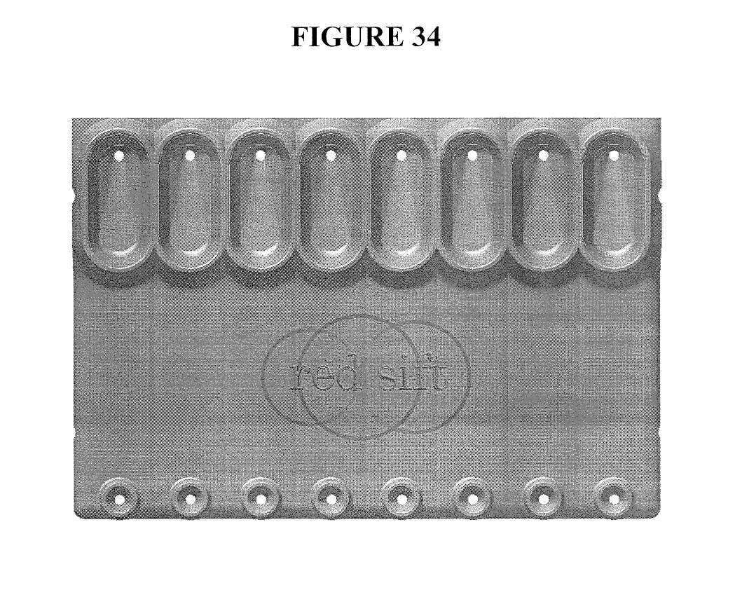

In some embodiments of the present invention, more than one filtration chambers may be combined in a multiplex configuration. For example, at least 2, 3, 4, 5, 6, 7, 8, 9, 10 or more filtration chambers may be combined. FIG. 34 shows an exemplary embodiment wherein eight filtration chambers are combined. In some embodiments, each filtration chamber of the multiplex configuration is independent of each other, i.e., is not in fluidic connection with other filtration chambers in the multiplex configuration. In some embodiments, some or all of the filtration chambers of the multiplex configuration may be in fluidic connection with each other. For example, some or all of the filtration chambers may have a common housing, or may be connected with each other by a fluidic channel or conduit.

The filtration chambers in a multiplex configuration may be arranged side by side, as shown in FIG. 34, or arranged in linear fashion, or both. The filtration chambers in a multiplex configuration may be arranged in the same orientation, or opposite orientation, or a combination thereof. In some embodiments, at least two filtration chambers operate in tandem further wherein the slots of the filters within each filtration chamber are of different widths where the filtration chambers are arranged in order of increasing slot widths.

In some embodiments, at least two filtration chambers are arranged in tandem and where subsequent filtration chambers comprise filters of increasing slot widths. In some embodiments, the filter contains slot widths of increasing size along the fluidic path and further where a suprafiltration chamber exists and the post-filtration chamber contains multiple partitions that direct the fluidic flow out through one outflow port per partition. In some embodiments, the outflow ports from each partition segment of the post-filtration chamber may be aligned with and deposit its outflow directly into individual wells of a multiwell drug screening plate with wells spaced every 2.25 mm or every 4.5 mm or every 9 mm or every 18 mm.

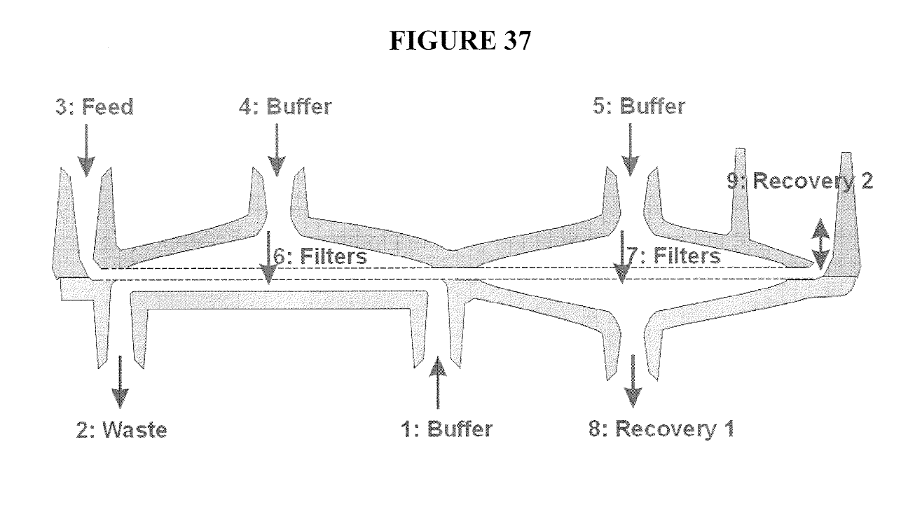

FIG. 37 illustrates another embodiment of a multiplex configuration. In this configuration, the two filtration chambers are in fluidic connection through the antechamber between a suprafilter and a microfabricated filter. The filters of the filtration chambers may have different slot sizes, so that different components may be recovered in recovery areas 1 and 2.

Automated Filtration Unit

In some embodiments, a filtration chamber of the present invention is part of a filtration unit which comprise a means to control fluid flow through the filtration chamber. Any suitable mechanisms may be used to control the fluid flow in the filtration chamber, such as fluidic pumps, valves, conduits, channels, or the like. In some embodiments, a control algorithm, for example, a computer program, may be used to control the fluid flow. Fluid flow in both the antechamber and post-filtration subchamber may be controlled by the control algorithm.

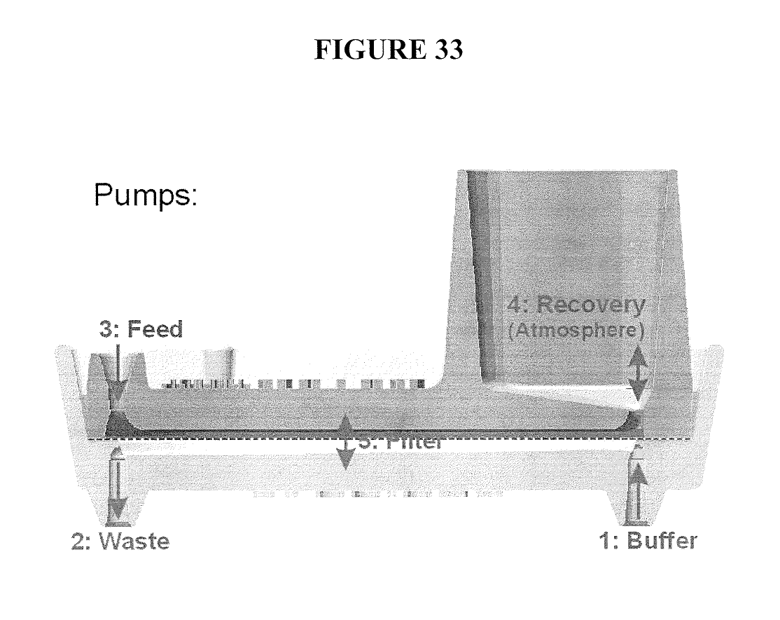

In embodiments wherein the fluid flows in the antechamber and post-filtration subchamber are substantially anti-parallel, such as depicted in FIG. 33, multiple fluidic pumps may be used to separately control the flow rate in the antechamber and post-filtration subchamber. A feed pump (3) may be used to control the fluid flow rate in the antechamber, and a buffer pump (1) and a waste pump (2) may be used to control the fluid flow rate in the post-filtration subchamber.

In some embodiments, the fluid flow of the antechamber and post-filtration subchamber may be configured so that they have different flow rates. It is contemplated that the difference in the fluid flow in the antechamber and post-filtration subchamber may create a fluid force across the filter between the antechamber and post-filtration subchamber.

In some embodiments, the fluid flow in the antechamber and the post-filtration subchamber may be such that a negative pressure (5) may be created to draw components or cells through the filter. In some embodiments, the outflow from the bottom chamber is greater than the inflow into the bottom chamber such that a portion of the fluid sample traversing the antechamber may be drawn into the post-filtration subchamber such that the red blood cells and platelets will be separated from the white blood cells and other nucleated cells that will be retained in the antechamber by the filter. In some embodiments, the outflow fluid may contain fewer cells than the inflow fluid.

For example, one preferred filtration unit of the present invention, depicted in FIG. 5, comprises a valve-controlled inlet for the addition of sample (valve A (6)), a valve connected to a conduit through which negative pressure is applied for the filtration of the sample (valve B (7)), and a valve controlling the flow of wash buffer into the filtration chamber for washing the chamber (valve C (8)). In some embodiments of the present invention, a filtration unit can comprise valves that can optionally be under automatic control that allow sample to enter the chamber, waste to exit the chamber, and negative pressure to provide fluid flow for filtration.

In order to transfer a solution or supernatant to the filtration chamber, a needle (but not limited to stated object) can be used. A needle may be connected to the container (e.g. tubing or chamber) that can hold a volume. The needle may collect cells from a tube containing a solution and dispense the solution into another chamber using a device to push or pull a solution (e.g. pump or syringe).

In some embodiments, the inflow port of the antechamber may be connected to a column, so that a specific binding member for an unwanted component of the sample fluid may be immobilized on a solid surface in the column. For example, a lectin, a receptor ligand or an antibody may be immobilized in the column to remove red blood cells, white blood cells, or platelets from a blood sample.

Automated System for Separating and Analyzing Components of a Fluid Sample

Further provided herein is an automated system for separating and analyzing a target components of a fluid sample, which comprise a filtration chamber in fluid connection with an apparatus for analyzing the target component separated by the filtration chamber. In some embodiments, the antechamber of the filtration chamber may be directly connected to the apparatus, so that the target component, such as nucleated cells or rare cells retained by the filter, may directly enter the apparatus for analysis. The outflow port of the antechamber, or the collection chamber, may also be connected to the apparatus, for example, a flow cytometer, so that the separated component may be directly analyzed without further manipulation. In some embodiments, the target component by be labeled before the analysis.

Filter Comprising Electrodes

In some preferred embodiments, traveling-wave dielectrophoretic forces can be generated by electrodes built onto a chip that is part of a filtration chamber, and can be used to move sample components such as cells away from a filter. In this case, the microelectrodes are fabricated onto the filter surfaces and the electrodes are arranged so that the traveling wave dielectrophoresis can cause the sample components such as cells to move on the electrode plane or the filter surface through which the filtration process occur. A full description of the traveling wave dielectrophoresis is provided in U.S. application Ser. No. 09/679,024 having attorney docket number 471842000400, entitled “Apparatuses Containing Multiple Active Force Generating Elements and Uses Thereof” filed Oct. 4, 2000, herein incorporated by reference in its entirety.

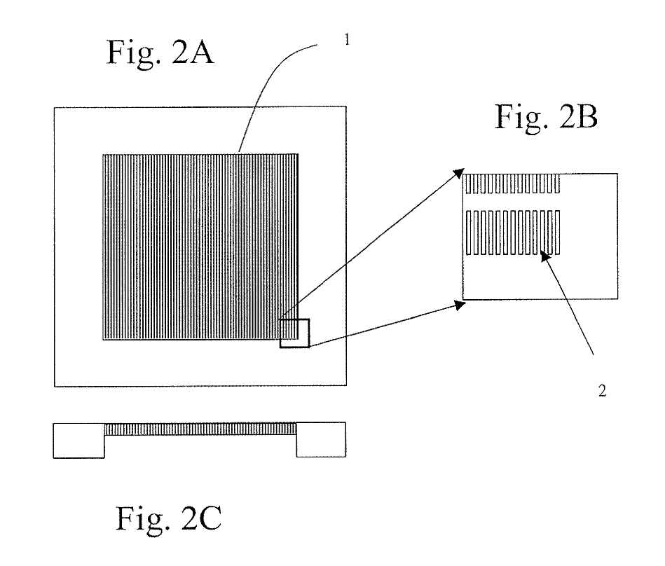

In one embodiment of the filters, interdigitated microelectrodes are fabricated onto the filter surfaces such as those shown in FIG. 2 or described in “Novel dielectrophoresis-based device of the selective retention of viable cells in cell culture media” by Docoslis et al, in Biotechnology and Bioengineering, Vol. 54, No. 3, pages 239-250, 1997, and in the U.S. Pat. No. 5,626,734, issued to Docoslis et al. on May 7, 1997. For this embodiment, the negative dielectrophoretic forces generated by the electrodes can repel the sample components such as the cells from the filter surface or from the filter slots so that the collected cells on the filters are not clogging the filters during the filtration process. Where traveling-wave dielectrophoresis or negative dielectrophoresis is used to enhance filtration, electrode elements can be energized periodically throughout the filtration process, during periods when fluid flow is halted or greatly reduced.

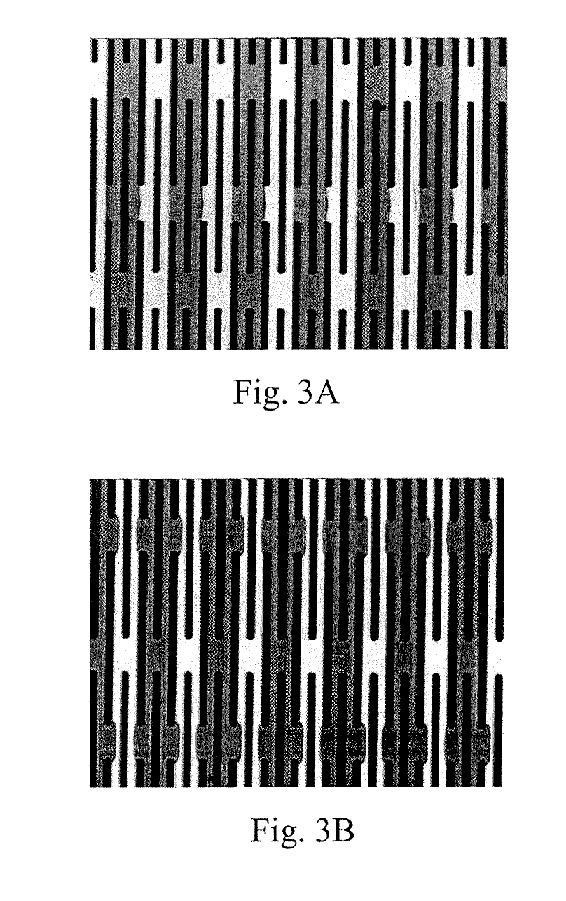

Filters having slots in the micron range that incorporate electrodes that can generate dielectrophoretic forces are illustrated in FIGS. 3 (A and B). For example, filters have been made in which the interdigitated electrodes of 18 micron width and 18 micron gaps were fabricated on the filters, which were made on silicon substrates. Individual filter slots were of rectangular shape with dimensions of 100 micron (length) by 2-3.8 micron (width). Each filter had a unique slot size (e.g. length by width: 100 micron by 2.4 micron, 100 micron by 3 micron, 100 micron by 3.8 micron). Along the length direction, the gap between the adjacent filter slots was 20 micron. Along the width direction, the adjacent slots were not aligned; instead, they were offset. The offset distance between neighboring columns of the filter slots were 50 micron or 30 micron, alternatively. The filter slots were positioned with respect to the electrodes so that the slot center lines along the length direction were aligned with the center line of the electrodes, or the electrode edges, or the center line of the gaps between the electrodes.

Electrodes may also be positioned on the housing of the filtration chamber that encloses the filter. In some embodiments, electrodes may be positioned in an antechamber and/or a post-filtration subchamber. The electrodes may be positioned in relation to the filter in such a way that dielectrophoretic forces are generated around the filter slots. In some embodiments, the dielectrophoretic forces may keep the cells or other sample components away from the filter slots or filter surface.

The following discussion and references can provide a framework for the design and use of electrodes to facilitate filtration by translocating sample components, such as nonfilterable cells, away from a filter:

Dielectrophoresis refers to the movement of polarized particles in a non-uniform AC electrical field. When a particle is placed in an electrical field, if the dielectric properties of the particle and its surrounding medium are different, the particle will experience dielectric polarization. Thus, electrical charges are induced at the particle/medium interface. If the applied field is non-uniform, then the interaction between the non-uniform field and the induced polarization charges will produce net force acting on the particle to cause particle motion towards the region of strong or weak field intensity. The net force acting on the particle is called dielectrophoretic force and the particle motion is dielectrophoresis. Dielectrophoretic force depends on the dielectric properties of the particles, particle surrounding medium, the frequency of the applied electrical field and the field distribution.

Traveling-wave dielectrophoresis is similar to dielectrophoresis in which the traveling-electric field interacts with the field-induced polarization and generates electrical forces acting on the particles. Particles are caused to move either with or against the direction of the traveling field. Traveling-wave dielectrophoretic forces depend on the dielectric properties of the particles and their suspending medium, the frequency and the magnitude of the traveling-field. The theory for dielectrophoresis and traveling-wave dielectrophoresis and the use of dielectrophoresis for manipulation and processing of microparticles may be found in various publications (e.g., “Non-uniform Spatial Distributions of Both the Magnitude and Phase of AC Electric Fields determine Dielectrophoretic Forces by Wang et al., in Biochim Biophys Acta Vol. 1243, 1995, pages 185-194”, “Dielectrophoretic Manipulation of Particles” by Wang et al, in IEEE Transaction on Industry Applications, Vol. 33, No. 3, May/June, 1997, pages 660-669, “Electrokinetic behavior of colloidal particles in traveling electric fields: studies using yeast cells” by Huang et al, in J. Phys. D: Appl. Phys., Vol. 26, pages 1528-1535, “Positioning and manipulation of cells and microparticles using miniaturized electric field traps and traveling waves” By Fuhr et al., in Sensors and Materials. Vol. 7: pages 131-146, “Dielectrophoretic manipulation of cells using spiral electrodes” by Wang, X-B. et al., in Biophys. J. Volume 72, pages 1887-1899, 1997, “Separation of human breast cancer cells from blood by differential dielectric affinity” by Becker et al, in Proc. Natl. Acad. Sci., Vol., 92, January 1995, pages 860-864). The manipulation of microparticles with dielectrophoresis and traveling wave dielectrophoresis include concentration/aggregation, trapping, repulsion, linear or other directed motion, levitation, or separation of particles. Particles may be focused, enriched and trapped in specific regions of the electrode reaction chamber. Particles may be separated into different subpopulations over a microscopic scale. Relevant to the filtration methods of the present invention, particles may be transported over certain distances. The electrical field distribution necessary for specific particle manipulation depends on the dimension and geometry of microelectrode structures and may be designed using dielectrophoresis theory and electrical field simulation methods.

The dielectrophoretic force F_{DEP z} acting on a particle of radius r subjected to a non-uniform electrical field can be given by

F_{DEP z}=2π∈ₘr³χ_{DEP}∇Eᵣₘₛ²·\vec{a}_{z}

where Eᵣₘₛ is the RMS value of the field strength, ∈ₘ is the dielectric permitivity of the medium. χ_{DEP} is the particle dielectric polarization factor or dielectrophoresis polarization factor, given by

$${χ}_{DEP} = Re ( \frac{{ɛ}_{p}^{*} - {ɛ}_{m}^{*}}{{ɛ}_{p}^{*} + 2 {ɛ}_{m}^{*}} ) ,$$

“Re” refers to the real part of the “complex number”. The symbol

$${ɛ}_{x}^{*} = {ɛ}_{x} - j \frac{{σ}_{x}}{2 π f}$$

is the complex permitivity (of the particle x=p, and the medium x=m). The parameters ∈ₚ and σₚ are the effective permitivity and conductivity of the particle, respectively. These parameters may be frequency dependent. For example, a typical biological cell will have frequency dependent, effective conductivity and permitivity, at least, because of cytoplasm membrane polarization.

The above equation for the dielectrophoretic force can also be written as

F_{DEP z}=2π∈ₘr³χ_{DEP}V²p(z)\vec{a}_{z}

where p(z) is the square-field distribution for a unit-voltage excitation (V=1 V) on the electrodes, V is the applied voltage.

There are generally two types of dielectrophoresis, positive dielectrophoresis and negative dielectrophoresis. In positive dielectrophoresis, particles are moved by dielectrophoresis forces towards the strong field regions. In negative dielectrophoresis, particles are moved by dielectrophoresis forces towards weak field regions. Whether particles exhibit positive and negative dielectrophoresis depends on whether particles are more or less polarizable than the surrounding medium. In the filtration methods of the present invention, electrode patterns on one or more filters of a filtration chamber can be designed to cause sample components such as cells to exhibit negative dielectrophoresis, resulting in sample components such as cells being repelled away from the electrodes on the filter surfaces.

Traveling-wave DEP force refers to the force that is generated on particles or molecules due to a traveling-wave electric field. A traveling-wave electric field is characterized by the non-uniform distribution of the phase values of AC electric field components.

Here we analyze the traveling-wave DEP force for an ideal traveling-wave field. The dielectrophoretic force F_{DEP} acting on a particle of radius r subjected to a traveling-wave electrical field E_{TWD}=E cos(2π(ft−z/λ₀)\vec{a}ₓ (i.e., a x-direction field is traveling along the z-direction) is given by

F_{TWD}=−2π∈ₘr³ζ_{TWD}E²·\vec{a}_{z}

where E is the magnitude of the field strength, ∈ₘ is the dielectric permittivity of the medium. ζ_{TWD} is the particle polarization factor, given by

$${ζ}_{TWD} = Im ( \frac{{ɛ}_{p}^{*} - {ɛ}_{m}^{*}}{{ɛ}_{p}^{*} + 2 {ɛ}_{m}^{*}} ) ,$$

“Im” refers to the imaginary part of the “complex number”. The symbol

$${ɛ}_{x}^{*} = {ɛ}_{x} - j \frac{{σ}_{x}}{2 π f}$$

is the complex permittivity (of the particle x=p, and the medium x=m). The parameters ∈ₚ and σₚ are the effective permittivity and conductivity of the particle, respectively. These parameters may be frequency dependent.

Particles such as biological cells having different dielectric property (as defined by permittivity and conductivity) will experience different dielectrophoretic forces. For traveling-wave DEP manipulation of particles (including biological cells), traveling-wave DEP forces acting on a particle of 10 micron in diameter can vary somewhere between 0.01 and 10000 pN.

A traveling wave electric field can be established by applying appropriate AC signals to the microelectrodes appropriately arranged on a chip. For generating a traveling-wave-electric field, it is necessary to apply at least three types of electrical signals each having a different phase value. An example to produce a traveling wave electric field is to use four phase-quardrature signals (0, 90, 180 and 270 degrees) to energize four linear, parallel electrodes patterned on the chip surfaces. Such four electrodes form a basic, repeating unit. Depending on the applications, there may be more than two such units that are located next to each other. This will produce a traveling-electric field in the spaces above or near the electrodes. As long as electrode elements are arranged following certain spatially sequential orders, applying phase-sequenced signals will result in establishing traveling electrical fields in the region close to the electrodes.

Both dielectrophoretic and traveling-wave dielectrophoretic forces acting on particles depend on not only the field distributions (e.g., the magnitude, frequency and phase distribution of electrical field components; the modulation of the field for magnitude and/or frequency) but also the dielectric properties of the particles and the medium in which particles are suspended or placed. For dielectrophoresis, if particles are more polarizable than the medium (e.g., having larger conductivities and/or permittivities depending on the applied frequency), particles will experience positive dielectrophoretic forces and are directed towards the strong field regions. The particles which are less polarizable than the surrounding medium will experience negative dielectrophoretic forces and are directed towards the weak field regions. For traveling wave dielectrophoresis, particles may experience dielectrophoretic forces that drive them in the same direction as the field traveling direction or against it, dependent on the polarization factor ζ_{TWD}. The following papers provide basic theories and practices for dielectrophoresis and traveling-wave-dielectrophoresis: Huang, et al., J. Phys. D: Appl. Phys. 26:1528-1535 (1993); Wang, et al., Biochim. Biophys. Acta. 1243:185-194 (1995); Wang, et al., IEEE Trans. Ind. Appl. 33:660-669 (1997).

Filtration Chamber Comprising Active Chip

A filtration chamber can also preferably comprise or engage at least a portion of at least one active chip, where an active chip is a chip that uses applied physical forces to promote, enhance, or facilitate processing or desired biochemical reactions of a sample, or and to decrease or reduce any undesired effects that might otherwise occur to or in a sample. An active chip of a filtration chamber of the present invention preferably comprises acoustic elements, electrodes, or even electromagnetic elements. An active chip can be used to transmit a physical force that can prevent clogging of the slots or around the structures used to create a filter (for example, blocks, dams, or channels, slots etched into and through the filter substrate) by components of the sample that are too large to go through the pores or slots or openings, or become aggregated at the pores or slots or openings. For example, when an electric signal is applied, acoustic elements can cause mixing of the components within the chamber, thereby dislodging nonfilterable components from the slots or pores.

In an alternative embodiment, a pattern of electrodes on a chip can provide negative dielectrophoresis of sample components to move the nonfilterable components from the vicinity of the slots, channels, or openings around structures and allow access of filterable sample components to the slots or openings. Example of such electrode arrays fabricated onto a filter under a different operating mechanism of “dielectrophoretic-base selective retention” have been described in “Novel dielectrophoresis-based device of the selective retention of viable cells in cell culture media” by Docoslis et al, in Biotechnology and Bioengineering, Vol. 54, No. 3, pages 239-250, 1997, herein incorporated by reference and in the U.S. Pat. No. 5,626,734, issued to Docoslis et al on May 7, 1997, herein incorporated by reference. Active chips, including chips that can be used to mix samples by acoustic forces and chips that can be used to move moieties, including sample components, by dielectrophoretic forces, are described in U.S. application Ser. No. 09/636,104, filed Aug. 10, 2000, entitled “Methods for Manipulating Moieties in Microfluidic Systems”, U.S. provisional application 60/239,299, entitled “An Integrated Biochip System for Sample Preparation and Analysis”, filed Oct. 10, 2000, and U.S. application Ser. No. 09/686,737, filed Oct. 10, 2000 entitled “Compositions and Methods for Separation of Moieties on Chips”, all herein incorporated by reference.

The incorporation of electrodes that can be used for traveling wave dielectrophoresis on a filter of the present invention, as well as principles of dielectrophoresis and traveling wave dielectrophoresis, has been described herein in a previous description of microfabricated filters. Electrodes can also be incorporated onto active chips that are used in filtration chambers of the present invention to improve filtration efficiency.

A filtration chamber can also comprise a chip that comprises electromagnetic elements. Such electromagnetic elements can be used for the capture of sample components before or, preferably, after, filtering of the sample. Sample components can be captured after being bound to magnetic beads. The captured sample components can be either undesirable components to be retained in the chamber after the sample containing desirable components has already been removed from the chamber, or the captured sample components can be desirable components captured in the chamber after filtration.

An acoustic force chip can engage or be part of a filtration chamber, or one or more acoustic elements can be provided on one or more walls of a filtration chamber. Mixing of a sample by the activation of the acoustic force chip can occur during the filtration procedure. Preferably, a power supply is used to transmit an electric signal to the acoustic elements of one or more acoustic chips or one or more acoustic elements on one or more walls or a chamber. One or more acoustic elements can be active continuously throughout the filtration procedure, or can be activated for intervals (pulses) during the filtration procedure.

Sample components and, optionally, solutions or reagents added to the sample can be mixed by acoustic forces that act on both the fluid and the moieties, including, but not limited to, molecules, complexes, cells, and microparticles, in the chamber. Acoustic forces can cause mixing by acoustic streaming of fluid that occurs when acoustic elements, when energized by electrical signals generate mechanical vibrations that are transmitted into and through the fluid. In addition, acoustic energy can cause movement of sample components and/or reagents by generating acoustic waves that generate acoustic radiation forces on the sample components (moieties) or reagents themselves.

The following discussion and references can provide a framework for the design and use of acoustic elements to provide a mixing function:

Acoustic force refers to the force that is generated on moieties, e.g., particles and/or molecules, by an acoustic wave field. (It may also be termed acoustic radiation forces.) The acoustic forces can be used for manipulating, e.g., trapping, moving, directing, handling, mixing, particles in fluid. The use of the acoustic force in a standing ultrasound wave for particle manipulation has been demonstrated for concentrating erythrocytes (Yasuda et al, J. Acoust. Soc. Am., 102(1):642-645 (1997)), focusing micron-size polystyrene beads (0.3 to 10 micron in diameter, Yasuda and Kamakura, Appl. Phys. Lett, 71(13):1771-1773 (1997)), concentrating DNA molecules (Yasuda et al, J. Acoust. Soc. Am., 99(2):1248-1251, (1996)), batch and semi-continuous aggregation and sedimentation of cells (Pui et al, Biotechnol. Prog., 11:146-152 (1995)). By competing electrostatic and acoustic radiation forces, separation of polystyrene beads of different size and charges have been reported (Yasuda et al, J. Acoust. Soc. Am., 99(4):1965-1970 (1996); and Yasuda et al., Jpn. J. Appl. Phys., 35(1):3295-3299 (1996)). Furthermore, little or no damage or harming effect was observed when acoustic radiation force was used to manipulate mammalian cells, as characterized in terms of ion leakage (for erythrocytes, Yasuda et al, J. Acoust. Soc. Am., 102(1):642-645 (1997)) or antibody production (for hybridoma cells, Pui et al, Biotechnol. Prog., 11:146-152 (1995)).

An acoustic wave can be established by an acoustic transducer, e.g., piezoelectric ceramics such as PZT material. The piezoelectric transducers are made from “piezoelectric materials” that produce an electric field when exposed to a change in dimension caused by an imposed mechanical force (piezoelectric or generator effect). Conversely, an applied electric field will produce a mechanical stress (electrostrictive or motor effect) in the materials. They transform energy from mechanical to electrical and vice-versa. When AC voltages are applied to the piezoelectric transducers, the vibration occurs to the transducers and such vibration can be coupled into a fluid that is placed in the chamber comprising the piezoelectric transducers.

An acoustic chip can comprise acoustic transducers so that when AC signals at appropriate frequencies are applied to the electrodes on the acoustic transducers, the alternating mechanical stress is produced within the piezoelectric materials and is transmitted into the liquid solutions in the chamber. In a situation where the chamber is set up so that a standing acoustic wave is established along the direction (e.g.: z-axis) of wave propagation and reflection, the standing wave spatially varying along the z axis in a fluid can be expressed as:

Δp(z)=p₀ sin(kz)cos(ωt)

where Δp is acoustic pressure at z, p₀ is the acoustic pressure amplitude, k is the wave number (2π/λ, where λ is the wavelength), z is the distance from the pressure node, ω is the angular frequency, and t is the time. In one example, the standing-wave acoustic field may be generated by the superimposition of an acoustic wave generated from an acoustic transducer that forms a major surface of a chamber and the reflective wave from another major surface of the chamber that is positioned in parallel with the acoustic transducer and reflects the acoustic wave from the transducer. According to the theory developed by Yosioka and Kawasima (Acoustic Radiation Pressure on a Compressible Sphere by Yosioka K. and Kawasima Y. in Acustica, Volume 5, pages 167-173, 1955), the acoustic force F_{acoustic} acting on a spherical particle in the stationary standing wave field is given by

$${F}_{acoustic} = - \frac{4 π}{3} {r}^{3} k {E}_{acoustic} A sin ( 2 kz )$$

where r is the particle radius, E_{acoustic} is the average acoustic energy density, A is a constant given by

$$A = \frac{5 {ρ}_{p} - 2 {ρ}_{m}}{2 {ρ}_{p} + {ρ}_{m}} - \frac{{γ}_{p}}{{γ}_{m}}$$

where ρₘ and ρₚ are the density of the particle and the medium, γₘ and γₚ are the compressibility of the particle and medium, respectively. The compressibility of a material is the product of the density of the material and the velocity of acoustic-wave in the material. The compressibility is sometimes termed acoustic impedance. A is termed as the acoustic-polarization-factor.

When A>0, the particle moves towards the pressure node (z=0) of the standing wave.

When A<0, the particle moves away from the pressure node.

The acoustic radiation forces acting on particles depend on acoustic energy density distribution and on particle density and compressibility. Particles having different density and compressibility will experience different acoustic-radiation-forces when they are placed into the same standing acoustic wave field. For example, the acoustic radiation force acting on a particle of 10 micron in diameter can vary somewhere between <0.01 and >1000 pN, depending on the established acoustic energy density distribution.

The above analysis considers the acoustic radiation forces exerted on particles in a standing acoustic wave. Further analysis may be extended to the case of the acoustic radiation forces exerted on particles in a traveling-wave case. Generally, an acoustic wave field may consist of both standing and traveling-wave components. In such cases, particles in the chamber will experience acoustic radiation forces in the form other than those described by above equations. The following papers provide detailed analysis of acoustic radiation forces on spherical particles by traveling acoustic wave and standing acoustic waves: Yosioka et al., Acoustic Radiation Pressure on a Compressible Sphere. Acustica (1955) 5:167-173; and Hasegawa, Acoustic-Radiation force on a solid elastic sphere. J. Acoust. Soc. Am. (1969) 46:1139.

The acoustic radiation forces on particles may also be generated by various special cases of acoustic waves. For example, acoustic forces may be produced by a focused beam (“Acoustic radiation force on a small compressible sphere in a focused beam” by Wu and Du, J. Acoust. Soc. Am., 87:997-1003 (1990)), or by acoustic tweezers (“Acoustic tweezers” by Wu J. Acoust. Soc. Am., 89:2140-2143 (1991)).

Acoustic wave field established in a fluid can also induce a time-independent fluid flow, as termed acoustic streaming. Such fluid flow may also be utilized in biochip applications or microfluidic applications for transporting or pumping fluids. Furthermore, such acoustic-wave fluid flow may be exploited for manipulating molecules or particles in fluids. The acoustic streaming depends on acoustic field distributions and on fluid properties (“Nonlinear phenomena” by Rooney J. A. in “Methods of Experimental Physics: Ultrasonics, Editor: P. D. Edmonds”, Chapter 6.4, pages 319-327, Academic Press, 1981; “Acoustic Streaming” by Nyborg W. L. M. in “Physical Acoustics, Vol. II-Part B, Properties of Polymers and Nonlinear Acoustics”, Chapter 11, pages 265-330, 1965).

Thus, one or more active chips, such as one or more acoustic force chips, can also be used to promote mixing of reagents, solutions, or buffers, that can be added to a filtration chamber, before, during, or after the addition of a sample and the filtration process. For example, reagents, such as, but not limited to specific binding members that can aid in the removal of undesirable sample components, or in the capture of desirable sample components, can be added to a filtration chamber after the filtration process has been completed and the conduits have been closed off. The acoustic elements of the active chip can be used to promote mixing of one or more specific binding members with the sample whose volume has been reduced by filtration. One example is the mixing of sample components with magnetic beads that comprise antibodies that can bind particular cell types (for example, white blood cells, or fetal nucleated red blood cells) within the sample. The magnetic beads can be used to selectively remove or separate (capture) undesirable or desirable sample components, respectively, in subsequent steps of a method of the present invention. The acoustic elements can be activated for a continuous mixing period, or in pulses.

Microfabricated Filter

In one aspect, the present invention includes a microfabricated filter that comprises at least one tapered pore, where a pore is an opening in the filter. A pore can be of any shape and any dimensions. For example, a pore can be quadrilateral, rectangular, ellipsoid, or circular in shape, or of any other shape. A pore can have a diameter (or widest dimension) from about 0.1 micron to about 1000 microns, preferably from about 20 to about 200 microns, depending on the filtering application. Preferably, a pore is made during the machining of a filter, and is microetched or bored into the filter material that comprises a hard, fluid-impermeable material such as glass, silicon, ceramic, metal or hard plastic such as acrylic, polycarbonate, or polyimide. It is also possible to use a relatively non-hard surface for the filter that is supported on a hard solid support. Another aspect of this invention is to modify the material (for example but not limited to chemically or thermally modifying the material to silicon oxide or silicon nitride). Preferably, however, the filter comprises a hard material that is not deformable by the pressure (such as suction pressure) used in generating fluid flow through the filter.

A slot is a pore with a length that is greater than its width, where “length” and “width” are dimensions of the opening in the plane of the filter. (The “depth” of the slot corresponds to the thickness of the filter.) That is, “slot” describes the shape of the opening, which will in most cases be approximately rectangular or ellipsoid, but can also approximate a quadrilateral or parallelogram. In preferred embodiments of the present invention in which slot width is the critical dimension in determining which sample components flow through or are retained by the filter, the shape of the slot can vary at the ends (for example, be regular or irregular in shape, curved or angular), but preferably the long sides of the slot are a consistent distance from one another for most of the length of the slot, that distance being the slot width. Thus the long sides of a slot will be parallel or very nearly parallel, for most of the length of the slot.

Preferably, the filters used for filtration in the present invention are microfabricated or micro-machined filters so that the pores or the slots within a filter can achieve precise and uniform dimensions. Such precise and uniform pore or slot dimensions are a distinct advantage of the microfabricated or micro-machined filters of the present invention, in comparison with the conventional membrane filters made of materials such as nylon, polycarbonate, polyester, mixed cellulose ester, polytetrafluoroethylene, polyethersulfone, etc. In the filters of the present invention, individual pores are isolated, have similar or almost identical feature sizes, and are patterned on a filter. Such filters allow precise separation of particles based on their sizes and other properties.

The filtration area of a filter is determined by the area of the substrate comprising the pores. The filtration area for microfabricated filters of the present invention can be between about 0.01 mm² and about 0.1 m². Preferably, the filtration area is between about 0.25 mm² and about 25 cm², and more preferably is between about 0.5 mm² and about 10 cm². The large filtration areas allow the filters of the invention to process sample volumes from about 10 microliters to about 10 liters. The percent of the filtration area encompassed by pores can be from about 1% to about 70%, preferably is from about 10% to about 50%, and more preferably is from about 15 to about 40%. The filtration area of a microfabricated filter of the present invention can comprise any number of pores, and preferably comprises at least two pores, but more preferably the number of pores in the filtration area of a filter of the present invention ranges from about 4 to about 1,000,000, and even more preferably ranges from about 100 to about 250,000. The thickness of the filter in the filtration area can range from about 10 to about 500 microns, but is preferably in the range of between about 40 and about 100 microns.

The microfabricated filters of the present invention have slots or pores that are etched through the filter substrate itself. The pores or openings of the filters can be made by using microfabrication or micromachining techniques on substrate materials, including, but not limited to, silicon, silicon dioxide, ceramics, glass, polymers such as polyimide, polyamide, etc. Various fabrication methods, as known to those skilled in the art of microlithography and microfabrication (See, for example, Rai-Choudhury P. (Editor), Handbook of Microlithography, Micromachining and Microfabrication, Volume 2: Micromachining and microfabrication. SPIE Optical Engineering Press, Bellingham, Wash., USA (1997)), may be used. In many cases, standard microfabrication and micromachining methods and protocols may be involved. One example of suitable fabrication methods is photolithography involving single or multiple photomasks. The protocols in the microfabrication may include many basic steps, for example, photolithographic mask generation, deposition of photoresist, deposition of “sacrificial” material layers, photoresist patterning with masks and developers, or “sacrificial” material layer patterning. Pores can be made by etching into the substrate under certain masking process so that the regions that have been masked are not etched off and the regions that have not been mask-protected are etched off. The etching method can be dry-etching such as deep RIE (reactive ion etching), laser ablation, or can be wet etching involving the use of wet chemicals. The material may be grown by a positive method whereby the slots or pores appear as the substrate material is depositioned or grown around them or the material may be grown around a masking resist that when removed will produce the holes or slots.

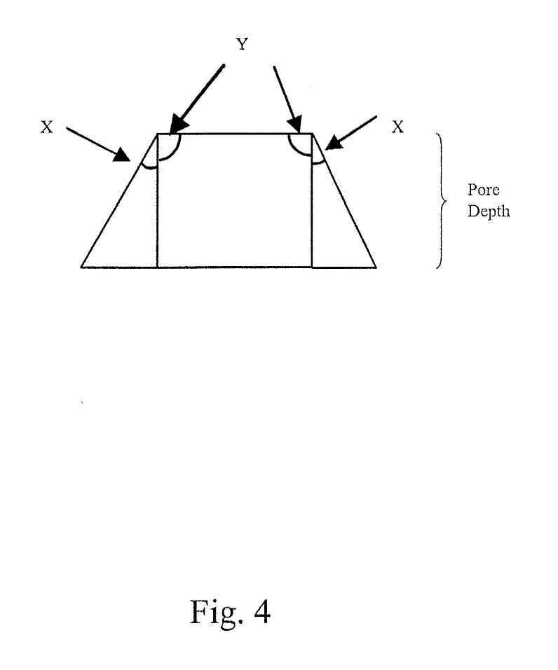

Preferably, appropriate microfabrication or micromachining techniques are chosen to achieve a desired aspect ratio for the filter pores. The aspect ratio refers to the ratio of the slot depth (corresponding to the thickness of the filter in the region of the pores) to the slot width or slot length. The fabrication of filter slots with higher aspect ratios (i.e., greater slot depth) may involve deep etching methods. Many fabrication methods, such as deep RIE, useful for the fabrication of MEMS (microelectronic mechanical systems) devices can be used or employed in making the microfabricated filters. The resulting pores can, as a result of the high aspect ratio and the etching method, have a slight tapering, such that their openings are narrower on one side of the filter than the other. For example, in FIG. 4, the angle Y, of a hypothetical pore bored straight through the filter substrate is 90 degrees, and the tapering angle X by which a tapered pore of a microfabricated filter of the present invention differs from the perpendicular is between about 0 degree and about 90 degrees, and preferably between 0.1 degrees and 45 degrees and most preferably between about 0.5 degrees and 10 degrees, depending on the thickness of the filter (pore depth).

The present invention includes microfabricated filters comprising two or more tapered pores. The substrate on which the filter pores, slots or openings are fabricated or machined may be silicon, silicon dioxide, plastic, glass, ceramics or other solid materials. The solid materials may be porous or non-porous. Those who are skilled in microfabrication and micromachining fabrication may readily choose and determine the fabrication protocols and materials to be used for fabrication of particular filter geometries.

Using the microfabrication or micromachining methods, the filter slots, pores or openings can be made with precise geometries. Depending on the fabrication methods or materials used, the accuracy of a single dimension of the filter slots (e.g. slot length, slot width) can be within 20%, or less than 10%, or less than 5%. Thus, the accuracy of the critical, single dimension of the filter pores (e.g. slot width for oblong or quadrilateral shaped slots) for the filters of the present invention are made within, preferably, less than 2 microns, more preferably, less than 1 micron, or even more preferably less than 0.5 micron.

Preferably, filters of the present invention can be made using the track-etch technique, in which filters made of glass, silicon, silicon dioxides, or polymers such as polycarbonate or polyester with discrete pores having relatively-uniform pore sizes are made. For example, the filter can be made by adapting and applying the track-etch technique described for Nucleopore Track-etch membranes to filter substrates. In the technique used to make membrane filters, a thin polymer film is tracked with energetic heavy ions to produce latent tracks on the film. The film is then put in an etchant to produce pores.

Preferred filters for the cell separation methods and systems of the present invention include microfabricated or micromachined filters that can be made with precise geometries for the openings on the filters. Individual openings are isolated with similar or almost identical feature sizes and are patterned on a filter. The openings can be of different shapes such as, for example, circular, quadrilateral, or elliptical. Such filters allow precise separation of particles based on their sizes and other properties.

In a preferred embodiment of a microfabricated filter, individual pores are isolated and of a cylindrical shape, and the pore size is within a 20% variation, where the pore size is calculated by the smallest and largest dimension of the pore (width and length, respectively).

### II. Method of Separating a Target Component of a Fluid Sample Using Microfiltration

In another aspect, the present invention provides methods of separating a target component of a fluid sample using filtration through a filtration chamber of the present invention that comprises a microfabricated filter enclosed in a housing. The filtration chamber may be configured to allow substantially anti-parallel flow in the antechamber and post-filtration subchamber. The surface of the filter and/or the inner surface of the housing may be modified by vapor deposition, sublimation, vapor-phase surface reaction, or particle sputtering to produce a uniform coating. In some embodiments, the surface of the filter and/or the inner surface of said housing are modified by vapor deposition, sublimation, vapor-phase surface reaction, or particle sputtering to produce a uniform coating. The method includes: dispensing a sample into a filtration chamber that comprises or engages a microfabricated filter enclosed in a housing; providing fluid flow of the sample through the filtration chamber, such that the target component of the fluid sample flows through or is retained by the one or more microfabricated filters. Separation of the components may be based on the size, shape, deformability, binding affinity and/or binding specificity of the components.

In some embodiments, the method may further comprise manipulating the fluid sample with a physical force, wherein said manipulation is effected through a structure that is external to the filter and/or a structure that is built-in on the filter. In some embodiments, the method may further comprise collecting the target component, such as nucleated cells or rare cells from said filtration chamber. In some embodiments, filtration can separate soluble and small components of a sample from at least a portion of the nucleated cells or rare cells that are in the sample, in order to concentrate the cells to facilitate further separation and analysis. In some aspects, filtration can remove undesirable components from a sample, such as, but not limited to, undesirable cell types. Where filtration reduces the volume of a sample by at least 50% or removes greater than 50% of the cellular components of a sample, filtration can be considered a debulking step. The present invention contemplates the use of filtration for debulking as well as other functions in the processing of a fluid sample, such as, for example, concentration of sample components or separation of sample components (including, for example, removal of undesirable sample components and retention of desirable sample components).

Fluid sample preparation and rare cell enrichment methods known in the art and disclosed U.S. patent application Ser. No. 11/777,962, filed Jul. 13, 2007, U.S. patent application Ser. No. 11/497,919, filed Aug. 2, 2006, U.S. patent application Ser. No. 11/264,413, filed Sep. 15, 2004, U.S. patent application Ser. No. 10/701,684, filed Nov. 4, 2003, U.S. patent application Ser. No. 10/268,312, filed Oct. 10, 2002, hereby incorporated by reference for all disclosure of blood sample preparation and rare cell isolation from blood samples, can be combined with the methods and designs disclosed herein.

Sample

A sample can be any fluid sample, such as an environmental sample, including air samples, water samples, food samples, and biological samples, including suspensions, extracts, or leachates of environmental or biological samples. Biological samples can be blood, a bone marrow sample, an effusion of any type, ascitic fluid, pelvic wash fluid, or pleural fluid, spinal fluid, lymph, serum, mucus, sputum, saliva, urine, semen, ocular fluid, extracts of nasal, throat or genital swabs, cell suspension from digested tissue, or extracts of fecal material. Biological samples can also be samples of organs or tissues, including tumors, such as fine needle aspirates or samples from perfusions of organs or tissues. Biological samples can also be samples of cell cultures, including both primary cultures and cell lines. The volume of a sample can be very small, such as in the microliter range, and may even require dilution, or a sample can be very large, such as up to about two liters for ascites fluid. A preferred sample is a blood sample.

A blood sample can be any blood sample, recently taken from a subject, taken from storage, or removed from a source external to a subject, such as clothing, upholstery, tools, etc. A blood sample can therefore be an extract obtained, for example, by soaking an article containing blood in a buffer or solution. A blood sample can be unprocessed or partially processed, for example, a blood sample that has been dialyzed, had reagents added to it, etc. A blood sample can be of any volume. For example, a blood sample can be less than five microliters, or more than 5 liters, depending on the application. Preferably, however, a blood sample that is processed using the methods of the present invention will be from about 10 microliters to about 2 liters in volume, more preferably from about one milliliter to about 250 milliliters in volume, and most preferably between about 5 and 50 milliliters in volume.

The rare cells to be enriched from a sample can be of any cell type present at less than one million cells per milliliter of fluid sample or that constitute less than 1% of the total nucleated cell population in a fluid sample. Rare cells can be, for example, bacterial cells, fungal cells, parasite cells, cells infected by parasites, bacteria, or viruses, or eukaryotic cells such as but not limited to fibroblasts or blood cells. Rare blood cells can be RBCs (for example, if the sample is an extract or leachate containing less than one million red blood cells per milliliter), subpopulations of blood cells and blood cell types, such as WBCs, or subtypes of WBCs (for example, T cells or macrophages), nucleated red blood cells, or can be fetal cells (including but not limited to nucleated red blood cells, trophoblasts, granulocytes, or monocytes). Rare cells can be stem or progenitor cells of any type. Rare cells can also be cancer cells, including but not limited to neoplastic cells, malignant cells, and metastatic cells. Rare cells of a blood sample can also be non-hematopoietic cells, such as but not limited to epithelial cells.

Maternal Blood Sample Selection for Fetal Cell Isolation

The present invention includes methods for rare cell isolation from blood samples that include the selection of a blood sample of a particular gestational age for isolation of particular fetal cell types.

In one preferred embodiment of the present invention, a maternal blood sample for the isolation of fetal nucleated cells is selected to be from the gestational age of between about 4 weeks and about 37 weeks, preferably about 7 weeks and about 24 weeks, and more preferably between about 10 weeks and about 20 weeks. In this embodiment, a maternal blood sample for the isolation of fetal nucleated cells is drawn from a pregnant subject at the gestational age of between about 4 weeks and about 37 weeks, preferably about 7 weeks and about 24 weeks, and more preferably between about 10 weeks and about 20 weeks. As used herein, a pregnant subject can also include a woman of the given gestational age that has aborted within twenty-four hours of the blood sample draw.

Use of the Second Wash Supernatant for Isolation Fetal Cells from a Maternal Blood Sample

The present invention also includes methods for isolating fetal cells from a maternal blood sample in which the supernatant of a second centrifugation performed on the blood sample to wash the cells prior to a debulking or separation step is used as at least a part of the sample from which fetal cells are isolated.

Dispensing of Sample into Filtration Chamber

A sample can be dispensed into a filtration chamber of the present invention by any convenient means. As non-limiting examples, sample can be introduced using a conduit (such as tubing) through which a sample is pumped or injected into the chamber, or can be directly poured, injected, or dispensed or pipetted manually, by gravity feed, or by a machine. Dispensing of a sample into a filtration chamber of the present invention can be directly into the filtration chamber, via a loading reservoir that feeds directly or indirectly into a filtration chamber, or can be into a conduit that leads to a filtration chamber, or into a vessel that leads, via one or more conduits, to a filtration chamber. A needle (or any fluid drawing device) in fluid communication with tubing or a chamber can also be used to enter a tube. The needle may collect cells from a tube containing a solution and dispense the solution into another chamber using a device to push or pull a solution (e.g. pump or syringe).

Filtering

Following the addition to a filtration chamber of the present invention, filtering is effected by providing fluid flow through the chamber. Fluid flow can be provided by any means, including positive or negative pressure (for example, by a manual or machine operated syringe-type system), pumping, or even gravity. The filtration chamber can have ports that are connected to conduits through which a buffer or solution and the fluid sample or components thereof can flow. A filtration unit can also have valves that can control fluid flow through the chamber. When the sample is added to the filtration chamber, and fluid flow is directed through the chamber, filter slots can allow the passage of fluid, soluble components of the samples, and filterable non-soluble components of a fluid sample through a filter, but, because of the slot dimensions, can prevent the passage of other components of the fluid sample through the filter.

In some embodiments, fluid flow in the antechamber and post-filtration subchamber are substantially anti-parallel. The flow can be effected by automated means through the inflow and/or outflow ports of the filtration chamber. In embodiments wherein an additional inflow port is provided, a fluid flow of a solution substantially perpendicular to the anti-parallel flow may be introduced. For example, wherein a suprafilter is included that divides the antechamber into a suprachamber and an antechamber, the antechamber may be used for a fluid flow across the filter(s) to push the components of the fluid sample across the filter(s).

Preferably, fluid flow through a filtration chamber of the present invention is automated, and performed by a pump or positive or negative pressure system, but this is not a requirement of the present invention. The optimal flow rate will depend on the sample being filtered, including the concentration of filterable and non-filterable components in the sample and their ability to aggregate and clog the filter. For example, the flow rate through the filtration chamber can be from less than 1 milliliter per hour to more than 1000 milliliters per hour, and flow rate is in no way limiting for the practice of the present invention. Preferably, however, filtration of a blood sample occurs at a rate of from 5 to 500 milliliters per hour, and more preferably at a rate of between about 5 and about 40 milliliters per hour.

Blood (either whole blood or diluted whole blood) may be introduced into the antechamber by engaging the delivery mechanism, namely a pipette sealed to the inflow port and driven by a pump or gravity, or by any flow generating method, and delivering a known quantity of the blood continuously through the antechamber of the filter and collecting the debulked blood from the outflow port of the antechamber. Alternatively, a fixed volume of blood or blood mixture may be delivered into a reservoir that is part of the inflow port, and a flow mechanism will engage with the outflow port of the antechamber and draw said sample continuously through the antechamber until the desired volume is collected.

During the passage of the blood through the top chamber, the bottom chamber will have an inflow and an outflow port, both of which will be connected to pumps where the outflow rate will be greater than the inflow rate such that some contents from the top chamber are slowly drawn across the filter and into the post-filtration subchamber. The flow through the post-filtration subchamber will preferably be in the opposite direction to flow in the top chamber, or antiparallel flow, such that particles traversing the filter will not have an opportunity to diffuse back through the filter into a region of the blood which may not contain as many of those particles, as depicted in FIG. 33. In so doing, the blood will be cleared of the smaller particles, namely platelets and/or red blood cells, and preferably both.

The traversing of the filter material may optionally be aided by electrostatic, electromagnetic, electrophoretic, or electroosmotic flow by introducing two or more electrodes into any of the ports, or by connecting to electrodes integrated into the unit, potentially forming the ceiling and floor of the opposing chambers. Optionally, the separation of the particles by size may be aided by oscillatory flow produced by oscillating the pumps or by introducing an acoustic force to the flow across the filters. This acoustic force may be a pressure wave from impact anywhere along the fluidics, or created by a speaker or piezoelectric device embedded in the waste chamber (post-filtration subchamber) or anywhere along the post-filtration subchamber fluidics.

In some embodiments, the device may be operated oriented upside-down, or on its side such that the function of the bottom chamber of removing unwanted particles may actually be on a side chamber or top chamber.

In fabricating the filter slots through the filter substrate, slight tapering of the slot along the slot depth direction can occur. Thus a particular slot width may not be maintained constant throughout the entire depth of the filter and the slot width on one surface of the filter is typically larger than the width on the opposite surface. In utilizing such filters with tapered slot width, it is preferred to have the narrow-slot side of the filter facing the sample, so that during filtering the sample goes through the narrow-width side of the slot first and then filtered cells exit at the wide-width side of the slot. This avoids trapping cells that are being filtered within the funnel-shaped slots. However, the orientation of a filter with one or more tapered slots is not a restriction in using the filters of the present invention. Depending on specific applications, the filters can also be used in the orientation such that the wide-width side of the filter slots faces the sample.

In the methods of the present invention, preferably desirable components, such as rare cells whose enrichment is desired, are retained by the filter. Preferably, in the methods of the present invention as rare cells of interest of the sample are retained by the filter and one or more undesirable components of the sample flow through the filter, thereby enriching the rare cells of interest of the sample by increasing the proportion of the rare cells to total cells in the filter-retained portion of the sample, although that is not a requirement of the present invention. For example, in some embodiments of the present invention, filtration can enrich rare cells of a fluid sample by reducing the volume of the sample and thereby concentrating rare cells.

Specific Binding Member for Removing Undesirable Components

In addition to the components of a sedimenting solution of the present invention, a combined solution of the present invention can comprise at least one specific binding member that can selectively bind undesirable components of a blood sample (such as but not limited to white blood cells, platelets, serum proteins) and have less binding to desirable components. One or more specific binding members that can selectively bind non-RBC undesirable components of a blood sample can be used to remove the undesirable components of the sample, increasing the relative proportion of rare cells in the sample, and thus contribute to the enrichment of rare cells of the sample. By “selectively binds” is meant that a specific binding member used in the methods of the present invention to remove one or more undesirable sample components does not appreciably bind to rare cells of interest of the fluid sample. By “does not appreciably bind” is meant that not more than 30%, preferably not more than 20%, more preferably not more than 10%, and yet more preferably not more than 1.0% of one or more rare cells of interest are bound by the specific binding member used to remove non-RBC undesirable components from the fluid sample. In many cases, the undesirable components of a blood sample will be white blood cells. In preferred embodiments of the present invention, a combined solution of the present invention can be used for sedimenting red blood cells and selectively removing white blood cells from a blood sample.

A specific binding member that can specifically bind white blood cells can be as non-limiting examples, an antibody, a ligand for a receptor, transporter, channel or other moiety of the surface of a white blood cell, or a lectin or other protein that can specifically bind particular carbohydrate moieties on the surface of a white blood cell (for example, a selectin).

Preferably, a specific binding member that selectively binds white blood cells is an antibody that binds white blood cells but does not appreciably bind fetal nucleated cells, such as, for example, an antibody to CD3, CD11b, CD14, CD17, CD31, CD45, CD50, CD53, CD63, CD69, CD81, CD84, CD102, or CD166. Antibodies can be purchased commercially from suppliers such as, for example Dako, BD Pharmingen, Antigenix America, Neomarkers, Leinco Technologies, Research & Diagnostic Systems, Serotec, United States Biological, Bender Medsystems Diagnostics, Ancell, Leinco Technologies, Cortex Biochem, CalTag, Biodesign, Biomeda, Accurate Chemicals & Scientific and Chemicon International. Antibodies can be tested for their ability to bind an efficiently remove white blood cells and allow for the enrichment of rare cells of interest from a sample using capture assays well known in the art.

Specific binding members that selectively bind to one or more undesirable components of the present invention can be used to capture one or more non-RBC undesirable components, such that one or more desirable components of the fluid sample can be removed from the area or vessel where the undesirable components are bound. In this way, the undesirable components can be separated from other components of the sample that include the rare cells to be separated. The capture can be affected by attaching the specific binding members that recognize the undesirable component or components to a solid support, or by binding secondary specific binding members that recognize the specific binding members that bind the undesirable component or components, to a solid support, such that the undesirable components become attached to the solid support. In preferred embodiments of the present invention, specific binding members that selectively bind undesirable sample components provided in a combined solution of the present invention are coupled to a solid support, such as microparticles, but this is not a requirement of the present invention.

Magnetic beads are preferred solid supports for use in the methods of the present invention to which specific binding members that selectively bind undesirable sample components can be coupled. Magnetic beads are known in the art, and are available commercially. Methods of coupling molecules, including proteins such as antibodies and lectins, to microparticles such as magnetic beads are known in the art. Preferred magnetic beads of the present invention are from 0.02 to 20 microns in diameter, preferably from 0.05 to 10 microns in diameter, and more preferably from 0.05 to 5 microns in diameter, and even more preferably from 0.05 to 3 microns in diameter and are preferably provided in a combined solution of the present invention coated with a primary specific binding member, such as an antibody that can bind a cell that is to be removed from the sample, or a secondary specific binding member, such as streptavidin, that can bind primary specific binding members that bind undesirable sample components (such as biotinylated primary specific binding members).

In preferred embodiments of the present invention, the fluid sample is a maternal blood sample, the rare cells whose separation is desirable are fetal cells, and the undesirable components of the sample to be removed from the sample are white blood cells. In these embodiments, a specific binding member that selectively binds white blood cells is used to remove the white blood cells from the sample by magnetic capture. Preferably, the specific binding member provided is attached to magnetic beads for direct capture, or, is provided in biotinylated form for indirect capture of white blood cells by streptavidin-coated magnetic beads.

A combined solution for enriching rare cells of a blood sample of the present invention can also include other components, such as, but not limited to, salts, buffering agents, agents for maintaining a particular osmolality, chelators, proteins, lipids, small molecules, anticoagulants, etc. For example, in some preferred aspects of the present invention, a combined solution comprises physiological salt solutions, such as PBS, PBS lacking calcium and magnesium or Hank's balanced salt solution. In some preferred aspects of the present invention, EDTA or heparin are present to prevent red blood cell clotting.

The present invention also includes the use of an antibody or molecule capable of specifically binding a platelet or a molecule associated with a platelet. As a non-limiting example, antibodies or molecules or the present invention may specifically bind CD31, CD36, CD41, CD42(a,b,c), CD51 or CD51/61. CD31 is an endothelial and platelet cell marker that has minimal binding to fetal cells. Its use in separating platelets from a blood sample is described in the examples.

Improved Magnet Configurations for Capture of Sample Components

A debulked sample, such as a debulked blood sample, can be incubated with one or more specific binding members, such as, but not limited to, antibodies, that specifically recognize one or more undesirable components of a fluid sample. Where a filtration chamber has been used for debulking the sample, mixing and incubation of one or more specific binding members with the sample can optionally be performed in a filtration chamber. The one or more undesirable components can be captured, either directly or indirectly, via their binding to the specific binding member. For example, a specific binding member can be bound to a solid support, such as a bead, membrane, or column matrix, and following incubation of the fluid sample with the specific binding member, the fluid sample, containing unbound components, can be removed from the solid support. Alternatively, one or more primary specific binding members can be incubated with the fluid sample, and, preferably following washing to remove unbound specific binding members, the fluid sample can be contacted with a secondary specific binding member that can bind or is bound to a solid support. In this way the one or more undesirable components of the sample can become bound to a solid support, enabling separation of the undesirable components from the fluid sample.

In a preferred aspect of the present invention, a debulked blood sample from a pregnant individual is incubated with magnetic beads that are coated with antibody that specifically binds white blood cells and does not appreciably bind fetal nucleated cells. The magnetic beads are collected using capture by activated electromagnetic units (such as on an electromagnetic chip), or capture by at least one permanent magnet that is in physical proximity to a vessel, such as a tube or column, that contains the fluid sample. After capture of the magnetic beads by the magnet, the remaining fluid sample is removed from the vessel. The sample can be removed manually, such as by pipetting, or by physical forces such as gravity, or by fluid flow through a separation column. In this way, undesirable white blood cells can be selectively removed from a maternal blood sample. The sample can optionally be further filtered using a microfabricated filter of the present invention. Filtration preferably removes residual red blood cells from the sample and can also further concentrate the sample.

In one preferred embodiment, after incubation of magnetic beads that comprise a specific binding member that specifically bind undesirable components with a sample, the sample is transported through a separation column that comprises or engages at least one magnet. As the sample flows through the column, undesirable components that are bound to the magnetic beads adhere to one or more walls of the tube adjacent to the magnet or magnets. An alternative embodiment uses a magnetic separator, such as the magnetic separator manufactured by Immunicon (Huntingdon Valley, Pa.). Magnetic capture can also employ electromagnetic chips that comprise electromagnetic physical force-generating elements, such as those described in U.S. Pat. No. 6,355,491 entitled “Individually Addressable Micro-Electromagnetic Unit Array Chips” issued Mar. 12, 2002 to Zhou et al., U.S. application Ser. No. 09/955,343 having attorney docket number ART-00104.P.2, filed Sep. 18, 2001, entitled “Individually Addressable Micro-Electromagnetic Unit Array Chips” and U.S. application Ser. No. 09/685,410 having attorney docket number ART-00104.P.1.1, filed Oct. 10, 2000, entitled “Individually Addressable Micro-Electromagnetic Unit Array Chips in Horizontal Configurations”. In yet another preferred embodiment, a tube that contains the sample and magnetic beads is positioned next to one or more magnets for the capture of non-desirable components bound to magnetic beads. The supernatant, depleted of the one or more non-desirable components, can be removed from the tube after the beads have collected at the tube wall.

In some preferred embodiments of the present invention, removal of white blood cells from a sample is performed simultaneously with debulking the blood sample by selective sedimentation of red blood cells. In these embodiments, a solution that selectively sediments red blood cells is added to a blood sample, and a specific binding member that specifically binds white blood cells that is bound to a solid support, such as magnetic beads, is added to the blood sample. After mixing, red blood cells are allowed to settle, and white blood cells are captured, such as by magnetic capture. This can be conveniently performed in a tube to which a sedimenting solution and the specific binding member, preferably bound to magnetic beads, can be added. The tube can be rocked for a period of time for mixing the sample, and then positioned next to one or more magnets for the capture of the magnetic beads. In this way, in a single incubation and separation step, approximately 99% of RBCs and 99% of WBCs can be removed from a sample. The supernatant can be removed from the tube and subjected to filtration using a microfabricated filter of the present invention. Filtration removes remaining RBCs, resulting in a sample in which rare cells, such as, for example, fetal cells, cancer cells, or stem cells, have been enriched.

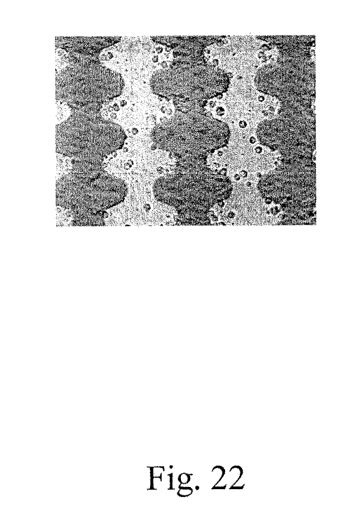

Undesirable components of a sample can be removed by methods other than those using specific binding members. For example, the dielectrical properties of particular cell types can be exploited to separate undesirable components dielectrophoretically. For example, FIG. 22 depicts white blood cells of a diluted blood sample retained on electrodes of a dielectrophoresis chip after red blood cells have been washed through the chamber.

Combined Solution for Sedimenting Red Blood Cells and Selectively Removing Undesirable Sample Components of a Blood Sample

In preferred embodiments of the present invention, a solution that sediments red blood cells can also include one or more additional specific binding members that can be used to selectively remove undesirable sample components other than red blood cells from the blood sample. In this regard, the present invention includes a combined sedimenting solution for enriching rare cells of a blood sample that sediments red blood cells and provides reagents for the removal of other undesirable components of the sample. Thus a combined solution for processing a blood sample comprises: dextran; at least one specific binding member that can induce agglutination of red blood cells; and at least one additional specific binding member that can specifically bind undesirable components of the sample other than RBCs.

Additional Enrichment Steps

The present invention also contemplates using filtration in combination with other steps that can be used in enriching rare cells of a fluid sample. For example, debulking steps or separation steps can be used prior to or following filtration, such as but not limited to as disclosed in U.S. patent application Ser. No. 10/701,684, entitled “Methods, Compositions, and Automated Systems for Separating Rare Cells from Fluid Samples” filed Nov. 4, 2003, U.S. patent application Ser. No. 10/268,312, entitled “Methods, Compositions, and Automated Systems for Separating Rare Cells from Fluid Samples” filed Oct. 10, 2002, both of which are incorporated herein by reference for all disclosure relating to debulking and separation procedures that can be used in enriching rare cells of a fluid sample.

### III. Methods of Using Automated Filtration Unit for Separating a Target Component of a Fluid Sample

In yet another aspect, the present invention also includes method of separating a target component in a fluid sample using the automated filtration unit disclosed herein, comprising: a) dispensing the fluid sample into the filtration chamber; and b) providing a fluid flow of the fluid sample through the antechamber of the filtration chamber and a fluid flow of a solution through the post-filtration subchamber of the filtration chamber, wherein the target component of the fluid sample is retained by or flows through the filter.

Sample

A sample can be any fluid sample, such as an environmental sample, including air samples, water samples, food samples, and biological samples, including extracts of biological samples. Biological samples can be blood, a bone marrow sample, an effusion of any type, ascitic fluid, pelvic wash fluid, pleural fluid, spinal fluid, lymph, serum, mucus, sputum, saliva, urine, vaginal or uterine washes, semen, ocular fluid, extracts of nasal, throat or genital swabs, cell suspension from digested tissue, or extracts of fecal material. Biological samples can also be samples of organs or tissues, including tumors, such as fine needle aspirates or samples from perfusions of organs or tissues. Biological samples can also be samples of cell cultures, including both primary cultures and cell lines. The volume of a sample can be very small, such as in the microliter range, and may even require dilution, or a sample can be very large, such as up to 10 liters for ascites fluid. One preferred sample is a urine sample. Another preferred sample is a blood sample. Also contemplated is a sample of lab-cultured cells of either mixed types or mixed sizes or cells that contain contaminants or unbound reactants that must be removed from the sample. In some embodiments, the fluid sample is a prepared cell sample with labeling reagent meant to bind or absorb or be taken up by the cells and the component being removed is the unbound or interstitial components of the labeling reagent.

A biological sample can be any sample, recently taken from a subject, taken from storage, or removed from a source external to a subject, such as clothing, upholstery, tools, etc. As an example, a blood sample can therefore be an extract obtained, for example, by soaking an article containing blood in a buffer or solution. A biological sample can be unprocessed or partially processed, for example, a blood sample that has been dialyzed, had reagents added to it, etc. A biological sample can be of any volume. For example, a blood sample can be less than five microliters, or more than 5 liters, depending on the application. Preferably, however, a biological sample that is processed using the methods of the present invention will be from about 10 microliters to about 2 liters in volume, more preferably from about one milliliter to about 250 milliliters in volume, and most preferably between about 5 and 50 milliliters in volume.

Introduction of Sample

In some preferred embodiments of the present invention, one or more samples can be provided in one or more tubes that can be placed in a rack of the automated system. The rack can be automatically or manually engaged with the automated system for sample manipulations.

Alternatively, a sample can be dispensed into an automated system of the present invention by pipetting or injecting the sample through an inlet of an automated system, or can be poured, pipetted, or pumped into a conduit or reservoir of the automated system. In most cases, the sample will be in a tube that provides for optimal separation of sedimented cells, but it can be in any type of vessel for holding a liquid sample, such as a plate, dish, well, or chamber.

Prior to the dispensing of a sample into a vessel or chamber of the automated system, solutions or reagents can optionally be added to the sample. Solutions or reagents can optionally be added to a sample before the sample is introduced into an automated system of the present invention, or after the sample is introduced into an automated system of the present invention. If a solution or reagent is added to a sample after the sample is introduced into an automated system of the present invention, it can optionally be added to the sample while the sample is contained within a tube, vessel, or reservoir prior to its mixing or incubation step, the settling step, or its introduction into a filtration chamber. Alternatively, a solution or reagent can be added to a sample through one or more conduits, such as tubing, where the mixing of sample with a solution or reagent takes place in conduits. It is also possible to add one or more solutions or reagents after the sample is introduced into a chamber of the present invention (such as, but not limited to, a filtration chamber), by adding one or more of these directly to the chamber, or through conduits that lead to the chamber.

The sample (and, optionally, any solutions, or reagents) can be introduced into the automated system by positive or negative pressure, such as by a syringe-type pump. The sample can be added to the automated system all at once, or can be added gradually, so that as a portion of the sample is being filtered, additional sample is added. A sample can also be added in batches, such that a first portion of a sample is added and filtered through a chamber, and then further batches of a sample are added and filtered in succession.

Filtering the Sample Through a Filtration Chamber of the Automated Filtration Unit

A sample can be filtered in an automated filtration unit of the present invention before or after undergoing one or more debulking steps or one or more separation steps. These debulking or separation steps can include but are not limited to a RBC sedimentation step or removal by specific binding members. The sample can be directly transferred to a filtration chamber (such as by manual or automated dispensing) or can enter a filtration chamber through a conduit. After a sample is added to a filtration chamber, it is filtered to reduce the volume of the sample, and, optionally, to remove undesirable components of a sample. To filter the sample, fluid flow is directed through the chamber. Fluid flow through the chamber is preferably directed by automatic rather than manual means, such as by an automatic syringe-type pump. The pump can operate by exerting positive or negative pressure through conduits leading to the filtration chamber.

In some embodiments, fluid flow in the antechamber and post-filtration subchamber are substantially anti-parallel. The flow can be effected by automated means through the inflow and/or outflow ports of the filtration chamber. In embodiments wherein an additional inflow port is provided, a fluid flow of a solution substantially perpendicular to the anti-parallel flow may be introduced. For example, wherein a suprafilter is included that divides the antechamber into a suprachamber and an antechamber, the antechamber may be used for a fluid flow across the filter(s) to push the components of the fluid sample across the filter(s).

The flow rate in the antechamber and the flow rate in the post-filtration subchamber may be different, such that a fluidic force is generated on components of the fluid sample to flow from the antechamber to the post-filtration subchamber. As used herein, “filter rate” refers to the fluidic flow rate across the filter; “feed rate” refers to the fluidic flow rate in the antechamber; and “buffer rate” and “waste rate” refers to the fluidic flow rate in inflow port and outflow port of the post-filtration subchamber, respectively. Further, the inflow rate and outflow rate of the post-filtration subchamber may be different to generate the desired fluidic force to direct the fluid flow across the filter. For example, when the outflow rate is greater than the inflow rate in the post-filtration subchamber, a fluidic force is generated from the antechamber to the post-filtration subchamber, so that components of the fluidic sample in the antechamber are drawn to the post-filtration subchamber through the filter.

The rate of fluid flow through a filtration chamber can be any rate that allows for effective filtering, and for a whole blood sample is preferably up to about 10 mL/min, more preferably between about 10 and about 500 μL/min, and most preferably between about 80 and about 140 μL/min. The rate of fluid flow in the antechamber may be about 1-10 times the filter rate. Following the addition of a sample to a filtration chamber, a pump or fluid dispensing system can optionally direct fluid flow of a buffer or solution into the chamber to wash additional filterable sample components through the chamber.

When the sample is added to the filtration chamber, and fluid flow is directed through the chamber, pores or slots in the filter or filters can allow the passage of fluid, soluble components of the samples, and some non-soluble components of a fluid sample through one or more filters, but, because of their dimensions, can prevent the passage of other components of the fluid sample through the one or more filters.

For example, in preferred embodiments a fluid sample can be dispensed into a filtration chamber that comprises at least one filter that comprises a plurality of slots. The chamber can have ports that are optionally connected to conduits through which a buffer or solution and the fluid sample or components thereof can flow. When the sample is added to the chamber, and fluid flow is directed through the chamber, the slots can allow the passage of fluid and, optionally, some components of a fluid sample through the filter, but prevent the passage of other components of the fluid sample through the filter.

In some embodiments of the present invention, an active chip that is part of the filtration chamber can be used to mix the sample during the filtration procedure. For example, an active chip can be an acoustic chip that comprises one or more acoustic elements. When an electric signal from a power supply activates the acoustic elements, they provide vibrational energy that causes mixing of the components of a sample. An active chip that is part of a filtration chamber of the present invention can also be a dielectrophoresis chip that comprises microelectrodes on the surface of a filter. When an electric signal from a power supply is transmitted to the electrodes, they provide a negative dielectrophoretic force that can repel components of a sample from the filter surface. In this embodiment, the electrodes on the surface of the filter/chip are preferably activated intermittently, when fluid flow is halted or greatly reduced.

Mixing of a sample during filtration is performed to avoid reductions in the efficiency of filtration based on aggregation of sample components, and in particular their tendency to collect, in response to fluid flow through the chamber, at positions in the chamber where filtering based on size or shape occurs, such as dams, slots, etc. Mixing can be done continuously through the filtration procedure, such as through a continuous activation of acoustic elements, or can be done in intervals, such as through brief activation of acoustic elements or electrodes during the filtration procedure. Where dielectrophoresis is used to mix a sample in a filtration chamber, preferably the dielectrophoretic force is generated in short intervals (for example, from about two seconds to about 15 minutes, preferably from about two to about 30 seconds in length) during the filtration procedure; for example, pulses can be given every five seconds to about every fifteen minutes during the filtration procedure, or more preferably between about every ten seconds to about every one minute during the filtration procedure. The dielectrophoretic forces generated serve to move sample components away from features that provide the filtering function, such as, but not limited to, slots.

During the filtration procedure, filtered sample fluid can be removed from the filtration chamber by automated fluid flow through conduits that lead to one or more vessels for containing the filtered sample. In preferred embodiments, these vessels are waste receptacles. After filtration, fluid flow can optionally be directed in the reverse direction through the filter to suspend retained components that may have settled or lodged against the filter.

After the filtration procedure (and optionally, a mixing and incubation with one or more specific binding members), sample components that remain in the filtration chamber after the filtration procedure can be directed out of the chamber through additional ports and conduits that can lead to collection tubes or vessels or to other elements of the automated system for further processing steps, or can be removed from the filtration chamber or a collection vessel by pipetting or a fluid uptake means. Ports can have valves or other mechanisms for controlling fluid flow. The opening and closing of ports can be automatically controlled. Thus, ports that can allow the flow of debulked (retained) sample out of a filtration chamber (such as to other chambers or collection vessels) can be closed during the filtration procedure, and conduits that allow the flow of filtered sample out of a filtration chamber can optionally be closed after the filtration procedure to allow efficient removal of remaining sample components.

Rinsing

After filtering of the fluidic sample, optionally buffer can be washed through the filtration chamber to wash through any residual components, such as undesirable cells. The buffer can be conveniently directed through the filtration chamber in the same manner as the sample, that is, preferably by automated fluid flow such as by a pump or pressure system, or by gravity, or the buffer can use a different fluid flow means than the sample. One or more washes can be performed, using the same or different wash buffers. In addition, optionally air can be forced through the filtration chamber, for example by positive pressure or pumping, to push residual cells through the filtration chamber. Also, it is possible to have one or more washes back flushed into the filtration chamber to assist in the washing of the chamber or removal of undesirable cells or assist in the recovery of desirable cells.

During the rinsing step, the feed rate may be less than or equal to the filter rate, such as the rinsing reagent, such as EDTA, may cross the filter into the antechamber, removing any residual component blocking the slots on the filter.

Labeling

Optionally, the separated target component may be labeled using the automated filtration unit of the present invention. For example, separated nucleated cells or rare cells may be labeled with an antibody or assay reagent for further analysis. In some embodiments, the antibody or assay reagent may be conjugated with a detectable molecule, such as a radioactive or fluorescent dye.

The labeling reagent may be added to the collection chamber, where the target component is collected after filtration. Alternatively, the labeling reagent may be added to the antechamber or the post-filtration subchamber, depending on where the target component is located. Adding the reagent may be carried out by the fluidic pumps and conduits of the automated filtration unit, and controlled by the control algorithm.

During the labeling step the fluid flow may be paused in the filtration chamber to allow effective binding between the target component and the labeling reagent. A labeling time of suitable length may be used, for example, about 1-10 min.

After the labeling step, the unbound labeling reagent may be rinsed away by adding a rinsing buffer to the filtration chamber.

Recovering

During the recovering step, separated target component is collected. In some embodiments, the target component on the filter is lifted from filter slots and pushed into the collection chamber. A fluidic force may be generated that lift any components blocking the filter slots, for example, by pausing the outflow in the post-filtration subchamber, or by reducing the outflow rate of the post-filtration subchamber so that it is less than the inflow rate of the post-filtration subchamber. Alternatively, the lifting step may be via increasing the buffer rate and the feed rate to about 1-10 mL/min and about 0.5-5 mL/min, respectively. The duration of the lifting step may vary, for example, from 10 ms to 1 s or longer. Further, the lifting step may be performed intermittently throughout the filtration, so that optimal filtration effect is achieved. In some embodiments, the speed at which the wash buffer flows through the chamber may be greater than that of a sample.

Selective Removal of Undesirable Components of a Sample

Optionally, sample components that remain in the filtration chamber either before, during, or after the filtration procedure can be directed by fluid flow to an element of the automated system in which undesirable components of a sample can be separated from the sample. In some embodiments of the present invention, prior to either adding the sample to the filtration chamber or removing the debulked sample retained in the filtration chamber, one or more specific binding members can be added to the debulked sample and either mixed before the and afterwards in the filtration chamber, using, for example, one or more active chips that engage or are a part of the filtration chamber to provide physical forces for mixing. Preferably, one or more specific binding member is added to the debulked sample in the filtration chamber, ports of the chamber are closed, and acoustic elements are activated either continuously or in pulsed, during the incubation of debulked sample and specific binding members. Preferably, one or more specific binding members are antibodies that are bound to magnetic beads. The specific binding members can be antibodies that bind desirable sample components, such as fetal nucleated cells, but preferably the specific binding members are antibodies that bind undesirable sample components, such as white blood cells while having minimal binding to desirable sample components.

In preferred embodiments of the present invention, sample components that remain in the filtration chamber after the filtration procedure are incubated with magnetic beads, and following incubation, are directed by fluid flow to a separation column. Preferably, a separation column used in the methods of the present invention is a cylindrical glass, plastic, or polymeric column with a volumetric capacity of between about one milliliter and ten milliliters, having entry and exit ports at opposite ends of the column. Preferably, a separation column used in the methods of the present invention comprises or can be positioned alongside at least one magnet that runs along the length of the column. The magnet can be a permanent magnet, or can be one or more electromagnetic units on one or more chips that is activated by a power source.

Sample components that remain in the filtration chamber after the filtration procedure can be directed by fluid flow to a separation column. Reagents, preferably including a preparation of magnetic beads, can be added to the sample components before or after they are added to the chamber. Preferably, reagents are added prior to transfer of sample components to a separation chamber. Preferably a preparation of magnetic beads added to the sample comprises at least one specific binding member, preferably a specific binding member that can directly bind at least one undesirable component of the sample. However, it is also possible to add a preparation of magnetic beads that comprise at least one specific binding member that can indirectly bind at least one undesirable component of the sample. In this case, it is necessary to also add a primary specific binding partner that can directly bind undesirable components to the sample. A primary specific binding partner is preferably added to the sample before the preparation of magnetic beads comprising a secondary specific binding partner is added to the sample, but this is not a requirement of the present invention. Bead preparations and primary specific binding partners can be added to a sample before or after the addition of the sample to a separation column, separately or together.

In embodiments where magnetic beads comprise primary specific binding members, the sample and magnetic bead preparation are preferably incubated together for between about five and about sixty minutes before magnetic separation. In embodiments where a separation column comprises or is adjacent to one or more permanent magnets, the incubation can occur prior to the addition of the sample to the separation column, in conduits, chambers, or vessels of the automated system. In embodiments where a separation column comprises or is adjacent to one or more current-activated electromagnetic elements, the incubation can occur in a separation column, prior to activating the one or more electromagnetic elements. Preferably, however, incubation of a sample with magnetic beads comprising specific binding members occurs in a filtration chamber following filtration of the sample, and after conduits leading into and out of the filtration chamber has been closed.

Where magnetic beads comprising secondary specific binding members are employed, optionally more than one incubation can be performed (for example, a first incubation of sample with a primary specific binding member, and a second incubation of sample with beads comprising a secondary specific binding member). Separation of undesirable components of a sample can be accomplished by magnetic forces that cause the electromagnetic beads that directly or indirectly bind the undesirable components. This can occur when the sample and magnetic beads are added to the column, or, in embodiments where one or more electromagnetic units are employed, by activating the electromagnetic units with a power supply. Non-captured sample components can be removed from the separation column by fluid flow. Preferably, non-captured sample components exit the column through a portal that engages a conduit.

Separation of Desirable Components

After filtering, a sample can optionally be directed by fluid flow to a separation chamber for the separation of rare cells.

In preferred aspects in which undesirable components of a debulked sample have been removed in a separation column, the debulked sample is preferably but optionally transferred to a second filtration chamber prior to being transferred to a separation chamber for separation rare cells of the sample. A second filtration chamber allows for further reduction of the volume of a sample, and also optionally allows for the addition of specific binding members that can be used in the separation of rare cells and mixing of one or more specific binding members with a sample. Transfer of a sample from a separation column to a separation chamber is by fluid flow through conduits that lead from a separation column to a second filtration chamber. A second filtration chamber preferably comprises at least one filter that comprises slots, and fluid flow through the chamber at a rate of between about one and about 500 milliliters per hour, more preferably between about two and about 100 milliliters per hour, and most preferably between about five and about fifty milliliters per hour drives the filtration of sample. In this way, the volume of a debulked sample from which undesirable components have been selectively removed can be further reduced. A second filtration chamber can comprise or engage one or more active chips. Active chips, such as acoustic chips or dielectrophoresis chips, can be used for mixing of the sample prior to, during, or after the filtration procedure.

A second filtration chamber can also optionally be used for the addition of one or more reagents that can be used for the separation of rare cells to a sample. After filtration of the sample, conduits that carry sample or sample components out of the chamber can be closed, and one or more conduits leading into the chamber can be used for the addition of one or more reagents, buffers, or solutions, such as, but not limited to, specific binding members that can bind rare cells. The one or more reagents, buffers, or solutions can be mixed in the closed-off separation chamber, for example, by activation of one or more acoustic elements or a plurality of electrodes on one or more active chips that can produce physical forces that can move components of the sample and thus provide a mixing function. In preferred aspects of the present invention, magnetic beads that are coated with at least one antibody that recognizes a rare cell are added to the sample in the filtration chamber. The magnetic beads are added via a conduit, and are mixed with the sample by activation of one or more active chips that are integral to or engage a second filtration chamber. The incubation of specific binding members with a sample can be from about five minutes to about two hours, preferably from about eight to about thirty minutes, in duration, and mixing can occur periodically or continuously throughout the incubation.

It is within the scope of the present invention to have a second filtration chamber that is not used for the addition and mixing of one or more reagents, solutions, or buffers with a sample. It is also within the scope of the present invention to have a chamber that precedes a separation chamber for the separation of rare cells that can be used for the addition and mixing of one or more reagents, solutions, or buffers with a sample, but that does not perform a filtering function. It is also within the scope of the present invention to have a sample transferred from a separation column to a separation chamber, without an intervening filtration or mixing chamber. In aspects where the methods are directed toward the separation of rare cells from a blood sample, however, the use of a second filtration chamber that is also used for the addition and mixing of one or more reagents with a sample is preferred.

Sample is transferred to a separation chamber by fluid flow. Preferably, a separation chamber for the separation of rare cells comprises or engages at least one active chip that can perform a separation. Such chips comprise functional elements that can, at least in part, generate physical forces that can be used to move or manipulate sample components from one area of a chamber to another area of a chamber. Preferred functional elements of a chip for manipulating sample components are electrodes and electromagnetic units. The forces used to translocate sample components on an active chip of the present invention can be dielectrophoretic forces, electromagnetic forces, traveling wave dielectrophoretic forces, or traveling wave electromagnetic forces. An active chip used for separating rare cells is preferably part of a chamber. The chamber can be of any suitable material and of any size and dimensions, but preferably a chamber that comprises an active chip used for separating rare cells from a sample (a “separation chamber”) has a volumetric capacity of from about one microliter to ten milliliters, more preferably from about ten microliters to about one milliliter.

In some embodiments of the present inventions, the active chip is a dielectrophoresis or traveling wave dielectrophoresis chip that comprises electrodes. Such chips and their uses are described in U.S. application Ser. No. 09/973,629, entitled “An Integrated Biochip System for Sample Preparation and Analysis”, filed Oct. 9, 2001; U.S. application Ser. No. 09/686,737, filed Oct. 10, 2000 entitled “Compositions and Methods for Separation of Moieties on Chips”, U.S. application Ser. No. 09/636,104, filed Aug. 10, 2000, entitled “Methods for Manipulating Moieties in Microfluidic Systems”; and U.S. application Ser. No. 09/679,024 having attorney docket number 471842000400, entitled “Apparatuses Containing Multiple Active Force Generating Elements and Uses Thereof” filed Oct. 4, 2000; all incorporated by reference. Rare cells can be separated from a sample of the present invention by, for example, their selective retention on a dielectrophoresis chip, and fluid flow can remove non-retained components of the sample.

In other preferred embodiments of the present invention, the active chip is an electromagnetic chip that comprises electromagnetic units, such as, for example, the electromagnetic chips described in U.S. Pat. No. 6,355,491 entitled “Individually Addressable Micro-Electromagnetic Unit Array Chips” issued Mar. 12, 2002 to Zhou et al., U.S. application Ser. No. 09/955,343 having attorney docket number ART-00104.P.2, filed Sep. 18, 2001, entitled “Individually Addressable Micro-Electromagnetic Unit Array Chips”, and U.S. application Ser. No. 09/685,410 having attorney docket number ART-00104.P.1.1, filed Oct. 10, 2000, entitled “Individually Addressable Micro-Electromagnetic Unit Array Chips in Horizontal Configurations”. Electromagnetic chips can be used for separation by magnetophoresis or traveling wave electromagnetophoresis. In preferred embodiments, rare cells can be incubated, before or after addition to a chamber that comprises an electromagnetic chip, with magnetic beads comprising specific binding members that can directly or indirectly bind the rare cells. Preferably, in embodiments where rare cells are captured on an electromagnetic chip, the sample is mixed with the magnetic beads comprising a specific binding member in a mixing chamber. Preferably, a mixing chamber comprises an acoustic chip for the mixing of the sample and beads. The cells can be directed through conduits from the mixing chamber to the separating chamber. The rare cells can be separated from the fluid sample by magnetic capture on the surface of the active chip of the separation chamber, and other sample components can be washed away by fluid flow.

The methods of the present invention also include embodiments in which an active chip used for separation of rare cells is a multiple-force chip. For example, a multiple-force chip used for the separation of rare cells can comprise both electrodes and electromagnetic units. This can provide for the separation of more than one type of sample component. For example, magnetic capture can be used to isolated rare cells, while negative dielectrophoresis is used to remove undesirable cells from the chamber that comprises the multiple-force chip.

After the removal of undesirable sample components from the separation chamber, either through active physical forces such as negative dielectrophoresis or by fluid flow, the captured rare cells can be recovered by removing the physical force that causes them to adhere to the chip surface, and collecting the cells in a vessel using fluid flow.

Combined Solution for Sedimenting Red Blood Cells and Selectively Removing Undesirable Sample Components of a Blood Sample

In preferred embodiments of the present invention, a solution that sediments red blood cells can also include one or more additional specific binding members that can be used to selectively remove undesirable sample components other than red blood cells from the blood sample. In this regard, the present invention includes a combined sedimenting solution for enriching rare cells of a blood sample that sediments red blood cells and provides reagents for the removal of other undesirable components of the sample. Thus a combined solution for processing a blood sample comprises: dextran; at least one specific binding member that can induce agglutination of red blood cells; and at least one additional specific binding member that can specifically bind undesirable components of the sample other than RBCs.

Addition of Sedimenting Solution to Sample

A red blood cell sedimenting solution can be added to a blood sample by any convenient means, such as pipeting, automatic liquid uptake/dispensing devices or systems, pumping through conduits, etc. The amount of sedimenting solution that is added to a blood sample can vary, and will largely be determined by the concentration of dextran and specific binding members in the sedimenting solution (as well as other components), so that their concentrations will be optimal when mixed with the blood sample. Optimally, the volume of a blood sample is assessed, and an appropriate proportional volume of sedimenting solution, ranging from 0.01 to 100 times the sample volume, preferably ranging from 0.1 times to 10 times the sample volume, and more preferably from 0.25 to 5 times the sample volume, and even more preferably from 0.5 times to 2 times the sample volume, is added to the blood sample. (It is also possible to add a blood sample, or a portion thereof, to a red blood cell sedimenting solution. In this case, a known volume of sedimenting solution can be provided in a tube or other vessel, and a measured volume of a blood sample can be added to the sedimenting solution.)

Specific Binding Member for Removing Undesirable Components

In addition to the components of a sedimenting solution of the present invention, a combined solution of the present invention can comprise at least one specific binding member that can selectively bind undesirable components of a blood sample (including but not limited to red blood cells, white blood cells, platelets, serum proteins) and have less binding to desirable components. One or more specific binding members that can selectively bind undesirable components of a sample can be used to remove the undesirable components of the sample, increasing the relative proportion of rare cells in the sample, and thus contribute to the enrichment of rare cells of the sample. By “selectively binds” is meant that a specific binding member used in the methods of the present invention to remove one or more undesirable sample components does not appreciably bind to desirable cells of the sample. By “does not appreciably bind” is meant that not more than 30%, preferably not more than 10%, and more preferably not more than 1.0% of one or more desirable cells are bound by the specific binding member used to remove undesirable components from the sample. In many cases, the undesirable components of a blood sample will be white blood cells. In preferred embodiments of the present invention, a combined solution of the present invention can be used for sedimenting red blood cells and selectively removing white blood cells from a blood sample.

A specific binding member that can specifically bind white blood cells can be as nonlimiting examples, an antibody, a ligand for a receptor, transporter, channel or other moiety of the surface of a white blood cell, or a lectin or other protein that can specifically bind particular carbohydrate moieties on the surface of a white blood cell (for example, sulfated Lewis-type carbohydrates, glycolipids, proteoglycans or selectin).

Preferably, a specific binding member that selectively binds white blood cells is an antibody that binds white blood cells but does not appreciably bind fetal nucleated cells, such as, for example, an antibody to CD3, CD11b, CD14, CD17, CD31, CD45, CD50, CD53, CD63, CD69, CD81, CD84, CD102, or CD166. Antibodies can be purchased commercially from suppliers such as, for example Dako, BD Pharmingen, Antigenix America, Neomarkers, Leinco Technologies, Research & Diagnostic Systems, Serotec, United States Biological, Bender Medsystems Diagnostics, Ancell, Leinco Technologies, Cortex Biochem, CalTag, Biodesign, Biomeda, Accurate Chemicals & Scientific and Chemicon International. Antibodies can be tested for their ability to bind an efficiently remove white blood cells and allow for the enrichment of desirable cells from a sample using capture assays well known in the art.

Specific binding members that selectively bind to one or more undesirable components of the present invention can be used to capture one or more undesirable components, such that one or more desirable components of the fluid sample can be removed from the area or vessel where the undesirable components are bound. In this way, the undesirable components can be separated from other components of the sample that include the rare cells to be separated. The capture can be affected by attaching the specific binding members that recognize the undesirable component or components to a solid support, or by binding secondary specific binding members that recognize the specific binding members that bind the undesirable component or components, to a solid support, such that the undesirable components become attached to the solid support. In preferred embodiments of the present invention, specific binding members that selectively bind undesirable sample components provided in a combined solution of the present invention are coupled to a solid support, such as microparticles, but this is not a requirement of the present invention.

Magnetic beads are preferred solid supports for use in the methods of the present invention to which specific binding members that selectively bind undesirable sample components can be coupled. Magnetic beads are known in the art, and are available commercially. Methods of coupling molecules, including proteins such as antibodies, lectins and avidin and its derivatives, to microparticles such as magnetic beads are known in the art. Preferred magnetic beads of the present invention are from 0.02 to 20 microns in diameter, preferably from 0.05 to 10 microns in diameter, and more preferably from 0.05 to 5 microns in diameter, and even more preferably from 0.05 to 3 microns in diameter and are preferably provided in a combined solution of the present invention coated with a primary specific binding member, such as an antibody that can bind a cell that is to be removed from the sample, or a secondary specific binding member, such as streptavidin or neutravidin, that can bind primary specific binding members that bind undesirable sample components (such as biotinylated primary specific binding members).

In preferred embodiments of the present invention, the fluid sample is a maternal blood sample, the rare cells whose separation are desirable are fetal cells, and the undesirable components of the sample to be removed from the sample are white blood cells and other serum components. In these embodiments, a specific binding member that selectively binds white blood cells is used to remove the white blood cells from the sample by magnetic capture. Preferably, the specific binding member provided is attached to magnetic beads for direct capture, or, is provided in biotinylated form for indirect capture of white blood cells by streptavidin-coated magnetic beads.

A combined solution for enriching rare cells of a blood sample of the present invention can also include other components, such as, but not limited to, salts, buffering agents, agents for maintaining a particular osmolality, chelators, proteins, lipids, small molecules, anticoagulants, etc. For example, in some preferred aspects of the present invention, a combined solution comprises physiological salt solutions, such as PBS, PBS lacking calcium and magnesium or Hank's balanced salt solution. In some preferred aspects of the present invention, EDTA or heparin or ACD are present to prevent red blood cell clotting.

Mixing

The blood sample and red blood cell sedimenting solution are mixed so that the chemical RBC aggregating agent (such as a polymer, such as, for example, dextran) and one or more specific binding members of the sedimenting solution, as well as the components of the blood sample are distributed throughout the sample vessel. Mixing can be achieved means such as electrically powered acoustic mixing, stirring, rocking, inversion, agitation, etc., with methods such as rocking and inversion, that are least likely to disrupt cells, being favored.

Incubation of Blood Sample and Sedimenting Solution

The sample mixed with sedimenting solution is allowed to incubate to allow red blood cells to sediment. Preferably the vessel comprising the sample is stationary during the sedimentation period so that the cells can settle efficiently. Sedimentation can be performed at any temperature from about 5° C. to about 37° C. In most cases, it is convenient to perform the steps of the method from about 15° C. to about 27° C. The optimal time for the sedimentation incubation can be determined empirically for a given sedimenting solution, while varying such parameters as the concentration of dextran and specific binding members in the solution, the dilution factor of the blood sample after adding the sedimenting solution, and the temperature of incubation. Preferably, the sedimentation incubation is from five minutes to twenty four hours in length, more preferably from ten minutes to four hours in length, and most preferably from about fifteen minutes to about one hour in length. In some preferred aspects of the present invention, the incubation period is about thirty minutes.

### IV. Methods of Using Automated Filtration Unit for Separating a Target Component of a Fluid Sample

In still another aspect, the present invention also includes methods of enriching and analyzing a component in a fluid sample using the automated system disclosed herein, comprising: a) dispensing the fluid sample into the filtration chamber; b) providing a fluid flow of the fluid sample through the antechamber of the filtration chamber and a fluid flow of a solution through the post-filtration subchamber of the filtration chamber, wherein the target component of the fluid sample is retained or flows through the filter; and c) analyzing the labeled target component using the analysis apparatus.

### V. Exemplary Embodiments

1. A filtration chamber comprising a microfabricated filter enclosed in a housing, wherein the filtration chamber comprises an antechamber and a post-filtration subchamber, and the fluid flow path in the antechamber is substantially opposite to the fluid flow path in the post-filtration subchamber.

2. The filtration chamber of embodiment 1, wherein each of the antechamber and the post-filtration subchamber has an inflow port and/or an outflow port.

3. The filtration chamber of embodiment 2, wherein the antechamber comprises at least two inflow ports.

4. The filtration chamber of embodiment 3, wherein the antechamber comprises a suprafilter thereby creating a suprachamber.

5. The filtration chamber of embodiment 4, wherein the suprafilter, between the antechamber and the suprachamber, is sufficiently rigid to maintain its flatness under slow flow conditions.

6. The filtration chamber according to embodiment 4 or 5, wherein the suprafilter comprises holes or slots with openings smaller than about 5 microns.

7. The filtration chamber according to any one of embodiments 2-6, wherein the inflow port and outflow port may be used interchangeably.

8. The filtration chamber according to any one of embodiments 1-7, wherein the microfabricated filter comprises one or more tapered slots.

9. The filtration chamber of embodiment 8, wherein the microfabricated filter comprises from about 100 to 5,000,000 tapered slots.

10. The filtration chamber according to any one of embodiments 1-9, wherein the thickness of the microfabricated filter is from about 20 to about 200 microns.

11. The filtration chamber of embodiment 10, wherein the thickness of the microfabricated filter is from about 40 to about 70 microns.

12. The filtration chamber according to any one of embodiments 8-11, wherein the tapered slots are from approximately 20 microns to 200 microns in length and from about 2 microns to about 16 microns in width, and the tapering of said slots is from about 0 degree to about 10 degrees, and wherein the variation in slot size of said tapered slot is less than about 20%.

13. The filtration chamber according to any one of embodiments 8-11, wherein the size of the tapered slots varies by more than 20%.

14. The filtration chamber of embodiment 13, wherein the size of the tapered slots varies by more than 50%.

15. The filtration chamber of embodiment 14, wherein the size of the tapered slots varies by more than 100%.

16. The filtration chamber according to any one of embodiments 13-15, wherein the size of the tapered slots varies along the fluid flow path in the antechamber.

17. The filtration chamber according to any one of embodiments 2-16, wherein the post-filtration subchamber comprises at least two outflow ports.

18. The filtration chamber of embodiment 17, wherein the at least two outflow ports are arranged along the fluid flow path in the antechamber.

19. The filtration chamber according to any one of embodiments 1-18, comprising two or more electrodes.

20. The filtration chamber of embodiment 19, wherein the electrodes are placed on opposite sides of the microfabricated filter.

21. The filtration chamber according to embodiment 19 or 20, wherein the electrodes are placed on the housing of the filtration chamber.

22. The filtration chamber according to any one of embodiments 19-21, wherein the electrodes are placed in the antechamber and/or the post-filtration subchamber.

23. The filtration chamber according to any one of embodiments 19-21, wherein the electrodes are incorporated or placed into one or more of the ports or connections that interact with the antechamber and/or the post-filtration subchamber.

24. The filtration chamber according to any one of embodiments 1-23, wherein the filtration chamber comprises at least one acoustic element.

25. The filtration chamber according to any one of embodiments 1-24, wherein the outflow port of the antechamber is connected to a collection chamber or collection well.

26. The filtration chamber according to any one of embodiments 1-25, wherein the housing comprises a top part and a bottom part, and the top part and the bottom part engage or bond together to form the filtration chamber.

27. The filtration chamber according to any one of embodiments 1-26, wherein the filtration chamber has a length of about 1 mm to about 10 cm, a width of about 1 mm to about 3 cm, and a depth of about 0.02 mm to about 20 mm.

28. The filtration chamber of embodiment 27, wherein the filtration chamber has a length of about 10 mm to about 50 mm, a width of about 5 mm to about 20 mm, and a depth of about 0.05 mm to about 2.5 mm.

29. The filtration chamber of embodiment 28, wherein the filtration chamber has a length of about 30 mm, a width of about 6 mm, and a depth of about 1 mm.

30. The filtration chamber according to any one of embodiments 1-29, wherein the housing has a length of about 38 mm, a width of about 12 mm, and a depth of about 20 mm as outer dimensions.

31. The filtration chamber according to any one of embodiments 27-30, wherein its antechamber has a length of about 1 mm to about 10 cm, a width of about 1 mm to about 3 cm, and a depth of about 0.01 mm to about 10 mm.

32. The filtration chamber of embodiment 31, wherein its antechamber has a length of about 10 mm to about 50 mm, a width of about 5 mm to about 20 mm, and a depth of about 0.01 mm to about 1 mm.

33. The filtration chamber of embodiment 32, wherein its antechamber has a length of about 30 mm, a width of about 6 mm, and a depth of about 0.1-0.4 mm.

34. The filtration chamber according to any one of embodiments 31-33, wherein the volume of the antechamber is about 0.01 μL to about 5 mL.

35. The filtration chamber of embodiment 34, wherein the volume of the antechamber is about 1 μL to about 100 μL.

36. The filtration chamber of embodiment 35, wherein the volume of the antechamber is about 40 to 80 μL.

37. The filtration chamber according to any one of embodiments 27-36, wherein the post-filtration subchamber has a length of about 1 mm to about 10 cm, a width of about 1 mm to about 3 cm, and a depth of about 0.01 mm to about 1 cm.

38. The filtration chamber of embodiment 37, wherein the post-filtration subchamber has a length of about 10 mm to about 50 mm, a width of about 5 mm to about 20 mm, and a depth of about 0.2 mm to about 1.5 mm.

39. The filtration chamber of embodiment 38, wherein the post-filtration subchamber has a length of about 30 mm, a width of about 6.4 mm, and a depth of about 0.6-1 mm.

40. A filtration chamber comprising a microfabricated filter enclosed in a housing, wherein the surface of said filter and/or the inner surface of said housing are modified by vapor deposition, sublimation, vapor-phase surface reaction, or particle sputtering to produce a uniform coating.

41. The filtration chamber of embodiment 40, wherein the filtration chamber comprises an antechamber and a post-filtration subchamber.

42. The filtration chamber of embodiment 41, wherein the antechamber comprises a suprafilter thereby creating a suprachamber.

43. The filtration chamber of embodiment 42, wherein the surface of the suprafilter is modified by vapor deposition, sublimation, vapor-phase surface reaction, or particle sputtering to produce a uniform coating.

44. The filtration chamber according to any one of embodiments 40-43, wherein the modification is by physical vapor deposition.

45. The filtration chamber according to any one of embodiments 40-43, wherein the modification is by plasma-enhanced chemical vapor deposition.

46. The filtration chamber according to any one of embodiments 40-43, wherein the vapor deposition is of a metal nitride or a metal halide.

47. The filtration chamber of embodiment 46, wherein the metal nitride is titanium nitride, silicon nitride, zinc nitride, indium nitride, and/or boron nitride.

48. The filtration chamber according to any one of embodiments 40-43, wherein the modification is by chemical vapor deposition.

49. The filtration chamber of embodiment 48, wherein the chemical vapor deposition is by a Parylene or derivative thereof.

50. The filtration chamber of embodiment 49, wherein the Parylene or derivative thereof is selected from the group consisting of Parylene, Parylene-N, Parylene-D, Parylene AF-4, Parylene SF, and Parylene HT.

51. The filtration chamber of embodiment 48, wherein the modification is by polytetrafluoroethylene (PTFE).

52. The filtration chamber of embodiment 48, wherein the modification is by Teflon-AF.

53. The filtration chamber according to embodiment 40 or 43, wherein the modification is by a perfluorocarbon.

54. The filtration chamber of embodiment 53, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane and is in liquid form.

55. The filtration chamber according to any one of embodiments 40-54, wherein the filter and/or housing comprises silicon, silicon dioxide, glass, metal, carbon, ceramics, plastic, or a polymer.

56. The filtration chamber according to any one of embodiments 40-54, wherein the filter and/or housing comprises silicon nitride or boron nitride.

57. A filtration chamber comprising a microfabricated filter enclosed in a housing, wherein the surface of said filter and/or the inner surface of said housing are modified by a metal nitride, a metal halide, a Parylene or derivative thereof, a polytetrafluoroethylene (PTFE), a Teflon-AF or a perfluorocarbon.

58. The filtration chamber of embodiment 57, wherein the filtration chamber comprises an antechamber and a post-filtration subchamber.

59. The filtration chamber of embodiment 58, wherein the antechamber comprises a suprafilter thereby creating a suprachamber.

60. The filtration chamber of embodiment 59, wherein the surface of the suprafilter is modified by a metal nitride, a metal halide, a Parylene or derivative thereof, a polytetrafluoroethylene (PTFE), a Teflon-AF or a perfluorocarbon.

61. The filtration chamber according to any one of embodiments 57-60, wherein the metal nitride is titanium nitride, silicon nitride, zinc nitride, indium nitride, and/or boron nitride.

62. The filtration chamber according to any one of embodiments 57-60, wherein the Parylene or derivative thereof is selected from the group consisting of Parylene, Parylene-N, Parylene-D, Parylene AF-4, Parylene SF, and Parylene HT.

63. The filtration chamber according to any one of embodiments 57-60, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane, and the perfluorocarbon covalently binds the surface.

64. The filtration chamber according to any one of embodiments 57-63, wherein the filter and/or housing comprises silicon, silicon dioxide, glass, metal, carbon, ceramics, plastic, or a polymer.

65. The filtration chamber according to any one of embodiments 57-63, wherein the filter and/or housing comprises silicon nitride or boron nitride.

66. A filtration chamber according to any one of embodiments 1-39, wherein the surface of the filter and/or the inner surface of said housing are modified by vapor deposition, sublimation, vapor-phase surface reaction, or particle sputtering to produce a uniform coating.

67. The filtration chamber of embodiment 66, wherein the vapor deposition is of a metal nitride or a metal halide.

68. The filtration chamber of embodiment 67, wherein the metal nitride is titanium nitride, silicon nitride, zinc nitride, indium nitride, and/or boron nitride.

69. The filtration chamber of embodiment 66, wherein the modification is by chemical vapor deposition.

70. The filtration chamber of embodiment 66, wherein the modification is by a perfluorocarbon.

71. The filtration chamber of embodiment 70, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane and is in liquid form.

72. A filtration chamber according to any one of embodiments 1-39, wherein the surface of the filter and/or the inner surface of said housing are modified by a metal nitride, a metal halide, a Parylene, a polytetrafluoroethylene (PTFE), a Teflon-AF or a perfluorocarbon.

73. The filtration chamber of embodiment 72, wherein the metal nitride is titanium nitride, silicon nitride, zinc nitride, indium nitride, and/or boron nitride.

74. The filtration chamber of embodiment 72, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane, and the perfluorocarbon covalently binds the surface.

75. A filtration chamber according to any one of embodiments 1-74, comprising at least two microfabricated filters.

76. The filtration chamber of embodiment 75, wherein the at least two microfabricated filters are arranged in tandem.

77. A filtration chamber comprising at least two filtration chambers according to any one of embodiments 1-76 arranged in tandem.

78. The filtration chamber of embodiment 77, wherein the antechambers of the at least two filtration chambers are in fluid connection.

79. The filtration chamber of embodiment 78, wherein the at least two filtration chambers share one microfabricated filter and/or suprafilter.

80. The filtration chamber according to embodiment 77 or 78, wherein the slots of the filters within each filtration chamber are of different widths, and the filtration chambers are arranged in order of increasing slot widths.

81. A cartridge comprising the filtration chamber according to any one of embodiments 1-80.

82. The cartridge of embodiment 81, comprising at least two filtration chambers.

83. The cartridge of embodiment 82, comprising eight filtration chambers.

84. An automated filtration unit for separating a target component in a fluid sample, comprising the filtration chamber according to any one of embodiments 1-80.

85. The automated filtration unit of embodiment 84, further comprising a control algorithm for controlling the fluid flow in the filtration chamber.

86. The automated filtration unit according to embodiment 84 or 85, comprising at least two filtration chambers.

87. The automated filtration unit according to embodiment 86, wherein the at least two filtration chambers are arranged in tandem, and the filtration chambers comprise filters of increasing slot width.

88. The automated filtration unit according to embodiment 86 or 87, wherein the filters contain slot widths of increasing size along the fluidic path.

89. The automated filtration unit of embodiment 88, comprising a suprachamber.

90. The automated filtration unit according to any one of embodiments 84-89, wherein the post-filtration subchamber comprises multiple partitions each comprising an outflow port.

91. The automated filtration of embodiment 90, wherein the outflow port from each partition of the post-filtration chamber is aligned with individual wells of a multi-well plate.

92. The automated filtration of embodiment 91, wherein the wells are spaced about every 1-100 mm.

93. The automated filtration of embodiment 91, wherein the wells are spaced about every 2.25 mm.

94. The automated filtration of embodiment 91, wherein the wells are spaced about every 4.5 mm.

95. The automated filtration of embodiment 91, wherein the wells are spaced about every 9 or 18 mm.

96. The automated filtration unit according to any one of embodiments 84-95, comprising eight filtration chambers.

97. The automated filtration unit according to any one of embodiments 84-96, comprising a means for effecting fluid flow in the filtration chamber.

98. The automated filtration unit of embodiment 97, wherein the means for effecting fluid flow is a fluidic pump.

99. The automated filtration unit according to any one of embodiments 84-98, comprising a means for collecting the separated target component.

100. An automated system for separating and analyzing a target component in a fluid sample, comprising the automated filtration unit according to any one of embodiments 84-98 and an analysis apparatus connected to the filtration unit.

101. The automated system of embodiment 100, wherein the analysis apparatus is a cell sorting device or a flow cytometer.

102. A method for separating a target component in a fluid sample, comprising:

a) dispensing the fluid sample into the filtration chamber according to any one of embodiments 1-80; and

b) providing a fluid flow of the fluid sample through the filtration chamber, wherein the target component of the fluid sample is retained by or passes through the filter.

103. The method of embodiment 102, comprising providing a fluid flow of the fluid sample through the antechamber of the filtration chamber and a fluid flow of a solution through the post-filtration subchamber of the filtration chamber, and optionally a fluid flow of a solution through the suprachamber of the filtration chamber.

104. The method according to embodiment 102 or 103, wherein the fluid sample is separated based on the size, shape, deformability, binding affinity and/or binding specificity of the components.

105. The method according to embodiment 103 or 104, wherein the fluid sample is dispensed through the inflow port of the antechamber.

106. The method according to any one of embodiments 103-105, wherein the solution is introduced to the inflow port of the post-filtration subchamber.

107. The method according to any one of embodiments 103-105, wherein the solution is introduced to the inflow port of the supra-filtration chamber.

108. The method according to any one of embodiments 102-107, wherein the fluid sample is manipulated by a physical force effected via a structure that is external to the filter and/or a structure that is built-in on the filter.

109. The method of embodiment 108, wherein the physical force is selected from the group consisting of a dielectrophoretic force, a traveling-wave dielectrophoretic force, a magnetic force, an acoustic force, an electrostatic force, a mechanical force, an optical radiation force and a thermal convection force.

110. The method of embodiment 109, wherein the dielectrophoretic force or the traveling-wave dielectrophoretic force is effected via an electrical field produced by an electrode.

111. The method of embodiment 109, wherein the acoustic force is effected via a standing-wave acoustic field or a traveling-wave acoustic field.

112. The method of embodiment 109, wherein the acoustic force is effected via an acoustic field produced by piezoelectric material.

113. The method of embodiment 109, wherein the acoustic force is effected via a voice coil or audio speaker.

114. The method of embodiment 109, wherein the electrostatic force is effected via a direct current (DC) electric field.

115. The method of embodiment 109, wherein the optical radiation force is effected via laser tweezers.

116. The method according to any one of embodiments 102-115, wherein the fluid sample is blood, effusion, urine, bone marrow sample, ascitic fluid, pelvic wash fluid, pleural fluid, spinal fluid, lymph, serum, mucus, sputum, saliva, semen, ocular fluid, extract of nasal, throat or genital swab, cell suspension from digested tissue, extract of fecal material, cultured cells of either mixed types and/or mixed sizes, or cells that contain contaminants or unbound reactants that need to be removed.

117. The method of embodiment 116, wherein the fluid sample is a blood sample and the component being removed is a plasma, a platelet and/or a red blood cell (RBC).

118. The method of embodiment 116, wherein the fluid sample are cells that contain contaminants or unbound reactants that need to be removed, and the reactant is a labeling reagent for the cells.

119. The method of embodiment 116, wherein the fluid sample is a blood sample and the target component is a nucleated cell, e.g., a non-hematopoietic cell, a subpopulation of blood cells, a fetal red blood cell, a stem cell, or a cancerous cell.

120. The method of embodiment 116, wherein the fluid sample is an effusion or a urine sample and the target component is a nucleated cell, e.g., a cancerous cell or a non-hematopoietic cell.

121. A method of separating a target component in a fluid sample using the automated filtration unit according to any one of embodiments 84-99, comprising:

a) dispensing the fluid sample into the filtration chamber; and

b) providing a fluid flow of the fluid sample through the filtration chamber, wherein the target component of the fluid sample is retained by or flows through the filter.

122. The method of embodiment 121, wherein the fluid sample is separated based on the size, shape, deformability, binding affinity and/or binding specificity of the components.

123. The method according to embodiment 121 or 122, wherein the fluid sample in the antechamber flows substantially anti-parallel to the solution in the post-filtration subchamber.

124. The method according to any one of embodiments 121-123, wherein the filter rate is about 0-5 mL/min.

125. The method of embodiment 124, wherein the filter rate is about 10-500 μL/min.

126. The method of embodiment 125, wherein the filter rate is about 80-140 μL/min.

127. The method according to any one of embodiments 124-126, wherein the feed rate is about 1-10 times the filter rate.

128. The method according to any one of embodiments 102-127, further comprising:

c) rinsing the retained components of the fluid sample with an additional sample-free rinsing reagent.

129. The method of embodiment 128, wherein during the rinsing step the feed rate is less than or equal to the filter rate.

130. The method according to embodiment 128 or 129, wherein a rinsing reagent is introduced to the post-filtration subchamber.

131. The method according to embodiment 128 or 129, wherein the rinsing reagent is introduced to the antechamber and/or the suprachamber.

132. The method according to any one of embodiments 102-131, further comprising:

d) providing a labeling reagent to bind to the target component.

133. The method of embodiment 132, wherein the labeling reagent is an antibody.

134. The method according to embodiment 132 or 133, wherein the labeling reagent is added to the collection chamber.

135. The method according to embodiment 132 or 133, wherein the labeling reagent is added to the antechamber and/or the suprachamber.

136. The method according to any one of embodiments 132-135, wherein during the labeling step the fluid flow in the post-filtration subchamber is stopped.

137. The method according to any one of embodiments 132-136, further comprising:

e) removing the unbound labeling reagent.

138. The method according to any one of embodiments 102-137, further comprising:

f) recovering the target component in the collection chamber.

139. The method of embodiment 138, wherein during the recovering step the feed rate is about 5-20 mL/min.

140. The method according to embodiment 138 or 139, wherein during the recovering step the outflow rate equals the inflow rate in the post-filtration subchamber.

141. The method according to any one of embodiments 138-140, wherein during the recovering step the outflow is paused for about 50 ms.

142. The method according to any one of embodiments 121-141, wherein the fluid sample is a blood sample, which comprises removing at least one type of undesirable component using a specific binding member.

143. The method of embodiment 142, wherein the at least one undesirable component are white blood cells (WBCs).

144. The method of embodiment 143, wherein the specific binding member selectively binds to WBCs and is coupled to a solid support.

145. The method of embodiment 144, wherein the specific binding member is an antibody or an antibody fragment that selectively binds to WBCs.

146. The method of embodiment 145, wherein the specific binding member is an antibody that selectively binds to CD3, CD11b, CD14, CD17, CD31, CD45, CD50, CD53, CD63, CD69, CD81, CD84, CD102 or CD166.

147. The method of embodiment 146, wherein the specific binding member is an antibody that selectively binds to CD35 and/or CD50.

148. The method according to any one of embodiments 142-147, further comprising contacting the blood sample with a secondary specific binding member.

149. The method of embodiment 148, wherein said secondary specific binding member is an antibody that selectively binds to CD31, CD36, CD41, CD42 (a, b or c), CD51, or CD51/61.

150. A method of enriching and analyzing a target component in a fluid sample using the automated system according to embodiment 100 or 101, comprising,

a) dispensing the fluid sample into the filtration chamber;

b) providing a fluid flow of the fluid sample through the antechamber of the filtration chamber and a fluid flow of a solution through the post-filtration subchamber of the filtration chamber, wherein the target component of the fluid sample is retained in the antechamber and non-target components flow through the filter into the post-filtration subchamber;

c) labeling the target component; and

d) analyzing the labeled target component using the analysis apparatus.

151. The method of embodiment 150, comprising providing fluid flow into the suprachamber.

152. The method according to embodiment 150 or 151, wherein the target component is a cell or cellular organelle.

153. The method of embodiment 152, wherein the cell is a nucleated cell.

154. The method of embodiment 152, wherein the cell is a rare cell.

### EXAMPLES

### Example 1

### Fabrication of a Filter for Removing Red Blood Cells from a Blood Sample

A silicon chip of dimensions (1.8 cm by 1.8 cm×500 micron) was used to fabricate a filtration area of 1 cm by 1 cm by 50 micron with slots having dimensions from about 0.1 micron to about 1000 microns, preferably from about 20 to 200 microns, preferably from about 1 to 10 microns, more preferably 2.5 to 5 microns. The slots were vertically straight with a maximum tapered-angle of less than 2%, preferably less than about 0.5% with an offset distance between neighboring columns of the filter slots were 1 to 500 microns, preferably from 5 to 30 microns.

Manufacturing included providing a silicon chip having the above referenced dimensions and coating the top and bottom of the silicon chip with a dielectric layer. A cavity along the bottom portion of the chip was then created. The cavity was formed by removing an appropriate cavity pattern from the dielectric layer, and then etching the silicon chip generally following the pattern, until desired thickness is reached. The chip was re-oxidized to coat the contoured region. A filter pattern was then removed from the dielectric layer coating the top of the silicon chip in substantial alignment (above) with the cavity. The silicon chip was etched (e.g., via deep RIE or ICP processes) at the above referenced angles starting at the pattern created along the top of the chip until the silicon layer has been etched through. The dielectric layer from the top and bottom were then removed. By removing the dielectric layer within the cavity, throughbores, referred to as slots, were created. It is also possible to create these slots using laser cuts to bore though materials, including but not limited to silica or polymers such as plastic.

### Example 2

### Chemical Treatment of a Microfabricated Filter

A filter chip made as described in Example 1 was placed on a ceramic heating plate in an oven and heated at 800 degrees Celsius for 2 hours in oxygen containing gas (e.g. air). The heating source was then turned off the chips are slowly cooled overnight. This results in a thermally grown layer on the surface of the chip.

A nitride layer could also be deposited onto the filter surface. An oxide layer is put on the surface of the chip by low-pressure chemical vapor deposition (LPCVD) in a reactor at temperatures up to ˜900° C. The deposited film is a product of a chemical reaction between the source gases supplied to the reactor. The process is typically performed on both sides of the substrate at the same time to form a layer of Si3N4.

### Example 3

### Polyvinylpyrrolidone (PVP) and Polyvinyl Alcohol (PVA) Filter Coatings

Filter chips made by the method of Example 1 were coated with either PVP or PVA. For coating the chips with either PVP or PVA, the chips were pre-treated as follows: The filter chips were rinsed with deionized water and then immersed in 6N nitric acid. The chips were placed on a shaker for 30 minutes at 50 degrees Celsius. After acid treatment, the chips were rinsed in deionized water.

For PVP coating, chips were immersed in 0.25% polyvinylpyrrolidone (K-30) at room temperature until the chips were ready for use. Chips were then rinsed with deionized water and dried by pressurized air.

For PVA coating, after acid treatment and rinsing in water, the chips were stored in water prior to coating. To make the 0.25% PVA (Mn 35,000-50,000) solution, dissolve the PVA in water under slow heating to 80 degrees Celsius and gentle stirring. To coat, the chips were immersed in a hot PVA solution and heated for 1-2 hours. The chips were then rinsed in deionized water and dried by pressurized air.

### Example 4

### Bovine Serum Albumin (BSA) Filter Coating

For coating filter chips with BSA, the chips were pre-treated as follows: The filter chips were rinsed with deionized water and then immersed in 95% ethanol for 10 seconds at room temperature and then were rinsed again in deionized water.

The chips were then immersed in 2.% BSA in PBS for 2 minutes at room temperature. Chips were then rinsed with deionized water and dried by pressurized air.

### Example 5

### PEG Filter Coating

To conjugate PEG to the chip surfaces, filter chips were immersed in a solution of DBE-814 (a PEG solution containing polysiloxane from Gelest, Morrisville, Pa.) in 5% methylene chloride. The immersed chips were heated at 70 degrees Celsius for 3 hours under vacuum. After the incubation, the PEG-coated chips were rinsed in deionized water and dried by pressurized air.

### Example 6

### Process Flow Chart for Enriching Nucleated Fetal Cells from Maternal Blood

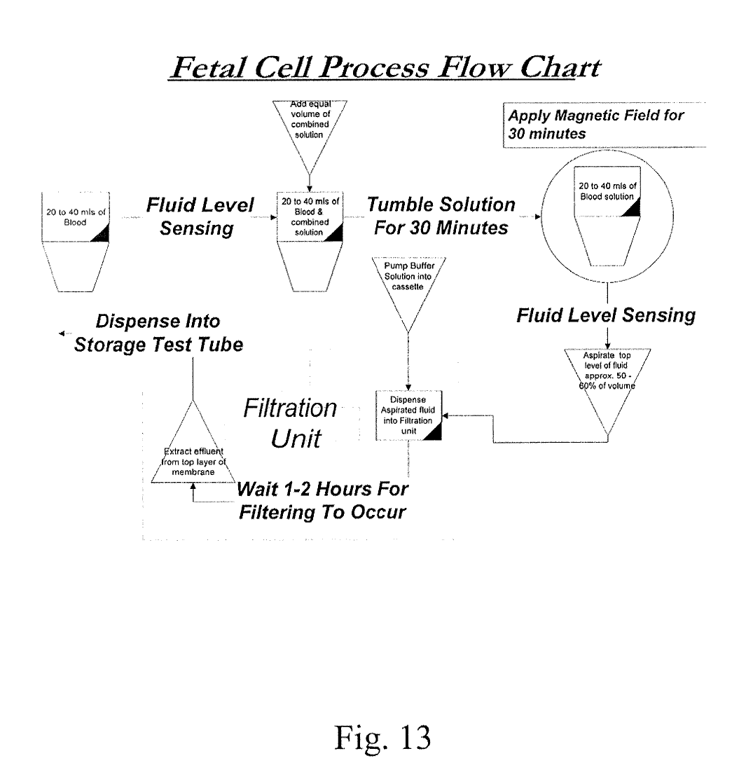

FIG. 13 shows a process flow chart for enriching fetal nucleated cells from maternal blood samples. The whole process comprises the flowing steps:
(1) The blood sample may be transferred to a centrifuge tube.
(2) The sample does not have to be but can be washed before addition to the automated unit.
(3) The process starts with a volume of blood sample 10 mls (range of 3-40 ml) in a tube(s).

Fluidic level sensing step is used to determine the exact volume of the blood sample in the tube to be processed.

Add a volume of the combined reagent (for example, an equal volume of the reagent described in Example 6) to the blood sample in the tube.

Rotate/shake/tumble/mix the solution for a period of time 0.5 hrs (range of 0.1-2 hrs).

Let the solutions in the tube settle upright for 30 minutes (range of 0.1 to 2 hrs) so that the aggregated RBCs can settle to the bottom of the tube. Simultaneously during this period, a magnetic field is applied to collect and attract magnetic beads (which may or may not have bound blood components) to a side of tube.

Another fluidic level sensing step is applied to determine what the volume of the “un-aggregated” cell suspension is present in the tube.

Aspirate appropriate volume of the fluid from the tube into the fetal cell filtration chamber (or fetal cell cassette process).

Filter the sample for 0.2-2 hr in the fetal cell filtration chamber/cassette (Further details of the filtration process are included in [Example 8], below.)

Extract solution from the top chamber of the filtration cassette and dispense into storage test tube.

### Example 7

### Process Flow Chart for Silicon Membrane Filtration Process

FIG. 14 provides a schematic diagram showing the microfiltration process. The simplified process steps include the following:
(1) Close valves B&D, open valves A&C.
(2) Test sample (coming from the first step of the procedure in [Example 9]) is loaded into the 45 mL loading reservoir.
(3) Operate waste pump for 1 h so that the sample loaded in the storage reservoir is filtered through the microfabricated filter.
(4) Apply 1-10 mL wash solution to the Loading Reservoir.
(5) Close valve A, open valve B.
(6) Wash the bottom subchamber with 1-5 mL.
(7) Close valve C and open valve D.
(8) Rotate the Cassette and filtration chamber 180 degrees.
(9) Flush the filter from valve B.
(10) Collect volume from valve D.

### Example 8

### Use of an Automated System to Isolate Fetal Cells from Maternal Blood

Ten milliliters blood samples of pregnant women (from six to thirty weeks gestation) are washed by diluting the samples with PBE and centrifuged at 470×g for 6 minutes (range of 50-900×g for 3-20 minutes). The supernatants are aspirated off, and PBE is added to the pellets and mixed. The samples are again centrifuged and the supernatants aspirated off. The final pellets are resuspended to the original volume with PBE. Ten milliliters of Combined Reagent (PBS lacking calcium and magnesium containing: 5 millimolar EDTA, 2% dextran (molecular weight from 70 to 200 kilodaltons), 0.05 micrograms (range of 0.01 to ugs) per milliliter of IgM antibodies to glycophorin A, and approximately 1-10×10⁹ pre-coated magnetic beads are manually added to the sample tubes.

The Rare Cell Isolation Automated System has control circuits for automated processing steps, and plugs into a 110 volt outlet. The tubes containing the samples are placed in a rack of a Rare Cell Isolation Automated System. The tubes are automatically rotated in the Automated System rack for 30 minutes (range between 5 and 120 minutes). The tubes are then allowed to stand upright while a second rack that has a magnet field, which is automatically positioned next to the tube rack. It is also possible to have other types of magnetic fields including but not limited to electromagnetic fields. The tubes are held in the upright position for 30 minutes (range of 5-120 minutes) so that the aggregated RBCs can settle to the bottom of the tube and WBC-magnetic bead aggregates are attracted to the side of each tube that is adjacent to the magnet. After the cells are allowed to settle, the supernatant volume is determined by the automated system using a light transmission-light sensor transparency measuring device.

The transparency measuring device consists of bars that each have a collated light source (the number of bars corresponds to the number of tubes) that can be focused on a sample tube, and a light detector that is positioned on the opposite side of the tube. The light source uses a laser beam that emits light in the infrared range (780 nanometers) and has an intensity greater than 3 milli-watts. The light from the source is focused through the sample tube, and at the other side of the sample tube the light detector having an intensity measurement device records the amount of light that has passed through the sample (the laser output measurement). The bars having the low wattage laser sources and light detectors move upward from a level at the bottom of the tubes. As each laser makes initial contact with the aggregated cells in the corresponding tube, the laser output measurement is zeroed. When the measured intensity for a given tube begins to rise above a threshold valve the vertical movement of the bar stops. The bar then moves to find the exact vertical point at which the transmitted light equals the threshold value. In this way the vertical point position of the aggregated cell/cell supernatant interface is determined. Once this level has been determined, the fluid handling unit moves to a preset location and uses a capacitive sensing routine to find the level of the bar (corresponding to the level of the interface). Using this data, the fluid handling accurately removes the supernatant from the fluid container. The supernatant is automatically dispensed directly into the loading reservoir of the filtration unit.

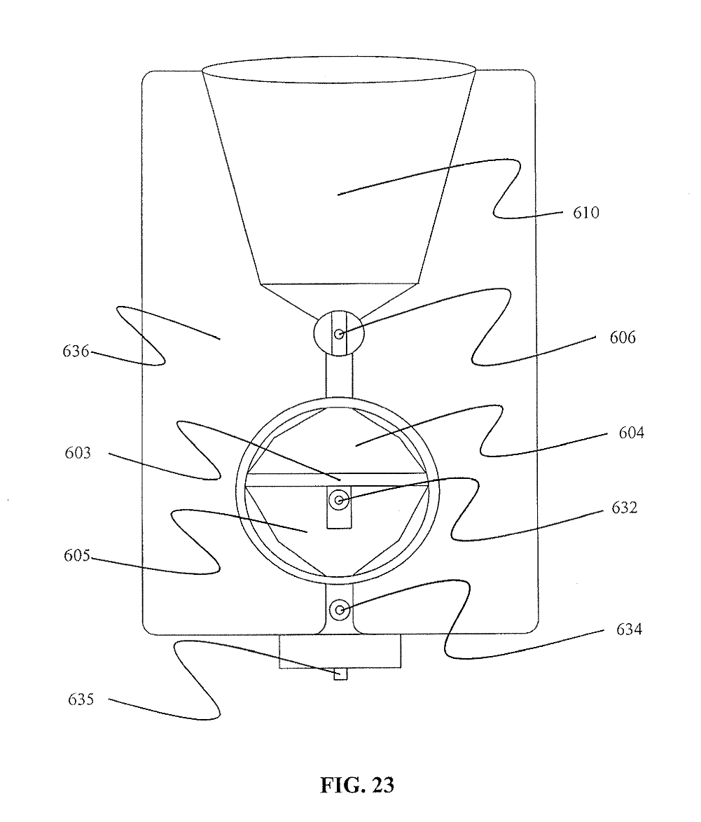

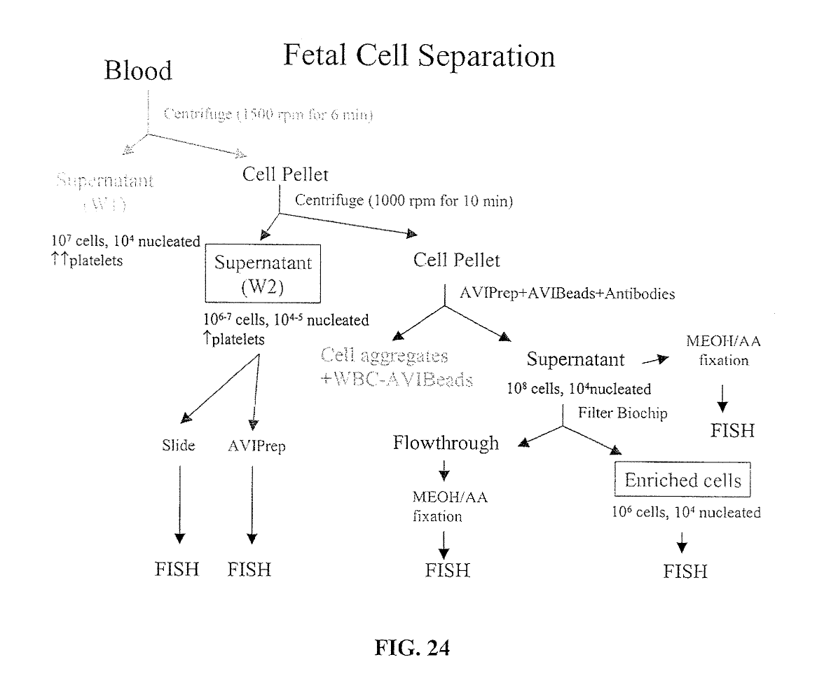

The following description of the automated separation process performed by the Rare Cell Isolation Automated System uses a filtration unit (filtration chamber, loading reservoir, and associated ports and valves) as depicted in FIG. 23. In this design, the filtration chamber can rotate 180 degrees or more within the filtration unit.

The filtration chamber comprises an antechamber (604) and a postfiltration subchamber (605) separated by a single filter (603). The microfabricated filter measuring 1.8 cm by 1.8 cm and having a filtration area of approximately 1 cm by 1 cm. The filter has approximately 94,000 slots arranged in a parallel configuration as shown in FIG. 2 with the slots having a taper of one to two degrees and dimensions of 3 microns×100 microns, within a 10% variation in each dimension. The filter slots can have dimensions of 1-10 microns by 10-500 microns with a vertical taper of 0.2 to 10 degrees depending on the target. The thickness of the filter is 50 microns (range of 10-200 microns). The filter is positioned in a two piece filtration chamber with the top half (antechamber) being an approximately rectangular filtration antechamber that tapers upward with a volume of approximately 0.5 milliliters. The bottom post-filtration subchamber is also approximately circular and tapers toward the bottom, also having a volume of approximately 0.5 milliliters. The filter covers essentially the entire bottom area of the (top) antechamber and essentially the entire top area of the (bottom) post-filtration subchamber.

In addition to the filtration chamber, the filtration unit comprises a “frame” having a loading reservoir (610), a valve controlling the flow of sample form the loading reservoir into the filtration chamber (“valve A”, 606), and separate ports for the outflow of waste or filtered sample (the waste port, 634) and for the collection of enriched rare cells (the collection port, 635). The post-filtration subchamber (605) comprises a side port (632) that can be used for the addition of buffer, and an outlet that can engage the waste port during filtration for the outflow of waste (or filtered sample). The antechamber (604) comprises an inlet that during filtration can engage the sample loading valve (valve A, 606) and during collection of enriched cells, can engage the collection port (635). During operation of an automated system, the filtration chamber (comprising the antechamber (604), post-filtration subchamber (605), and side port (632)) resides in the frame of the filtration unit.

During filtration, valve A is open, and the outlet of the post-filtration subchamber is aligned with the waste port, allowing a flow path for filtering sample from the loading reservoir through the filtration chamber and to the waste. A syringe pump draws fluid through the chamber at a flow rate of from about 10 to 500 milliliters per hour, depending upon the process step.

Prior to dispensing the appropriate volume of supernatant from each tube into the loading reservoir of the filtration unit, the side port (632) and waste port (634) of the filtration unit are closed, and valve A (606) is opened (see FIG. 23). (When the filtration unit is in the loading/filtering position, the filtration chamber does not engage the collection port (635)). With the side port of the filtration unit open, the unit is filled with PBE from the side port until the buffer reaches the bottom of the sample reservoir. The side port is then closed, and the blood sample supernatant is loaded into the loading reservoir.

Although the Rare Cell Isolation Automated System can separate several samples simultaneously, for clarity, the description of the separation process that follows will describe the filtration of a single sample. To filter a sample, the waste port (634) of a filtration unit is opened, and, using a syringe pump connected through tubing to the waste port, sample supernatant is drawn into and through the filtration chamber. As sample goes through the chamber, the larger cells stay in the top chamber (antechamber) and the smaller cells go through the filter into the lower chamber (post-filtration subchamber) and then through the waste port to the waste. Filtering is performed at a rate of approximately 2-100 milliliters per hour.

After a sample has gone through a filtration chamber (typically after from one half to two hours of filtering), three to five milliliters of PBE are added to the loading reservoir (with valve A remaining open) and pulled through the filtration chamber using the syringe pump connected to the waste port to wash the antechamber and make sure virtually all small cells are washed through.

Valve A (606) is then closed and the side port (632) is opened. Five to ten milliliters of buffer are added from the side port (632) using a syringe pump connected to tubing that is attached to the waste port (634) to wash the bottom post-filtration subchamber. After residual cells have been washed from the post-filtration subchamber (605), the bottom (post-filtration) subchamber is further cleaned by pushing air through the side port (632).

The filter cartridge is then rotated approximately 180 degrees within the filtration unit, so that the antechamber (604) is below the post-filtration subchamber (605). When the chamber rotates into collection position, the outlet of the post-filtration subchamber disengages from the waste port and, as the post-filtration subchamber becomes positioned above the antechamber, the “outlet” becomes positioned at the top of the inverted filtration chamber, but does not engage any openings in the filtration unit, and thus is blocked. As this happens, the antechamber rotates to the bottom of the inverted filtration unit, so that the antechamber inlet disengages from valve A, and instead engages the collection port at the bottom of the filtration unit. During this rotation from the filtering position to the collection position, the side port does not change position. It is aligned with the axis of rotation of the filtration chamber, and remains part of, and a functional port of, the post-filtration subchamber. As a result of this rotation, the filtration chamber is in the collection position. Thus, in the collection position, the post-filtration subchamber, having a side port but now closed off at its outlet, is above the antechamber. The antechamber “inlet” is aligned with and open to the collection port.

Approximately two milliliters of buffer is pumped into the filtration chamber through the side port to collect the cells left in the antechamber. The cells are collected into a vial that attaches to the filtration unit at the site of the sample collection port, or via tubing that leads from the sample collection port and dispenses the sample into a collection tube. Approximately 2 milliliters of additional PBE, and approximately 2 to 5 milliliters of air, is pumped through the side port to clean residual cells off of the filter and into the collection vial.

The enriched rare cells can be analyzed microscopically or using any of a number of assays, or can be stored or put into culture.

### Example 9

### Improved Magnet Configurations for Magnetic Particle Capture

To improve the efficiency of separating components such as cells from liquid samples using capture of magnetic particles to one portion of a tube or other container, several magnet configurations were tested.

Magnets of dimensions 9/16×1.25×⅛″, (Forcefield (Fort Collins, Co) NdFeB block, item #27, Nickel Plate, Br max 12,100 Gauss, Bh max 35 MGOe) were used to test the magnetic field strength. In these experiments, the strongest field could be used to capture magnetic beads that were coated with antibodies that specifically bound white blood cells, and improve the removal of white blood cells from a blood sample compared to commercially available magnetic cell separation unit MPC-1 (Dynal, Brown Deer, Wis.).

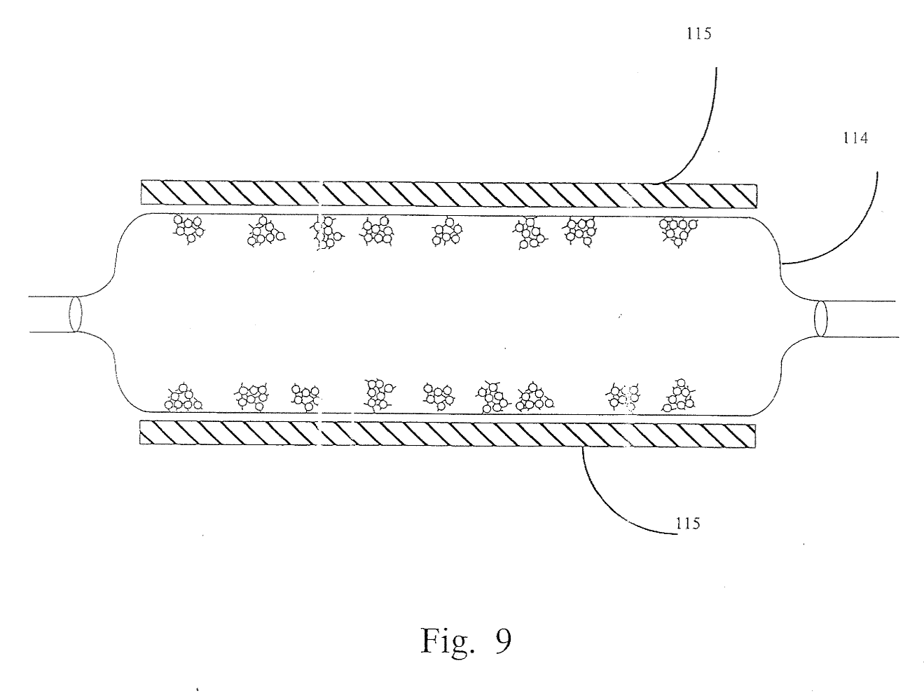

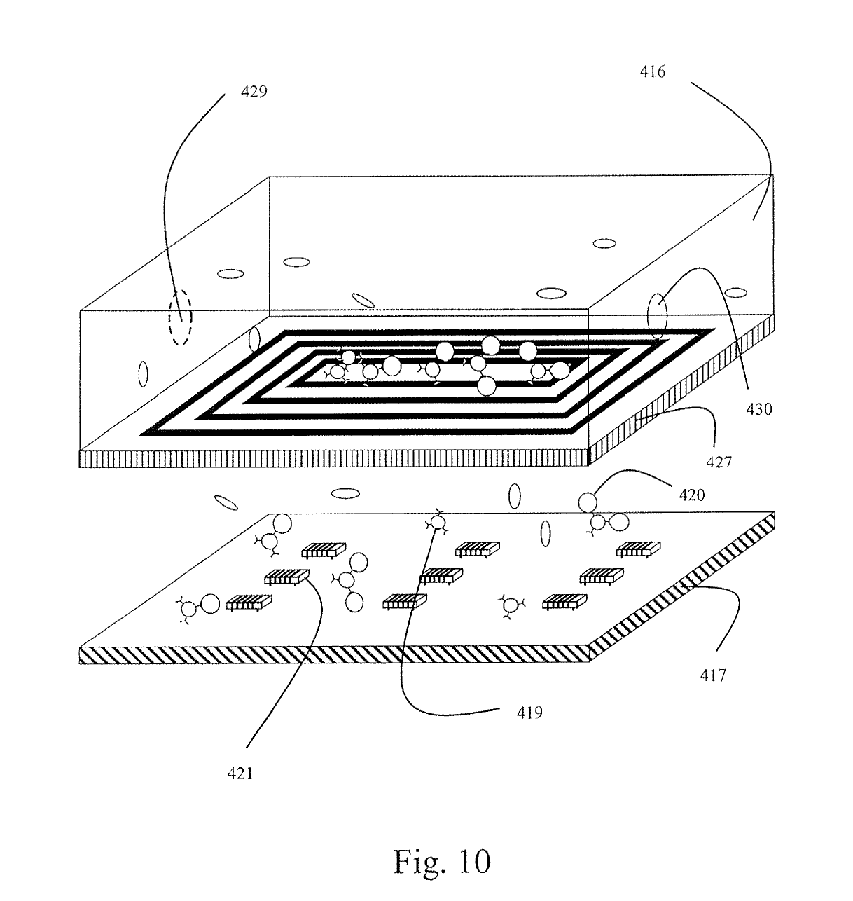

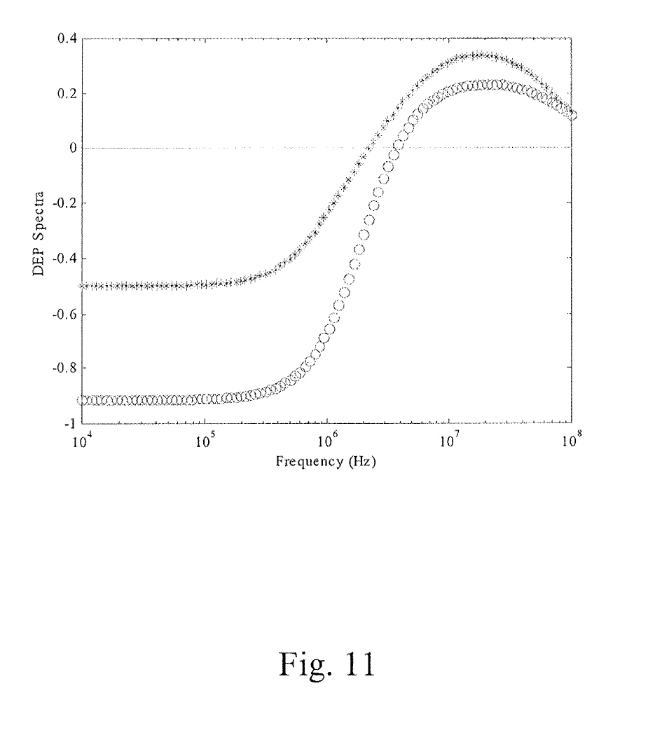

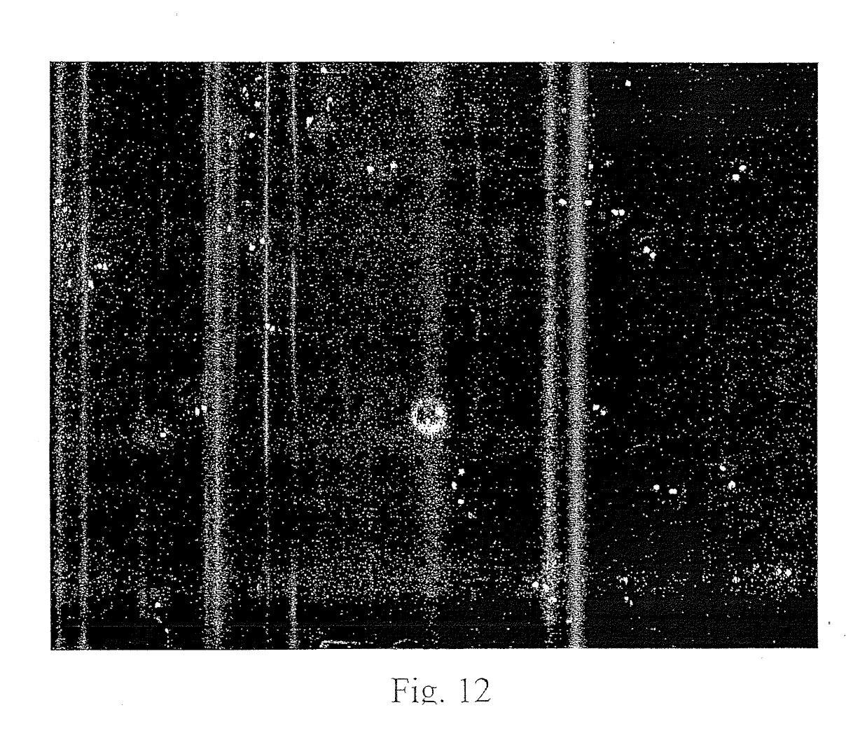

Magnets were attached in several configurations and orientations to a polypropylene stand designed to hold a 50 milliliter tube, as depicted schematically in FIG. 9. The magnetic field in the right, center, and left of the tube was measured by Gauss meter (JobMaster Magnets (Randallstown, Md.) Model GM1 using probe model PT-70, Cal #373).

### Example 10

### Whole Blood Leukocytes Isolation with Microfabricated Filter for Cell Analysis

Leukocytes carry diagnostic information about the health of immune system and are the primary samples analyzed by flow cytometry and other cell analyzers. When preparing whole blood samples for flow cytometer analysis, leukocytes are first stained with a fluorescently labeled monoclonal antibody, and then the labeled leukocytes are separated from the erythrocytes. Traditionally, separation of blood cells is performed by density gradient centrifugation, and lately, lysis of erythrocytes has become a routinely used method.

FICOLL™ HYPAQUE™ density gradient centrifugation exploits the density difference between mononuclear cells from other elements in blood fluid to perform this separation (Boyum A. Scand J Clin Lab Invest (1968) 21 (Suppl 97):77-89). Different cell populations are distributed in the ficoll solution after centrifugation in different layers based on their density. Thus mononuclear cells can be purified by collecting cells in that particular layer. The BD Vacutainer® (Becton Dickinson, Franklin Lakes, N.J.) CPT™ Cell Preparation Tube with Sodium Citrate simplifies the FICOLL HYPAQUE method, and it combines a blood collection tube containing a citrate anticoagulant with a FICOLL HYPAQUE density fluid and a polyester gel barrier that separates the two liquids. However, internal studies have shown that as many as 7% of the leukocytes are lost even during careful centrifugation steps (data not shown) and the mononuclear cell band may get disturbed due to sample sources or centrifugation process; thus desired purity can not be achieved even with the CPT tubes (Product information on BD Vacutainer® CPT™ Cell Preparation Tube with Sodium Citrate).

Whole blood lysis methods have replaced density gradients separation in many sample preparation protocols. Although there are many commercially available lysis reagents, BD FACS lysing solution is one of the standard reagents used in both Lyse Wash and Lyse No Wash assays. However, it has been reported that lysis reagents may produce artifacts when used to isolate leukocytes (Macey et al., Cytometry (1999) 38:153-160). The presence of free hemoglobin after erythrocytes lysis may also alter leukocytes' property by stimulating them to release certain cytokines (McFaul et al., Blood (1994) 84:3175-3181).

Membrane filters are applied widely in sample cleanup as they can remove particles or molecules based on size. However, classical filter membranes do not have homogeneous and precisely controlled pore sizes, so the resolving power of this separation is limited and provides only quantitative results. With classical filters, particles retained by the filter are rarely recovered in high yield. For example, filter membranes used in preparation of RNA from whole blood retain leukocytes on top of the filter, while erythrocytes pass through. However, the leukocytes are lysed on the filter without being recollected and the RNA is retained on the filter membrane (Applied Biosystems, Instruction Manual: LeukoLOCK™ Total RNA Isolation System; Life Technologies). Recently, a filter-based technology for mononuclear cells enrichment has been marketed, but recovery of mononuclear cells is only 70% (PALL Medica. Application Note: Performance Characterization of the Purecell™ Select System for Enrichment of Mononuclear Cells from Human Whole Blood; Pall Medical-Cell Therapy.).

It is desirable to have a sample preparation technology for cell analysis, which removes erythrocytes completely from leukocytes and recovers leukocytes in high yield, >95%, with no subpopulation bias. We present an evaluation of the performance characteristics of a microfabricated silicon filter device for preparing white blood cells for flow cytometric analysis (Yu et al., Whole Blood Leukocytes Isolation with Microfabricated Filter for Cell Analysis. Manuscript submitted to Cytometry).

Materials and Methods

Blood Samples

Blood samples were obtained through BD Blood Donor Program from healthy donors. All samples were anticoagulated with K₃EDTA (Vacutainer; Becton Dickinson). Samples were processed no later than 4 h after venesection, unless indicated differently.

Filtration, Lyse/No Wash, and Lyse/Wash Preparation

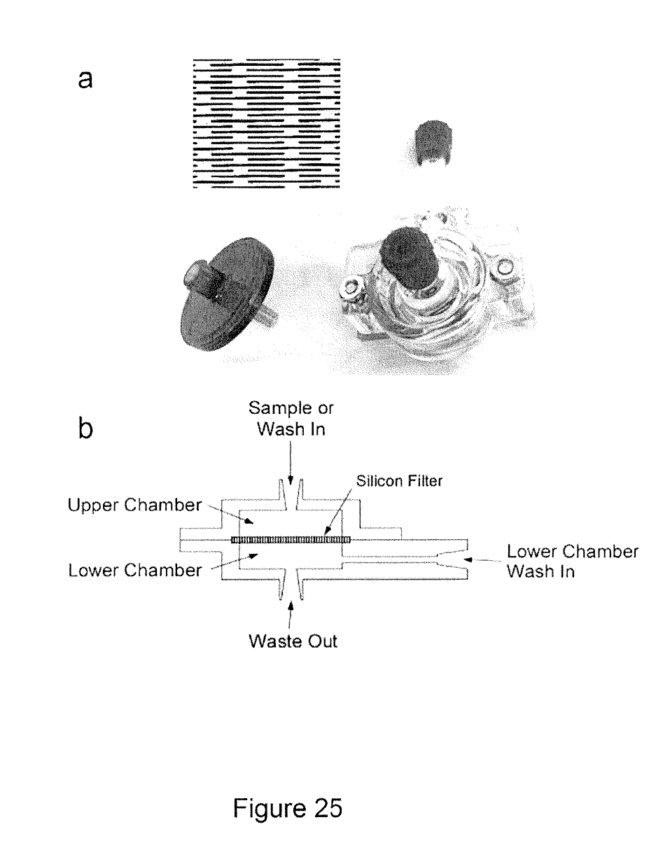

The filter chips and cartridges were manufactured by AVIVA Biosciences (San Diego, Calif.). The microfabricated filters were made from silicon wafer with channels microetched on the chip. The filter cartridge has valves connected to sample reservoir, wash reservoir, and a syringe pump that controls fluid in and out of the cartridge as shown in FIG. 25. Forty devices in two batches (30 in the first batch and 10 in the second batch) were evaluated on performance of leukocyte isolation from healthy donor whole blood. Mainly recovery of leukocyte and subpopulations after filtration, robustness of the filtration process, and cell sustainability after filtration were carefully assessed. Cartridge is recommended for single use; however, it was discovered to be reusable in continuous runs with washing in between. (Reuse was limited to the same donor blood to avoid contamination.)

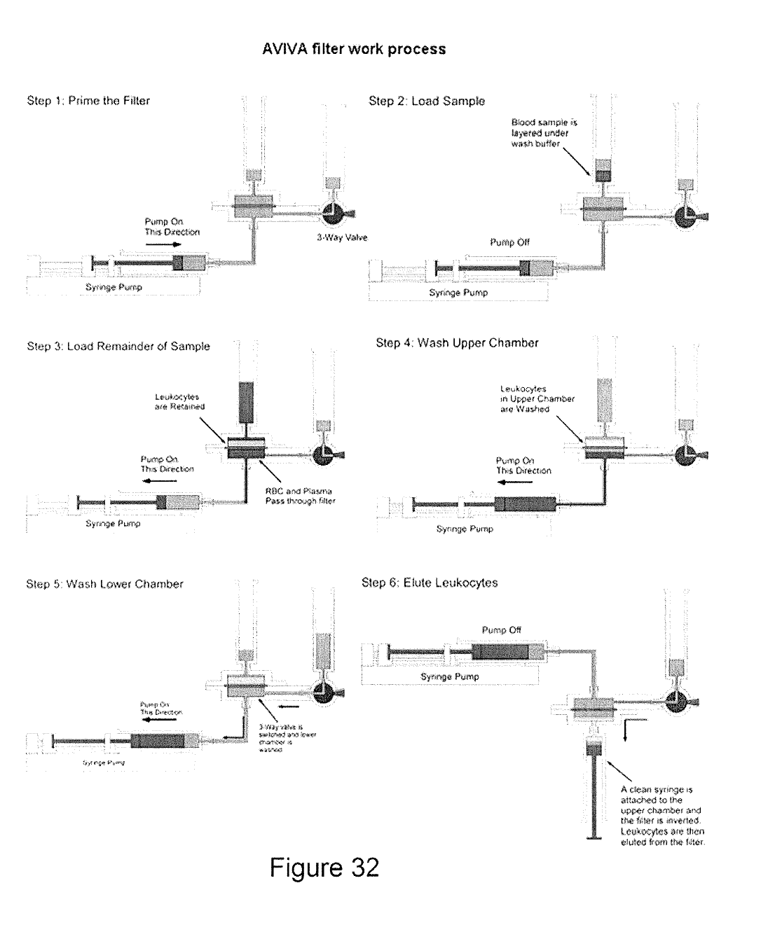

The cartridge was first primed with a proprietary wash buffer, AVIWash-P and then diluted whole blood (10 μl or 50 μl labeled with CD45-PerCP or Multitest™ reagent diluted to 250 μl) was introduced into the upper filter chamber. Buffer or sample solutions were pulled through the filter chip by a syringe pump attached to the lower exit chamber of the device at a speed of either 0.33 or 0.18 ml/min. This was followed by two washing steps: rinsing top of the filter and washing bottom of the filter. Finally, 2 ml of elution buffer was added to the filter cartridge and a 3-ml syringe was used to collect leukocytes that were retained on top of the filter membrane (FIG. 32). The collected leukocytes were transferred to a BD Trucount™ Absolute Counting Tube (cat. 340334) for flow cytometer analysis.

Each blood sample was also tested on an ABX Micros 60 Hematology Analyzer (Horiba ABX) to obtain total leukocyte counts (WBC), erythrocyte counts (RBC), and percent of lymphocytes, monocytes, and granulocytes. ABX counts were used as reference numbers in evaluating recovery of total leukocyte and its three subpopulations from the filtration device.

Fifty μl of each blood sample was also processed following Lyse No Wash Procedure [cell stained with CD45-PerCP (BD Biosciences, San Jose, Calif., cat. 340665) or BD Multitest CD3 FITC/CD16+56 PE/CD45 PerCP/CD19 APC reagent (BD Biosciences, cat. 340500, CD3 Clone SK7, CD16 Clone B73.1, CD56 Clone NCAM 16.2, CD45 Clone 2D1, and CD19 Clone SJ25C1)] and Lyse Wash Procedure following protocols published on BD Biosciences website (http://www.bdbiosciences.com/support/resources/flowcytometry/index.jsp#protocols) with 1×FACS Lysing (BD Biosciences, cat. 349202) solution. Lyse No Wash sample was stained and lysed in Trucount Absolute Counting Tube and Lyse Wash sample was transferred to the Counting Tube after washing.

Cell Viability and Apoptosis Tests

Leucocytes viability after filtration was tested with BD™ Cell Viability Kit (BD Biosciences, cat. 349480). Apoptosis test (Annexin V FITC, BD Biosciences, cat. 556547) was also performed on leukocytes recovered from filtration to test sustainability of the cells.

Flow Cytometer Analysis

Samples were analyzed on Becton Dickinson FACSCalibur™ flow cytometer equipped with BD FACSComp™ and BD CellQuest™ Pro software. The cytometer was calibrated with BD Calibrite™ Calibrite 3 (cat. 340486) and APC (cat. 340487) beads daily by running FACSComp program where cytometer configuration and compensation (Table 1) was set automatically for Lyse No Wash sample and Lyse Wash sample separately. Lyse Wash configuration was applied to filtered sample.

TABLE 1
Cytometer configuration and compensation

| | | Detector | Detector | |
|---|---|---|---|---|
| Laser | Channel | Voltage | Amplification | Mode |
| Blue Laser | FSC | E00 | 2.00 | Linear |
| 488 nm | SSC | 346 | 1.00 | Linear |
| | FL1 (FITC) | 649 | 1.00 | Log |
| | FL2 (PE) | 734 | 1.00 | Log |
| | FL3 (PerCP) | 610 | 1.00 | Log |
| Red Laser | FL4 (APC) | 591 | | Log |
| 635 nm | | | | |

FL1-2.1% FL2, FL2-25.4% FL1, FL2-0.0% FL3, FL3-19.2% FL2, FL3-0.8% FL4, FL4-50.4% FL3

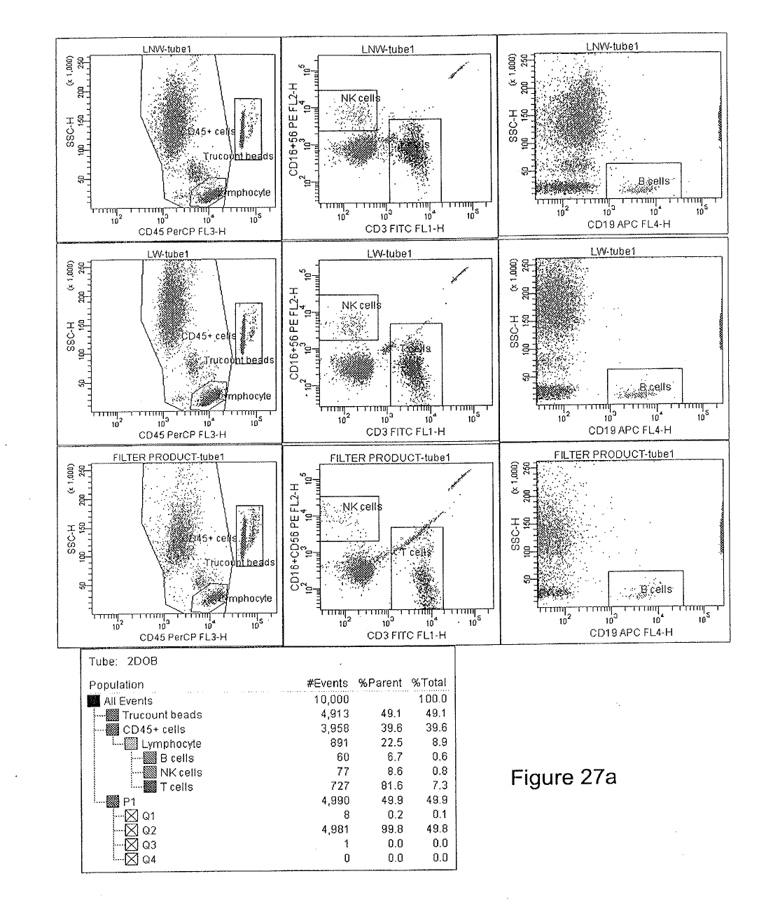

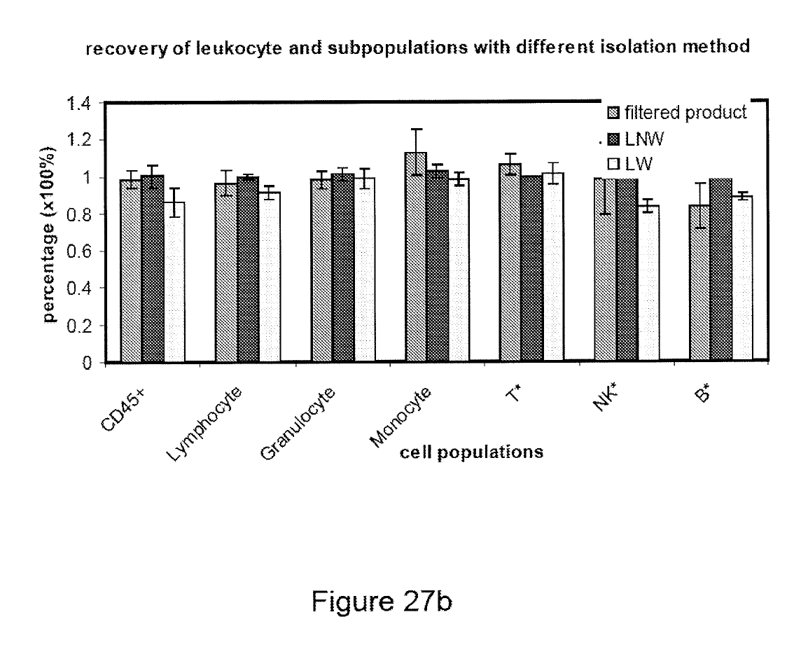

Four fluorescence channels of the cytometer are specified as FL1 FITC, FL2 PE, FL3 PerCP, and FL4 APC. Threshold was set on FL3 (PerCP). Ten thousand total events were acquired for each test unless stated differently. Counting beads were gated on their intense fluorescence signal in FL3 and leukocytes population was gated on CD45+ events in FL3 as well. Lymphocytes, monocytes, and granulocytes were “daughter populations” of leukocytes and were gated based on side scattering and fluorescence. T, B, and NK cells are “daughter populations” of lymphocytes and were further gated based on specific antibody-fluorescent conjugate labeling. In Multitest reagent stained sample, T cells were defined as CD3+ lymphocyte, NK cells were defined as CD16+CD56+ lymphocyte, and B cells were CD19+CD3− lymphocytes (FIG. 27a). All data were analyzed in BD FACSDiva™ software. Absolute cell number was obtained by comparing cell events to Trucount beads event following: Cells per μl=number of cell events×number of beads per tube/number of beads events×sample volume (μl).

Results

Leukocyte Recovery after Filtration and Comparison to Whole Blood Lysis Method

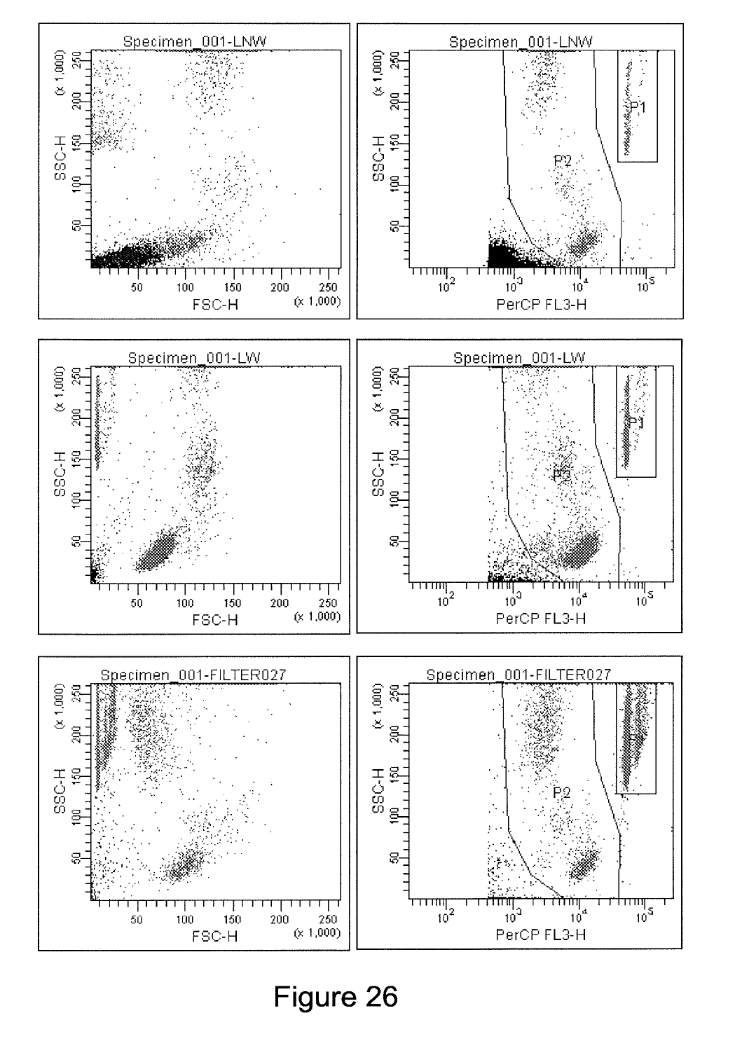

Isolation of leukocytes from whole blood with the microfabricated filter effectively removes red blood cells, which cleans up samples for flow cytometer analysis. FIG. 26 shows dot plots for FSC versus SSC and FL3 versus SSC for the same blood sample prepared following Lyse No Wash procedure, Lyse Wash procedure, and the filtration procedure. The Lyse No Wash sample is substantially contaminated with red cell debris, as can be seen in the dot plot where they represent 91% of the total events acquired. In the Lyse Wash sample, red cell debris are removed through centrifugation and only 13% of the events shown in the dot plot are from debris. Leucocytes recovered from the filtration process contain the smallest percentage of background particles, 4% of the total events; showing that red blood cells are effectively separated from leukocytes.

None or minimum leukocyte cell loss resulted from the filtration process. Leucocyte counts in each sample were calculated with reference to the BD TruCount internal standard counting beads and the overall recovery was based on the ratio of this result to the complete blood count obtained from ABX hematology analyzer. FIG. 27 shows the comparison of recovery results for the total leukocytes, three major leukocyte populations and three lymphocyte subpopulations (T, B, and NK cells). A total of 10 filter cartridges were tested on leukocyte recovery with 10 different donors' blood with each sample run in triplicate on the filter. At its optimum working condition (which is discussed in Table 2), the filter gives on average 98.6%±4.4% recovery of total leukocyte compared to 100.2%±6.0% from LNW and 86.2%±7.8% from LW. The recovery of cells after filtration did not have bias among lymphocyte, monocyte, and granulocyte as compared to blood lysis method. During the evaluation of second batch of filters, fresh blood samples were stained with Multitest reagent to investigate the recovery of subpopulations of lymphocyte, T, B, and NK cells. With five samples, five filters and triplicate of each sample running through each filter, 106%±5.6% recovery of T cells, 98.5%±19% recovery of NK cells, and 83.5%±12% recovery of B cells were observed. Larger deviation of NK cell and B cell recovery could be due to the small percentage of these cells in the blood and limited number of samples.

TABLE 2
Comparison of leukocyte recovery after filtration
at various operation conditions

Flow rate

| Cell load | 0.18 ml/min | 0.33 ml/min |
|---|---|---|
| 10 ul | 0.98 ± 0.04 | 0.92 ± 0.07 |
| (51.1 ± 7.5) × 1000 cells | | |
| 50 ul | 0.75 ± 0.18 | 0.35 ± 0.15 |
| (350 ± 14.1) × 1000 cells | | |

Cell Viability and Sustainability after Filtration

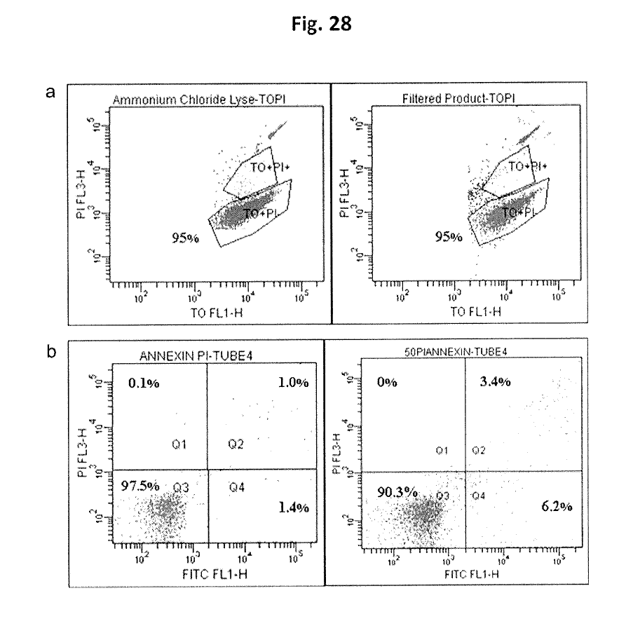

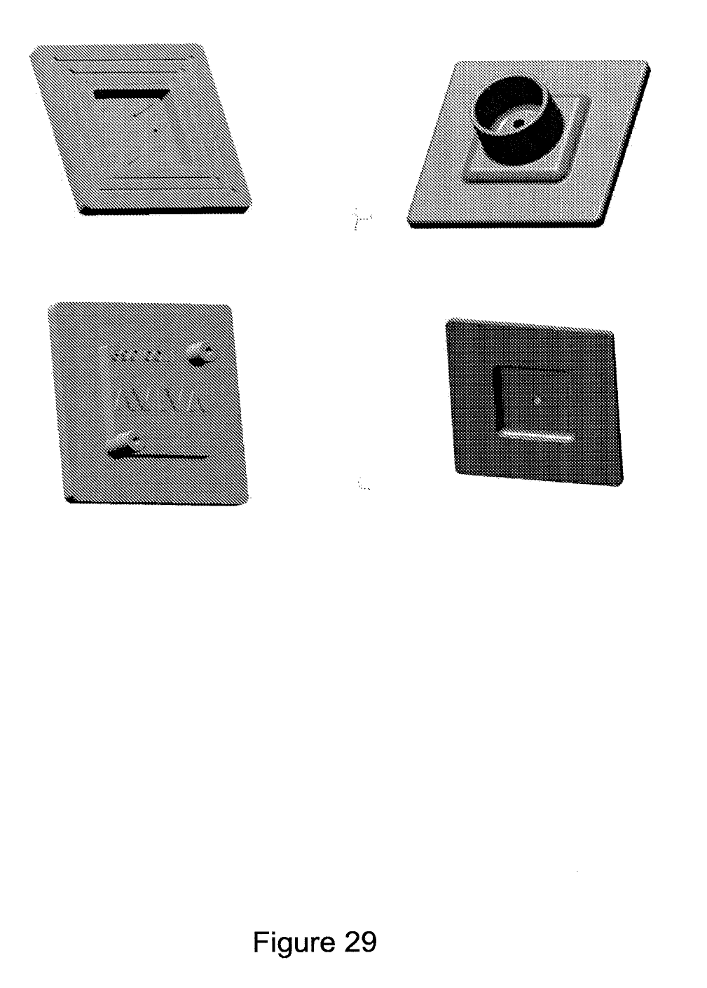

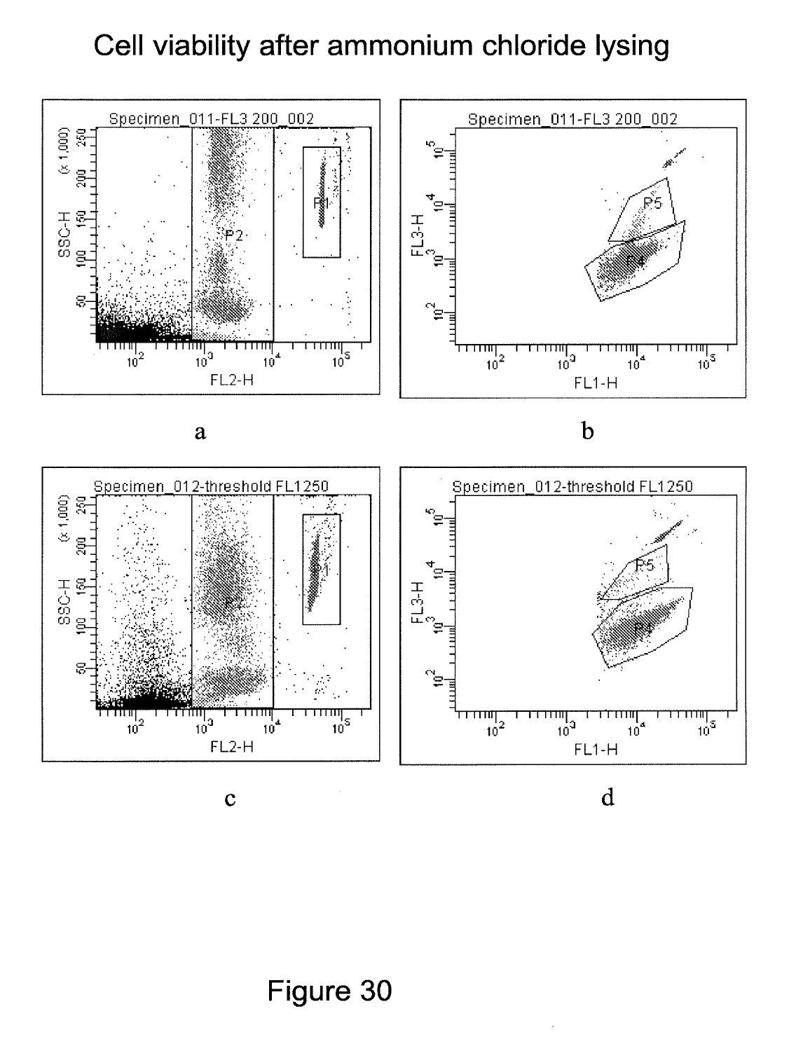

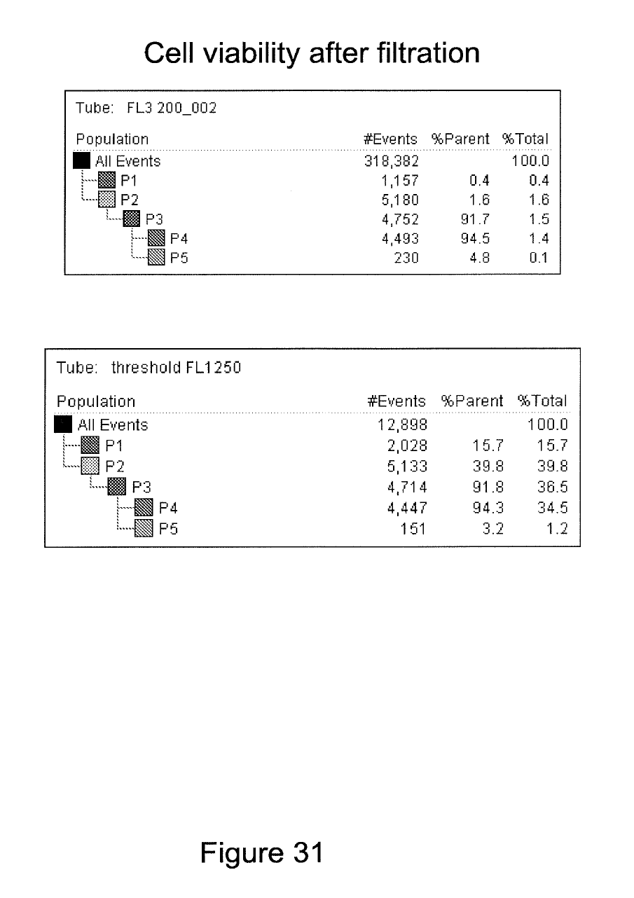

The viability of leukocytes recovered from the filter was tested and compared to that of leukocytes with whole blood lysed with ammonium chloride. FACS lysing solution was not used due to the fact that it contains formaldehyde and therefore leukocytes are fixed during erythrocytes lysis. In both cases, 95% of leukocytes remain alive after erythrocytes are removed and no leukocytes are dead (FIG. 28a). To further test the cells' tolerance of filtration, cells were stained with FITC Annexin V in conjunction with propidium iodide (PI). Annexin V positivity precedes the loss of plasma membrane, which indicates early stage in apoptosis that will lead to cell death (PI positive). Results (FIG. 28b) show that when blood is filtered within an hour of draw, 95% of the cells recovered from filtration show no signs apoptosis; when filtration is performed on blood 8 h after draw, still 90% of the recovered cells remain healthy.

Optimization of Operation Condition

The sample filtration procedure was further fine tuned in order to achieve the best recovery rate. All blood cells were pulled through the filter with a syringe pump set at “pulling” mode, and two different pump rates were tested. As shown in Table 1, at higher flow rate (0.33 ml/min) leukocytes recovery was lower than at lower flow rate (0.18 ml/min) and the effect was more obvious when larger number of cells were loaded on the filter. The pulling force at the higher flow rate might have generated sufficient pressure on the leukocytes to induce physical deformation and passage through the filter's slot. Even when the pump was set at lower flow rate (0.18 ml/min), 50 μl of whole blood with an average of 350,000 leukocytes, which is the typical volume required in BD flow cytometer assays, was pulled through the filter, recovery of leukocytes was not as good as when 10 μl of whole blood with average 50,000 cells was applied. This suggests that, in the configuration tested, the filters may have a finite retention capacity which, when exceeded, leads to cell loss. Results shown in Table 1 were averaged with testing results from at least five filter cartridges for each condition. Further studies will be conducted to determine the optimal relationship between filter size, flow rate, and overall recovery.

Leukocytes isolation methods that depend on erythrocyte lysis are fast and convenient, but may limit analysis options if live cells are needed as FACS lysing solution fixes cells, and ammonium chloride lysis may cause sample degradation if incubation times are not carefully controlled. It is desirable therefore to have an alternative sample preparation method for flow cytometric applications. The microfabricated filter evaluated here is capable of performing fast, simple whole blood separations with high leukocytes recovery without introducing bias among the leukocyte subpopulations. The filter removes erythrocytes, platelets, plasma proteins, and unbound staining reagent. This gentle filtration process produces very clean stained leukocytes for cytometric analysis without any apparent damage to leukocytes. The current filter cartridge is capable of processing the number of cells that are typically required in a flow assay. Its application in flow cytometry sample preparation will help in method standardization, saving labor and material, and minimizing hands-on operation.

Isolation of leukocytes from other components in whole blood is a very important step in flow cytometry cell analysis. Routinely used methods, FICOLL HYPAQUE density gradient centrifugation and red cell lysis, have shown their limitations in applications. We report here the evaluation results of a microfabricated filtration device in blood separation, which potentially provides a new way to prepare stained clean live leukocytes for flow cytometric analysis. The microfabricated filter evaluated here is capable of performing fast, simple whole blood separations with high leukocytes recovery without introducing bias among the leukocyte subpopulations. The filter removes erythrocytes, platelets, plasma proteins, and unbound staining reagent. The results reported here would benefit flow cytometry users with a sample preparation method that allows flow standardization and straightforward operation. For more information, see Yu, Warner, Warner, Recktenwald, Yamanishi, Guia, and Ghetti. Whole blood leukocytes isolation with microfabricated filter for cell analysis. Cytometry A, 79A(12):1009-1015, 2011.

### Example 11

### Method of Separating Nucleated Cells from a Blood Sample Using a Filtration Chamber with Anti-Parallel Flow

An exemplary embodiment of a filtration chamber is depicted in FIG. 33, which has an antechamber and a post-filtration subchamber formed on both sides of a filter by two separate housing parts.

The depth of the antechamber is 400 μm. An embodiment of an antechamber having a depth of about 200 μm or less is also contemplated. In some embodiments the two housing parts may be bound by laser. In some embodiments liquid glue may be used to bond the two housing parts. The top housing part is 34.0 mm×7.9 mm, squared on the inflow side (small port) and rounded on the outflow side (with the large collection well). The outflow receiving well holds 300 μL, has a filtration area of 150×150 mm² and the antechamber holds approximately 65±6 μL of fluid (depending on glue thickness). In embodiments wherein the depth of the antechamber is 200 μm the volume may be ˜30 μL. The inflow port has a 2.4 mm target that funnels down to 1.1 mm port (to engage and seal 19 guage tube or pipette tip or robotic injector tip).

The depth of the post-filtration subchamber is non-uniform, starting at 500 μm on the right for inflow, and ending at 700 μm on the left for the outflow (to correct partially for the increasing concentration of effusate containing the waste cells). The perimeter of the bottom housing part contains a tall well which is meant to prevent contamination of the instrument when in use in the case of accidental overflow of blood on the device or accidental dispensing of the blood outside the inflow port. The largest dimensions at the overflow well are 37.7 mm×11.6 mm. The ports are sized to engage and seal pipes that are 1.1 mm in diameter (19 guage tube) and are spaced about 29.1 mm apart (about 29.0 mm after shrinkage). The post-filtration subchamber is about 400 μm wider than the antechamber to retain any residual glue between the housing parts. The top housing part engages the bottom housing part not only on the horizontal contact surfaces but also for about >1 mm around the perimeter where the quasi-vertical side-walls meet, with a little extra clearance at the corners.

Method of Separating Nucleated Cells from a Blood Sample

Since blood cells are about 10 μm in diameter and make up about 45% of whole blood, the 400 μm depth should allow the cells to pile 25-30-cells deep (not counting platelets). In testing, most of the changes due to filtration happened within the first 115 seconds of filtration. For future testing two filtering modes are used:

1. injecting 50 μL of blood then passing at least 5 volumes of clean media (250-300 μL) over the cells to wash away plasma, platelets, and red blood cells, then recovering in 150 μL of media;

2. pre-filling the chamber with clean media, then slowly and continuously advancing 100 μL of blood through the filter and chasing it with a further volume of clean media while repeatedly applying small pulses of positive pressure from under the filter to keep the retained cells advancing toward the outflow receiving chamber.

In the second mode of filtering, pulse width, pulse height, pulse profile, and duty time will be optimized to recover the leukocytes and rare cells without damage while maximizing removal of red cells, plasma, and platelets.

TABLE 3
Exemplary fluid flow rate for separating and labeling cells

| | | | | | | | | | | | Pump 4 | | Pump 5 | |
|---|---|---|---|---|---|---|---|---|---|---|---|---|---|---|
| | | Pump 1 | | | Pump 2 | | | Pump 3 | | | Recovery | | Filtration | |
| | | Buffer | | | Waste | | | Feed | | | (atmosphere) | | (tandem) | |
| Step | Description | μL | μL/min | sec | μL | μL/min | sec | μL | μL/min | sec | μL | μL/min | μL | μL/min |
| 1 | Filter loading | 150 | 900 | 10 | 167 | 1000 | 10 | 20 | 120 | 10 | 3 | 20 | 17 | 100 |
| | with blood | | | | | | | | | | | | | |
| 2 | Rinse blood | 2250 | 900 | 150 | 2500 | 1000 | 150 | 250 | 100 | 150 | 0 | 0 | 250 | 100 |
| | with buffer | | | | | | | | | | | | | |
| 3 | Add biomarker | 0 | 0 | 0 | 50 | 100 | 30 | −150 | 250 | −36 | −200 | 150 | 50 | 100 |
| | (Optional) | | | | | | | | | | | | | |
| 4 | Loading time | 0 | 0 | 0 | 0 | 0 | 0 | 0 | 0 | 300 | 0 | 0 | 0 | 0 |
| | (Conditional) | | | | | | | | | | | | | |
| 5 | Rinse biomarker | 2250 | 900 | 150 | 2500 | 1000 | 150 | 250 | 100 | 150 | 0 | 0 | 250 | 100 |
| | (Conditional) | | | | | | | | | | | | | |
| 6 | Lift cells | 5 | 5000 | 0 | 0 | 5000 | 0 | 0 | 1000 | 0 | 5 | 1000 | −5 | 0 |
| 7 | Recovery | 35 | 900 | 2 | 35 | 900 | 2 | 195 | 5000 | 2 | 195 | 5000 | 0 | 0 |

Note that controlled elements are bold, whereas derived (calculated) elements are not bold.
NB: Pump 5 = Pump 2 − Pump 1
Pump 4 = Pump 5 − Pump 3

### Example 12

### Automated System for Separating and Analyzing Cells from a Blood Sample

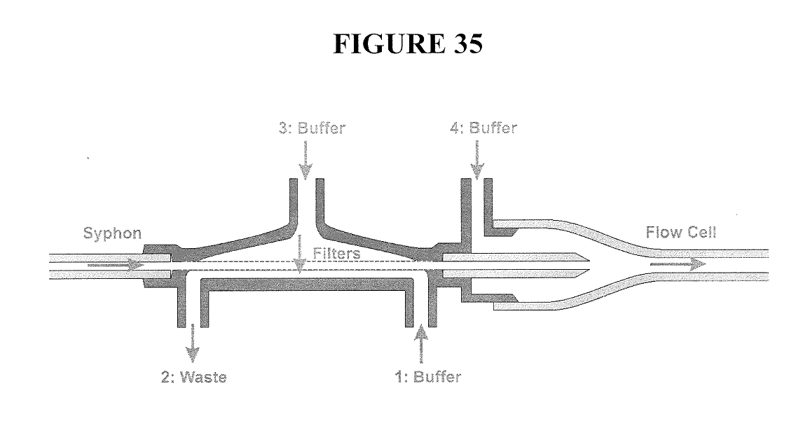

An exemplary embodiment of an automated system is depicted in FIG. 35, which has a filtration chamber directly connected to a flow cytometer.

The syphon picks up the sample cells, preferably a 10× to 100× dilution of whole-blood, or any other mixed cell sample, using environmental pressure as the passive pump.

Pumps 1, 2, and 3 are metered pumps with programmable flow rates that produce the filtration rates. Pump 4 is that which normally produces the concentrated flow (focused flow) of a normal flow cytometer. The flow cell is pumped by vacuum pressure at its distal end.

There are two filters in the filtration chamber, the first is a pre-filter (above the cell flow chamber) which can be any filter and preferably a commercially available SS filter that is coated with our non-stick surface and which only serves to provide directional flow of solution across the filtering chamber as the sample flows through. The second filter is a slotted filter as provided in the present invention, also coated to be non-stick to cells. Plasma, red blood cells, platelets, and unbound markers are removed through the slot filters by the waste pump.

### Example 13

### High-Rinse Capacity Filtration Chamber

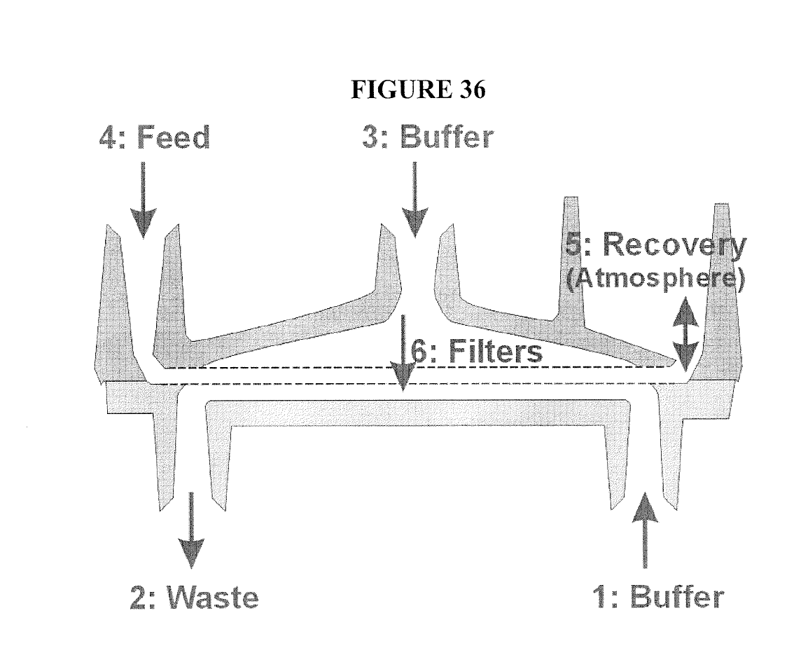

An exemplary embodiment of a high-rinse capacity filtration chamber is depicted in FIG. 36. The high-rinse capacity filtration chamber has two clean buffer entry points (1 and 3) to not only wash away the erythrocytes as they pass through the bottom filter, but to also add clean buffer from above to push more cells through the filter and enable a higher flow rate from the feed pump into the recovery chamber. In this embodiment a pulsatile flow would be preferred where pumps 1 and 2 will alternate between same speed and higher waste outflow, in a coordinated manner with pump 3 alternating between different rates of pump 2−pump 1 and 0. When pump 3 is at 0 flow rate, pumps 1 and 2 will flow at the same rate. This will allow the feed pump 4 to intermittently and gradually push the retained cells across the filter and into the recovery chamber as they become de-bulked of plasma, thrombocytes, erythrocytes, unbound markers, soluble antigens, etc. There are two filters in this arrangement, the bottom filter is a slotted filter and the top filter may be any common filter that will retain its flatness in the low flow conditions and that may be coated with a non-stick surface as needed. The top filter could, for example, be a stainless steel sheet or a polyimide sheet with holes of any shape which are approximately 0.05 to 2 microns in diameter. The top filter may be supported by structures on the buffer distribution chamber above it to maintain its flatness during filtration.

The recovery pump is imaginary (atmospheric pressure) and its flow rate may be calculated by pump 4−pump 2+pump 1+pump 3. The filtering pump (of the slotted filter on the bottom) is imaginary (controlled by other pumps working in tandem) and its flow rate may be calculated by pump 2−pump 1.

### Example 14

### Two Filtration Chambers in Tandem

An exemplary embodiment of two filtration chambers in tandem is depicted in FIG. 37. The two filtration chambers are in fluid connection between the two filters overlapping each other, i.e., the antechamber.

### Example 15

### Filtration Chamber with Multiple Output Ports

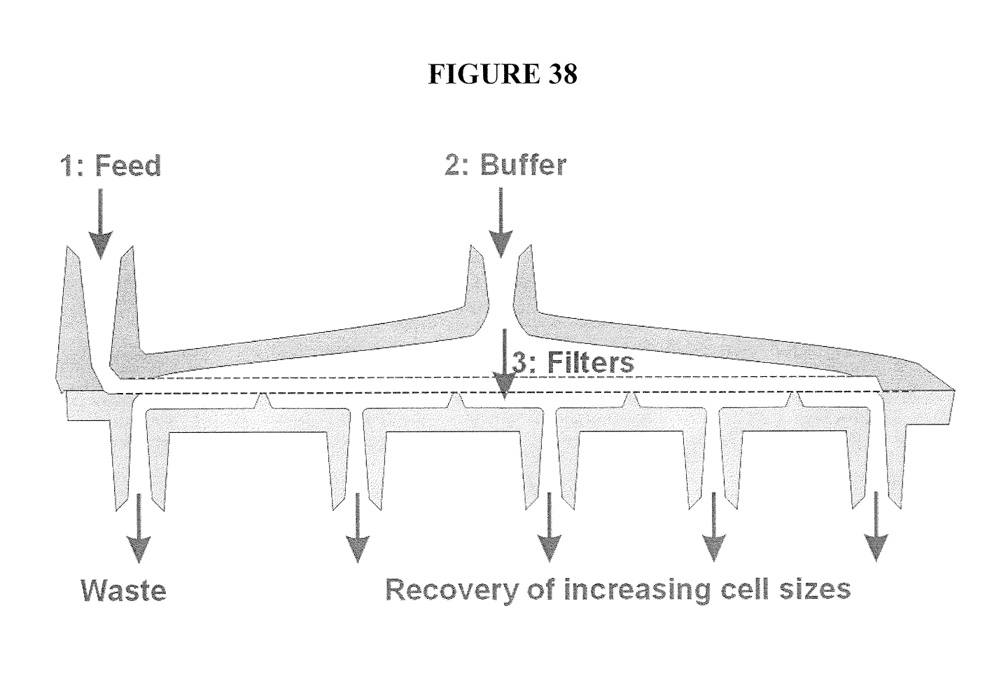

An exemplary embodiment of a filtration chamber with multiple output ports is depicted in FIG. 38. Two or more filters in tandem with slot widths of increasing size for each filter may be enclosed in a filtration chamber. It may also be possible to use a single, longer filter with multiple output ports on the bottom to remove sequentially larger cells along the path through the top chamber.

### Example 16

### Manufacturing of Filters from Whole-Wafer Filter Membranes

A Silicon wafer was bonded to a glass wafer that was to act as a sacrificial carrier, then was thinned, masked, and etched to produce a continuous filter on the entire surface of the wafer, using the following steps.

The bonding compound was spin-coated to a uniform thickness onto a sacrificial glass wafer and the silicon wafer was pressed onto the sacrificial wafer to eliminate bubbles during curing and the glue was baked to cure.

The attached silicon wafer was them thinned by CMP until its thickness across its entire surface was 40 to 60 μm, and specifically 55 μm to 60 μm in thickness.

A dielectric layer such as silicon dioxide was then depositioned onto the silicon wafer to function as a hard mask.

A polymer mask layer (soft mask) was then layered on top of the hard mask by spin-coating method, and solidified on a hot-plate.

The soft mask was then pattered across its entire surface using a projection mask such that the entire surface was cured by ultraviolet light except for the repeating rectangular areas that would become the slots.

The uncured soft mask material and the exposed hard mask under it were etched away.

The wafer was then deep-etched using deep reactive ion etching, DRIE, process according to the Bosch method. This process removed the soft mask and etched the patterned slots through the wafer and was continued to remove some of the underlying wafer bonding compound between the two wafers. The mask sizing and DRIE process were configured such that the resulting slots were 2.8 μm wide by 55-60 μm deep by 50 μm long and repeating over the entire surface of the wafer every 9 μm along its short axis and every 70 μm along its long axis. The perimeter of the wafer had an un-etched ring area of 5 mm from the edge which resulted in a stronger perimeter edge that could be used for handling later.

The wafer was then placed into a plasma-enhanced vapor deposition chamber and TiN was depositioned onto its entire surface.

The bonding compound between the sacrificial wafer and the filter wafer was dissolved using oxygen-free 1-dodecene until the filter wafer was released and floated off of the sacrificial wafer (which could then be re-used for additional wafers).

The liberated filter wafer was rinsed well in methanol then placed into a vacuum oven to dry.

The wafer was then bonded to a plastic handling ring as well as to one side of the injection-molded plastic filter body housings that had also been deposition coated with TiN.

After bonding, the housings were snapped apart retaining the bonded segments of filter, and assembled to the second half of the molded filter housings, also deposition-coated with TiN, to produce ready-to-use filters.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

We claim:

1. A filtration chamber comprising a microfabricated filter enclosed in a housing, wherein the filtration chamber comprises an antechamber and a post-filtration subchamber, and the fluid flow path in the antechamber is substantially opposite to the fluid flow path in the post-filtration subchamber.

2. The filtration chamber of claim 1, wherein each of the antechamber and the post-filtration subchamber has an inflow port and/or an outflow port.

3. The filtration chamber of claim 2, wherein the antechamber comprises at least two inflow ports.

4. The filtration chamber of claim 3, wherein the antechamber comprises a suprafilter thereby creating a suprachamber.

5. The filtration chamber of claim 4, wherein the suprafilter, between the antechamber and the suprachamber, is sufficiently rigid to maintain its flatness under slow flow conditions.

6. The filtration chamber according to claim 4 or 5, wherein the suprafilter comprises holes or slots with openings smaller than about 5 microns.

7. The filtration chamber according to any one of claims 2-6, wherein the inflow port and outflow port may be used interchangeably.

8. The filtration chamber according to any one of claims 1-7, wherein the microfabricated filter comprises one or more tapered slots.

9. The filtration chamber of claim 8, wherein the microfabricated filter comprises from about 100 to 5,000,000 tapered slots.

10. The filtration chamber according to any one of claims 1-9, wherein the thickness of the microfabricated filter is from about 20 to about 200 microns.

11. The filtration chamber of claim 10, wherein the thickness of the microfabricated filter is from about 40 to about 70 microns.

12. The filtration chamber according to any one of claims 8-11, wherein the tapered slots are from approximately 20 microns to 200 microns in length and from about 2 microns to about 16 microns in width, and the tapering of said slots is from about 0 degree to about 10 degrees, and wherein the variation in slot size of said tapered slot is less than about 20%.

13. The filtration chamber according to any one of claims 8-11, wherein the size of the tapered slots varies by more than 20%.

14. The filtration chamber of claim 13, wherein the size of the tapered slots varies by more than 50%.

15. The filtration chamber of claim 14, wherein the size of the tapered slots varies by more than 100%.

16. The filtration chamber according to any one of claims 13-15, wherein the size of the tapered slots varies along the fluid flow path in the antechamber.

17. The filtration chamber according to any one of claims 2-16, wherein the post-filtration subchamber comprises at least two outflow ports.

18. The filtration chamber of claim 17, wherein the at least two outflow ports are arranged along the fluid flow path in the antechamber.

19. The filtration chamber according to any one of claims 1-18, comprising two or more electrodes.

20. The filtration chamber of claim 19, wherein the electrodes are placed on opposite sides of the microfabricated filter.

21. The filtration chamber according to claim 19 or 20, wherein the electrodes are placed on the housing of the filtration chamber.

22. The filtration chamber according to any one of claims 19-21, wherein the electrodes are placed in the antechamber and/or the post-filtration subchamber.

23. The filtration chamber according to any one of claims 19-21, wherein the electrodes are incorporated or placed into one or more of the ports or connections that interact with the antechamber and/or the post-filtration subchamber.

24. The filtration chamber according to any one of claims 1-23, wherein the filtration chamber comprises at least one acoustic element.

25. The filtration chamber according to any one of claims 1-24, wherein the outflow port of the antechamber is connected to a collection chamber or collection well.

26. The filtration chamber according to any one of claims 1-25, wherein the housing comprises a top part and a bottom part, and the top part and the bottom part engage or bond together to form the filtration chamber.

27. The filtration chamber according to any one of claims 1-26, wherein the filtration chamber has a length of about 1 mm to about 10 cm, a width of about 1 mm to about 3 cm, and a depth of about 0.02 mm to about 20 mm.

28. The filtration chamber of claim 27, wherein the filtration chamber has a length of about 10 mm to about 50 mm, a width of about 5 mm to about 20 mm, and a depth of about 0.05 mm to about 2.5 mm.

29. The filtration chamber of claim 28, wherein the filtration chamber has a length of about 30 mm, a width of about 6 mm, and a depth of about 1 mm.

30. The filtration chamber according to any one of claims 1-29, wherein the housing has a length of about 38 mm, a width of about 12 mm, and a depth of about 20 mm as outer dimensions.

31. The filtration chamber according to any one of claims 27-30, wherein its antechamber has a length of about 1 mm to about 10 cm, a width of about 1 mm to about 3 cm, and a depth of about 0.01 mm to about 10 mm.

32. The filtration chamber of claim 31, wherein its antechamber has a length of about 10 mm to about 50 mm, a width of about 5 mm to about 20 mm, and a depth of about 0.01 mm to about 1 mm.

33. The filtration chamber of claim 32, wherein its antechamber has a length of about 30 mm, a width of about 6 mm, and a depth of about 0.1-0.4 mm.

34. The filtration chamber according to any one of claims 31-33, wherein the volume of the antechamber is about 0.01 μL to about 5 mL.

35. The filtration chamber of claim 34, wherein the volume of the antechamber is about 1 μL to about 100 μL.

36. The filtration chamber of claim 35, wherein the volume of the antechamber is about 40 to 80 μL.

37. The filtration chamber according to any one of claims 27-36, wherein the post-filtration subchamber has a length of about 1 mm to about 10 cm, a width of about 1 mm to about 3 cm, and a depth of about 0.01 mm to about 1 cm.

38. The filtration chamber of claim 37, wherein the post-filtration subchamber has a length of about 10 mm to about 50 mm, a width of about 5 mm to about 20 mm, and a depth of about 0.2 mm to about 1.5 mm.

39. The filtration chamber of claim 38, wherein the post-filtration subchamber has a length of about 30 mm, a width of about 6.4 mm, and a depth of about 0.6-1 mm.

40. A filtration chamber comprising a microfabricated filter enclosed in a housing, wherein the surface of said filter and/or the inner surface of said housing are modified by vapor deposition, sublimation, vapor-phase surface reaction, or particle sputtering to produce a uniform coating.

41. The filtration chamber of claim 40, wherein the filtration chamber comprises an antechamber and a post-filtration subchamber.

42. The filtration chamber of claim 41, wherein the antechamber comprises a suprafilter thereby creating a suprachamber.

43. The filtration chamber of claim 42, wherein the surface of the suprafilter is modified by vapor deposition, sublimation, vapor-phase surface reaction, or particle sputtering to produce a uniform coating.

44. The filtration chamber according to any one of claims 40-43, wherein the modification is by physical vapor deposition.

45. The filtration chamber according to any one of claims 40-43, wherein the modification is by plasma-enhanced chemical vapor deposition.

46. The filtration chamber according to any one of claims 40-43, wherein the vapor deposition is of a metal nitride or a metal halide.

47. The filtration chamber of claim 46, wherein the metal nitride is titanium nitride, silicon nitride, zinc nitride, indium nitride, and/or boron nitride.

48. The filtration chamber according to any one of claims 40-43, wherein the modification is by chemical vapor deposition.

49. The filtration chamber of claim 48, wherein the chemical vapor deposition is by a Parylene or derivative thereof.

50. The filtration chamber of claim 49, wherein the Parylene or derivative thereof is selected from the group consisting of Parylene, Parylene-N, Parylene-D, Parylene AF-4, Parylene SF, and Parylene HT.

51. The filtration chamber of claim 48, wherein the modification is by polytetrafluoroethylene (PTFE).

52. The filtration chamber of claim 48, wherein the modification is by Teflon-AF.

53. The filtration chamber according to claim 40 or 43, wherein the modification is by a perfluorocarbon.

54. The filtration chamber of claim 53, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane and is in liquid form.

55. The filtration chamber according to any one of claims 40-54, wherein the filter and/or housing comprises silicon, silicon dioxide, glass, metal, carbon, ceramics, plastic, or a polymer.

56. The filtration chamber according to any one of claims 40-54, wherein the filter and/or housing comprises silicon nitride or boron nitride.

57. A filtration chamber comprising a microfabricated filter enclosed in a housing, wherein the surface of said filter and/or the inner surface of said housing are modified by a metal nitride, a metal halide, a Parylene or derivative thereof, a polytetrafluoroethylene (PTFE), a Teflon-AF or a perfluorocarbon.

58. The filtration chamber of claim 57, wherein the filtration chamber comprises an antechamber and a post-filtration subchamber.

59. The filtration chamber of claim 58, wherein the antechamber comprises a suprafilter thereby creating a suprachamber.

60. The filtration chamber of claim 59, wherein the surface of the suprafilter is modified by a metal nitride, a metal halide, a Parylene or derivative thereof, a polytetrafluoroethylene (PTFE), a Teflon-AF or a perfluorocarbon.

61. The filtration chamber according to any one of claims 57-60, wherein the metal nitride is titanium nitride, silicon nitride, zinc nitride, indium nitride, and/or boron nitride.

62. The filtration chamber according to any one of claims 57-60, wherein the Parylene or derivative thereof is selected from the group consisting of Parylene, Parylene-N, Parylene-D, Parylene AF-4, Parylene SF, and Parylene HT.

63. The filtration chamber according to any one of claims 57-60, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane, and the perfluorocarbon covalently binds the surface.

64. The filtration chamber according to any one of claims 57-63, wherein the filter and/or housing comprises silicon, silicon dioxide, glass, metal, carbon, ceramics, plastic, or a polymer.

65. The filtration chamber according to any one of claims 57-63, wherein the filter and/or housing comprises silicon nitride or boron nitride.

66. A filtration chamber according to any one of claims 1-39, wherein the surface of the filter and/or the inner surface of said housing are modified by vapor deposition, sublimation, vapor-phase surface reaction, or particle sputtering to produce a uniform coating.

67. The filtration chamber of claim 66, wherein the vapor deposition is of a metal nitride or a metal halide.

68. The filtration chamber of claim 67, wherein the metal nitride is titanium nitride, silicon nitride, zinc nitride, indium nitride, and/or boron nitride.

69. The filtration chamber of claim 66, wherein the modification is by chemical vapor deposition.

70. The filtration chamber of claim 66, wherein the modification is by a perfluorocarbon.

71. The filtration chamber of claim 70, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane and is in liquid form.

72. A filtration chamber according to any one of claims 1-39, wherein the surface of the filter and/or the inner surface of said housing are modified by a metal nitride, a metal halide, a Parylene, a polytetrafluoroethylene (PTFE), a Teflon-AF or a perfluorocarbon.

73. The filtration chamber of claim 72, wherein the metal nitride is titanium nitride, silicon nitride, zinc nitride, indium nitride, and/or boron nitride.

74. The filtration chamber of claim 72, wherein the perfluorocarbon is 1H,1H,2H,2H-perfluorooctyltriethoxysilane, 1H,1H,2H,2H-perfluorodecyltriethoxysilane, trichloro(1H,1H,2H,2H-perfluorooctyl)silane or trichloro(octadecyl)silane, and the perfluorocarbon covalently binds the surface.

75. A filtration chamber according to any one of claims 1-74, comprising at least two microfabricated filters.

76. The filtration chamber of claim 75, wherein the at least two microfabricated filters are arranged in tandem.

77. A filtration chamber comprising at least two filtration chambers according to any one of claims 1-76 arranged in tandem.

78. The filtration chamber of claim 77, wherein the antechambers of the at least two filtration chambers are in fluid connection.

79. The filtration chamber of claim 78, wherein the at least two filtration chambers share one microfabricated filter and/or suprafilter.

80. The filtration chamber according to claim 77 or 78, wherein the slots of the filters within each filtration chamber are of different widths, and the filtration chambers are arranged in order of increasing slot widths.

81. A cartridge comprising the filtration chamber according to any one of claims 1-80.

82. The cartridge of claim 81, comprising at least two filtration chambers.

83. The cartridge of claim 82, comprising eight filtration chambers.

84. An automated filtration unit for separating a target component in a fluid sample, comprising the filtration chamber according to any one of claims 1-80.

85. The automated filtration unit of claim 84, further comprising a control algorithm for controlling the fluid flow in the filtration chamber.

86. The automated filtration unit according to claim 84 or 85, comprising at least two filtration chambers.

87. The automated filtration unit according to claim 86, wherein the at least two filtration chambers are arranged in tandem, and the filtration chambers comprise filters of increasing slot width.

88. The automated filtration unit according to claim 86 or 87, wherein the filters contain slot widths of increasing size along the fluidic path.

89. The automated filtration unit of claim 88, comprising a suprachamber.

90. The automated filtration unit according to any one of claims 84-89, wherein the post-filtration subchamber comprises multiple partitions each comprising an outflow port.

91. The automated filtration of claim 90, wherein the outflow port from each partition of the post-filtration chamber is aligned with individual wells of a multi-well plate.

92. The automated filtration of claim 91, wherein the wells are spaced about every 1-100 mm.

93. The automated filtration of claim 91, wherein the wells are spaced about every 2.25 mm.

94. The automated filtration of claim 91, wherein the wells are spaced about every 4.5 mm.

95. The automated filtration of claim 91, wherein the wells are spaced about every 9 or 18 mm.

96. The automated filtration unit according to any one of claims 84-95, comprising eight filtration chambers.

97. The automated filtration unit according to any one of claims 84-96, comprising a means for effecting fluid flow in the filtration chamber.

98. The automated filtration unit of claim 97, wherein the means for effecting fluid flow is a fluidic pump.

99. The automated filtration unit according to any one of claims 84-98, comprising a means for collecting the separated target component.

100. An automated system for separating and analyzing a target component in a fluid sample, comprising the automated filtration unit according to any one of claims 84-98 and an analysis apparatus connected to the filtration unit.

101. The automated system of claim 100, wherein the analysis apparatus is a cell sorting device or a flow cytometer.

102. A method for separating a target component in a fluid sample, comprising:
a) dispensing the fluid sample into the filtration chamber according to any one of claims 1-80; and
b) providing a fluid flow of the fluid sample through the filtration chamber, wherein the target component of the fluid sample is retained by or passes through the filter.

103. The method of claim 102, comprising providing a fluid flow of the fluid sample through the antechamber of the filtration chamber and a fluid flow of a solution through the post-filtration subchamber of the filtration chamber, and optionally a fluid flow of a solution through the suprachamber of the filtration chamber.

104. The method according to claim 102 or 103, wherein the fluid sample is separated based on the size, shape, deformability, binding affinity and/or binding specificity of the components.

105. The method according to claim 103 or 104, wherein the fluid sample is dispensed through the inflow port of the antechamber.

106. The method according to any one of claims 103-105, wherein the solution is introduced to the inflow port of the post-filtration subchamber.

107. The method according to any one of claims 103-105, wherein the solution is introduced to the inflow port of the supra-filtration chamber.

108. The method according to any one of claims 102-107, wherein the fluid sample is manipulated by a physical force effected via a structure that is external to the filter and/or a structure that is built-in on the filter.

109. The method of claim 108, wherein the physical force is selected from the group consisting of a dielectrophoretic force, a traveling-wave dielectrophoretic force, a magnetic force, an acoustic force, an electrostatic force, a mechanical force, an optical radiation force and a thermal convection force.

110. The method of claim 109, wherein the dielectrophoretic force or the traveling-wave dielectrophoretic force is effected via an electrical field produced by an electrode.

111. The method of claim 109, wherein the acoustic force is effected via a standing-wave acoustic field or a traveling-wave acoustic field.

112. The method of claim 109, wherein the acoustic force is effected via an acoustic field produced by piezoelectric material.

113. The method of claim 109, wherein the acoustic force is effected via a voice coil or audio speaker.

114. The method of claim 109, wherein the electrostatic force is effected via a direct current (DC) electric field.

115. The method of claim 109, wherein the optical radiation force is effected via laser tweezers.

116. The method according to any one of claims 102-115, wherein the fluid sample is blood, effusion, urine, bone marrow sample, ascitic fluid, pelvic wash fluid, pleural fluid, spinal fluid, lymph, serum, mucus, sputum, saliva, semen, ocular fluid, extract of nasal, throat or genital swab, cell suspension from digested tissue, extract of fecal material, cultured cells of either mixed types and/or mixed sizes, or cells that contain contaminants or unbound reactants that need to be removed.

117. The method of claim 116, wherein the fluid sample is a blood sample and the component being removed is a plasma, a platelet and/or a red blood cell (RBC).

118. The method of claim 116, wherein the fluid sample are cells that contain contaminants or unbound reactants that need to be removed, and the reactant is a labeling reagent for the cells.

119. The method of claim 116, wherein the fluid sample is a blood sample and the target component is a nucleated cell, e.g., a non-hematopoietic cell, a subpopulation of blood cells, a fetal red blood cell, a stem cell, or a cancerous cell.

120. The method of claim 116, wherein the fluid sample is an effusion or a urine sample and the target component is a nucleated cell, e.g., a cancerous cell or a non-hematopoietic cell.

121. A method of separating a target component in a fluid sample using the automated filtration unit according to any one of claims 84-99, comprising:
a) dispensing the fluid sample into the filtration chamber; and
b) providing a fluid flow of the fluid sample through the filtration chamber, wherein the target component of the fluid sample is retained by or flows through the filter.

122. The method of claim 121, wherein the fluid sample is separated based on the size, shape, deformability, binding affinity and/or binding specificity of the components.

123. The method according to claim 121 or 122, wherein the fluid sample in the antechamber flows substantially anti-parallel to the solution in the post-filtration subchamber.

124. The method according to any one of claims 121-123, wherein the filter rate is about 0-5 mL/min.

125. The method of claim 124, wherein the filter rate is about 10-500 μL/min.

126. The method of claim 125, wherein the filter rate is about 80-140 μL/min.

127. The method according to any one of claims 124-126, wherein the feed rate is about 1-10 times the filter rate.

128. The method according to any one of claims 102-127, further comprising:
c) rinsing the retained components of the fluid sample with an additional sample-free rinsing reagent.

129. The method of claim 128, wherein during the rinsing step the feed rate is less than or equal to the filter rate.

130. The method according to claim 128 or 129, wherein a rinsing reagent is introduced to the post-filtration subchamber.

131. The method according to claim 128 or 129, wherein the rinsing reagent is introduced to the antechamber and/or the suprachamber.

132. The method according to any one of claims 102-131, further comprising:
d) providing a labeling reagent to bind to the target component.

133. The method of claim 132, wherein the labeling reagent is an antibody.

134. The method according to claim 132 or 133, wherein the labeling reagent is added to the collection chamber.

135. The method according to claim 132 or 133, wherein the labeling reagent is added to the antechamber and/or the suprachamber.

136. The method according to any one of claims 132-135, wherein during the labeling step the fluid flow in the post-filtration subchamber is stopped.

137. The method according to any one of claims 132-136, further comprising:
e) removing the unbound labeling reagent.

138. The method according to any one of claims 102-137, further comprising:
f) recovering the target component in the collection chamber.

139. The method of claim 138, wherein during the recovering step the feed rate is about 5-20 mL/min.

140. The method according to claim 138 or 139, wherein during the recovering step the outflow rate equals the inflow rate in the post-filtration subchamber.

141. The method according to any one of claims 138-140, wherein during the recovering step the outflow is paused for about 50 ms.

142. The method according to any one of claims 121-141, wherein the fluid sample is a blood sample, which comprises removing at least one type of undesirable component using a specific binding member.

143. The method of claim 142, wherein the at least one undesirable component are white blood cells (WBCs).

144. The method of claim 143, wherein the specific binding member selectively binds to WBCs and is coupled to a solid support.

145. The method of claim 144, wherein the specific binding member is an antibody or an antibody fragment that selectively binds to WBCs.

146. The method of claim 145, wherein the specific binding member is an antibody that selectively binds to CD3, CD11b, CD14, CD17, CD31, CD45, CD50, CD53, CD63, CD69, CD81, CD84, CD102 or CD166.

147. The method of claim 146, wherein the specific binding member is an antibody that selectively binds to CD35 and/or CD50.

148. The method according to any one of claims 142-147, further comprising contacting the blood sample with a secondary specific binding member.

149. The method of claim 148, wherein said secondary specific binding member is an antibody that selectively binds to CD31, CD36, CD41, CD42 (a, b or c), CD51, or CD51/61.

150. A method of enriching and analyzing a target component in a fluid sample using the automated system according to claim 100 or 101, comprising,
a) dispensing the fluid sample into the filtration chamber;
b) providing a fluid flow of the fluid sample through the antechamber of the filtration chamber and a fluid flow of a solution through the post-filtration subchamber of the filtration chamber, wherein the target component of the fluid sample is retained in the antechamber and non-target components flow through the filter into the post-filtration subchamber;
c) labeling the target component; and
d) analyzing the labeled target component using the analysis apparatus.

151. The method of claim 150, comprising providing fluid flow into the suprachamber.

152. The method according to claim 150 or 151, wherein the target component is a cell or cellular organelle.

153. The method of claim 152, wherein the cell is a nucleated cell.

154. The method of claim 152, wherein the cell is a rare cell.
